SEQUENCE LISTING (24 sequences)

SEQ ID NO: 1 - DNA, 85 bp, Saccharomyces cerevisiae
gggagtttggccgagtggtttaaggcgtcagatttaggctctgatatcttcggatgcaagggttcgaatcccttagctctcacca

SEQ ID NO: 2 - DNA, 77 bp, Saccharomyces cerevisiae
gaaactataattcaattggttagaatagtattttgataaggtacaaatataggttcaatccctgttagtttcatcca

SEQ ID NO: 3 - protein, 258 aa, Artificial Sequence
  Editing Domain of a tRNA Synthetase Molecule
TPQEYIGVKIEALEFADDAAKIIDSSSDLDKSKKFYFVAATLRPETMYGQTCCFVSPTIEYGIFDAGDSYFITTERAFKNMSYQKLTPKRGFYKPIVTVPGKAFIGTKIHAPQSVYPELRILPMETVIATKGTGVVTCVPSNSPDDYITTKDLLHKPEYYGIKPEWIDHEIVPIMHTEKYGDLTAKAIVEEKKIQSPKDKNLLAEAKKIAYKEDYYTGTMIYGPYKGEKVEQAKNKVKADMIAAGEAFVYNEPESQDP

SEQ ID NO: 4 - protein, 280 aa, Artificial Sequence
  Editing Domain of a tRNA Synthetase Molecule
MTPQEYIGVKIEALEFADDAAKIIDSSSDLDKSKKFYFVAATLRPETMYGQTCCFVSPTIEYGIFDAGDSYFITTERAFKNMSYQKLTPKRGFYKPIVTVPGKAFIGTKIHAPQSVYPELRILPMETVIATKGTGVVTCVPSNSPDDYITTKDLLHKPEYYGIKPEWIDHEIVPIMHTEKYGDLTAKAIVEEKKIQSPKDKNLLAEAKKIAYKEDYYTGTMIYGPYKGEKVEQAKNKVKADMIAAGEAFVYNEPESQDPQDPNSSSVDKLAAALEHHHHH

SEQ ID NO: 5 - protein, 303 aa, Artificial Sequence
  Editing Domain of a tRNA Synthetase Moleucle
TCTPEYYRWEQKFFTELYKKGLVYKKTSAVNWCPNDQTVLANEQVIDGCCWRCDTKVERKEIPQWFIKITAYADELLNDLDKLDHWPDTVKTMQRNWIGRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYRLRDWGVSRQRYWG

SEQ ID NO: 6 - protein, 308 aa, Artificial Sequence
  Editing Domain of a tRNA Synthetase Molecule
TCKPDYYRWEQWLFTRLFEKGVIYRKNGTVNWDPADQTVLANEQVIDGRGWRSGALIEKREIPMYYFRITDYADELLESLDELPGWPEQVKTMQRNWIGKSRGMEVQFPYDQASIGHEGTLKVFTTRPDTLMGATYVAVAAEHPLATQAAQGNAALQAFIDECKSGSVAEADMATQEKKGMATSLFVEHPLTGEKLPVWVANYVLMHYGDGAVMAVPAHDERDFEFAHKYNLPVKAVVRTSAGDDVGSEWLAAYGEHGQLINSGEFDGLDFQGAFDAIEAALIRKDLGKSRTQFRLRDWGISRQRYWG

SEQ ID NO: 7 - protein, 298 aa, Artificial Sequence
  Editing Domain of a tRNA Synthetase Molecule
TTDPEYYKWTQWIFIQLYNKGLAYVDEVAVNWCPALGTVLSNEEVIDGVSERGGHPVYRKPMKQWVLKITEYADQLLADLDDLDWPESLKDMQRNWIGRSEGAKVSFDVDNTEGKVEVFTTRPDTIYGASFLVLSPEHALVNSITTDEYKEKVKAYQTEASKKSDLERTDLAKDKSGVFTGAYAINPLSGEKVQIWIADYVLSTYGTGAIMAVPAHDDRDYEFAKKFDLLIIEVIEGGNVEEAAYTGEGKHINSGELDGLENEAAITKAIQLLEQKGAGEKKVYKLRDWLFSRQRYWG

SEQ ID NO: 8 - protein, 192 aa, Escherichia coli
GRSEGVEITFNVNDYDNTLTVYTTRPDTFMGCTYLAVAAGHPLAQKAAENNPELAAFIDECRNTKVAEAEMATMEKKGVDTGFKAVHPLTGEEIPVWAANFVLMEYGTGAVMAVPGHDQRDYEFASKYGLNIKPVILAADGSEPDLSQQALTEKGVLFNSGEFNGLDHEAAFNAIADKLTAMGVGERKVNYR

SEQ ID NO: 9 - protein, 197 aa, Pseudomonas
GKSRGMEVQFPYDQASIGHEGTLKVFTTRPDTLMGATYVAVAAEHPLATQAAQGNAALQAFIDECKSGSVAEADMATQEKKGMATSLFVEHPLTGEKLPVWVANYVLMHYGDGAVMAVPAHDERDFEFAHKYNLPVKAVVRTSAGDDVGSEWLAAYGEHGQLINSGEFDGLDFQGAFDAIEAALIRKDLGKSRTQFR

SEQ ID NO: 10 - protein, 188 aa, Staphylococcus aureus
GRSEGAKVSFDVDNTEGKVEVFTTRPDTIYGASFLVLSPEHALVNSITTDEYKEKVKAYQTEASKKSDLERTDLAKDKSGVFTGAYAINPLSGEKVQIWIADYVLSTYGTGAIMAVPAHDDRDYEFAKKFDLLIIEVIEGGNVEEAAYTGEGKHINSGELDGLENEAAITKAIQLLEQKGAGEKKVYK

SEQ ID NO: 11 - protein, 860 aa, Escherichia coli [sequence omitted]

SEQ ID NO: 12 - protein, 868 aa, Pseudomonas
MHEQYTPRDVEAAAQNAWDEQQSFAVTEQPGKETYYCLSMFPYPSGKLHMGHVRNYTIGDVIARYQRMLGKNVLQPMGWDAFGMPAENAAMKNNVAPAKWTYENIDYMKTQLKSLGLAIDWSREVTTCKPDYYRWEQWLFTRLFEKGVIYRKNGTVNWDPADQTVLANEQVIDGRGWRSGALIEKREIPMYYFRITDYADELLESLDELPGWPEQVKTMQRNWIGKSRGMEVQFPYDQASIGHEGTLKVFTTRPDTLMGATYVAVAAEHPLATQAAQGNAALQAFIDECKSGSVAEADMATQEKKGMATSLFVEHPLTGEKLPVWVANYVLMHYGDGAVMAVPAHDERDFEFAHKYNLPVKAVVRTSAGDDVGSEWLAAYGEHGQLINSGEFDGLDFQGAFDAIEAALIRKDLGKSRTQFRLRDWGISRQRYWGCPIPIIHCPSCGDVPVPEDQLPVTLPENVVPDGAGSPLARMPEFYECTCPKCGTAAKRETDTMDTFVESSWYFARYASPNYDKGLVDPKAANHWLPVDQYIGGIEHAILHLLYARFFHKLMRDEGLVTSNEPFKNLLTQGMVVAETYYRVASNGGKDWFNPADVEIERDAKAKIIGARLKTDGLPVEIGGTEKMSKSKNNGVDPQSMIEQYGADTCRLFMMFASPPDMSLEWSDSGVEGASRFLRRVWRLAQAHVAQGLPGQLDIAALSDEQKVIRRAIHAAIKQASTDVGQFHKFNTAIAQVMTVMNVLEKAPQVTAQDRALLQEGLEAVTLLLAPITPHISHELWKQLGHEQAVIDATWPSVDESALVQDTVTLVVQVNGKLRGQVEMPAAASREEIEAAARNNENVLRFTDGLTIRKVIVVPGKLVNIVAN

SEQ ID NO: 13 - protein, 804 aa, Staphylococcus aureus
MNYNHNQIEKKWQDYWDENKTFKTNDNLGQKKFYALDMFPYPSGAGLHVGHPEGYTATDIISRYKRMQGYNVLHPMGWDAFGLPAEQYALDTGNDPREFTKKNIQTFKRQIKELGFSYDWDREVNTTDPEYYKWTQWIFIQLYNKGLAYVDEVAVNWCPALGTVLSNEEVIDGVSERGGHPVYRKPMKQWVLKITEYADQLLADLDDLDWPESLKDMQRNWIGRSEGAKVSFDVDNTEGKVEVFTTRPDTIYGASFLVLSPEHALVNSITTDEYKEKVKAYQTEASKKSDLERTDLAKDKSGVFTGAYAINPLSGEKVQIWIADYVLSTYGTGAIMAVPAHDDRDYEFAKKFDLLIIEVIEGGNVEEAAYTGEGKHINSGELDGLENEAAITKAIQLLEQKGAGEKKVNYKLRDWLFSRQRYWGEPIPVIHWEDGTMTTVPEEELPLLLPETDEIKPSGTGESPLANIDSFVNVVDEKTGMKGRRETNTMPQWAGSCWYYLRYIDPKNENMLADPEKLKHWLPVDLYIGGVEHAVLHLLYARFWHKVLYDLGIVPTKEPFQKLFNQGMILGEGNEKMSKSKGNVINPDDIVQSHGADTLRLYEMFMGPLDAAIAWSEKGLDGSRRFLDRVWRLIVNEDGTLSSKIVTTNNKSLDKVYNQTVKKVTDDFETLGFNTAISQLMVFINECYKVDEVYKPYIEGFVKMLAPIAPHIGEELWSKLGHEESITYQPWPTYDEALLVDDEVEIVVQVNGKLRAKIKIAKDTSKEEMQEIALSNDNVKASIEGKDIMKVIAVPQKLVNIVAK

SEQ ID NO: 14 - DNA, 87 bp, E. coli
gcgaaggtggcggaattggtagacgcgctagcttcaggtgttagtgtccttacggacgtgggggttcaagtcccccccctcgcacca

SEQ ID NO: 15 - DNA, 85 bp, E. coli
gcgggagtggcgaaattggtagacgcaccagatttaggttctggcgccgcaaggtgtgcgagttcaagtctcgcctcccgcacca

SEQ ID NO: 16 - DNA, 85 bp, E. col
gccgaagtggcgaaatcggtagacgcagttgattcaaaatcaaccgtagaaatacgtgccggttcgagtccggccttcggcacca

SEQ ID NO: 17 - DNA, 87 bp, E. coli
gccgaggtggtggaattggtagacacgctaccttgaggtggtagtgcccaatagggcttacgggttcaagtcccgtcctcggtacca

SEQ ID NO: 18 - DNA, 87 bp, E. col
gcccggatggtggaatcggtagacacaagggatttaaaatccctcggcgttcgcgctgtgcgggttcaagtcccgctccgggtacca

SEQ ID NO: 19 - RNA, 91 nt, E. coli
  modified_base (8)..(8): s4u
gcccggauugguggaadcgggdagacacaagggayuuaaaaaaycccucggcguucgcgcugugcggguycaagucccgcuccggguacca

SEQ ID NO: 20 - RNA, 88 nt, E. coli
  modified_base (18)..(18): gm
gcgaagguggcggaaddggdagacgcgcuagcuucagugygyuaguguccuuacggacguggggguycaaguccccccccucgcacca

SEQ ID NO: 21 - RNA, 88 nt, E. coli
  modified_base (18)..(18): gm
gccgaggugguggaaddggdagacacgcuaccuugagugygguagugcccaauagggcuuacggguycaagucccguccucgguacca

SEQ ID NO: 22 - DNA, 85 bp, Pseudomonas aeruginosa
gcggacgtggtggaattggtagacacactggatttaggttccagcgccgcaaggcgtgagagttcgagtctctccgtccgcacca

SEQ ID NO: 23 - DNA, 89 bp, Staphylococcus aureus
gccggggtggcggaactggcagacgcacaggacttaaaatcctgcggtgagagatcaccgtaccggttcgattccggtcctcggcacca

SEQ ID NO: 24 - DNA, 89 bp, Staphylococcus aureus
gccggggtggcggaactggcagacgcacaggacttaaaatcctgcggtgagtgatcaccgtaccggttcgattccggtcctcggcacca

(19) United States
(12) Patent Application Publication
(10) Pub. No.: US 2011/0124597 A1
(43) Pub. Date: May 26, 2011

(54) BORON CONTAINING SMALL MOLECULES

(75) Inventors: Vincent S. HERNANDEZ, Watsonville, CA (US); Charles DING, San Mateo, CA (US); Jacob PLATTNER, Orinda, CA (US); Michael Richard Kevin ALLEY, Santa Clara, CA (US); Yong-Kang ZHANG, San Jose, CA (US); Yanchen ZHANG, Edmonton (CA)

(73) Assignee: Anacor Pharmaceuticals, Inc., Palo Alto, CA (US)

(21) Appl. No.: 12/873,036

(22) Filed: Aug. 31, 2010

Related U.S. Application Data
(60) Provisional application No. 61/246,029, filed on Sep. 25, 2009.

(51) Int. Cl. A61K 31/69; C07F 5/02; A01N 55/08; A61P 31/04; A01P 1/00; C12N 9/99

(52) U.S. Cl. 514/64; 544/229; 435/184

(57) ABSTRACT

This invention provides, among other things, novel compounds useful for treating bacterial infections, pharmaceutical compositions containing such compounds, as well as combinations of these compounds with at least one additional therapeutically effective agent.

### CROSS-REFERENCE TO RELATED APPLICATIONS

This application claims the benefit of U.S. Provisional Pat. App. No. 61/246,029, filed Sep. 25, 2009, which is incorporated by reference in its entirety for all purposes.

### SUMMARY OF THE INVENTION

This invention provides, among other things, novel compounds useful for treating bacterial infections, pharmaceutical compositions containing such compounds, as well as combinations of these compounds with at least one additional therapeutically effective agent.

### BRIEF DESCRIPTION OF THE DRAWINGS

FIG. 1 displays biological data for exemplary compounds of the invention.

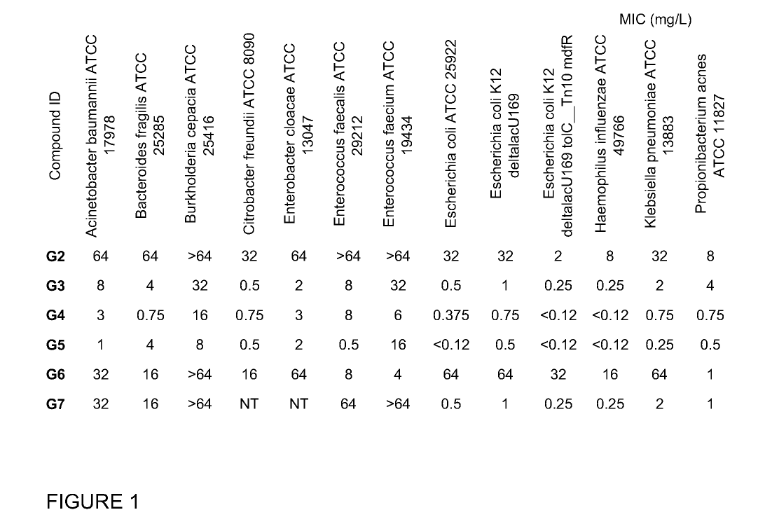

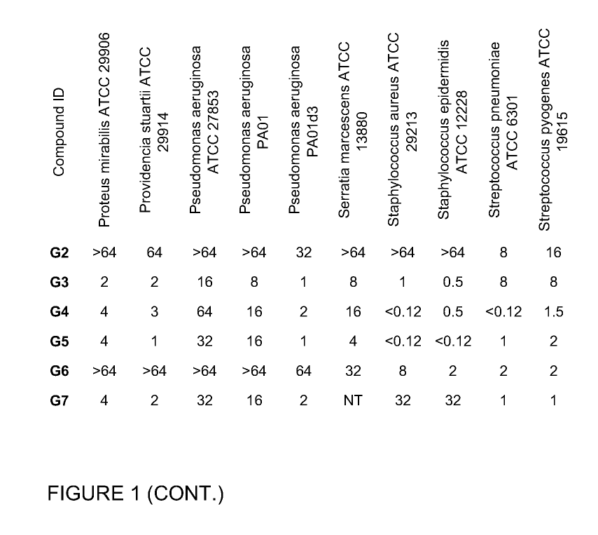

### DETAILED DESCRIPTION OF THE INVENTION

### I. Definitions and Abbreviations

As used herein, the singular forms “a,” “an”, and “the” include plural references unless the context clearly dictates otherwise. For example, reference to “an active agent” includes a single active agent as well as two or more different active agents in combination. It is to be understood that present teaching is not limited to the specific dosage forms, carriers, or the like, disclosed herein and as such may vary.

The abbreviations used herein generally have their conventional meaning within the chemical and biological arts.

The following abbreviations have been used: Ac is acetyl; AcOH is acetic acid; ACTBr is cetyltrimethylammonium bromide; AIBN is azobisisobutyronitrile or 2,2 azobisisobutyronitrile; aq. is aqueous; Ar is aryl; B₂pin₂ is bis(pinacolato)diboron; Bn is, in general, benzyl [see Cbz for one example of an exception]; (BnS)₂ is benzyl disulfide; BnSH is benzyl thiol or benzyl mercaptan; BnBr is benzyl bromide; Boc is tert-butoxy carbonyl; Boc₂O is di-tert-butyl dicarbonate; Bz is, in general, benzoyl; BzOOH is benzoyl peroxide; Cbz or Z is benzyloxycarbonyl or carboxybenzyl; Cs₂CO₃ is cesium carbonate; CSA is camphor sulfonic acid; CTAB is cetyltrimethylammonium bromide; Cy is cyclohexyl; DABCO is 1,4-diazabicyclo[2.2.2]octane; DCM is dichloromethane or methylene chloride; DHP is dihydropyran; DIAD is diisopropyl azodicarboxylate; DIEA or DIPEA is N,N-diisopropylethylamine; DMAP is 4-(dimethylamino)pyridine; DME is 1,2-dimethoxyethane; DMF is N,N-dimethylformamide; DMSO is dimethylsulfoxide; equiv or eq. is equivalent; EtOAc is ethyl acetate; EtOH is ethanol; Et₂O is diethyl ether; EDCI is N-(3-dimethylaminopropyl)-N′-ethylcarbodiimide hydrochloride; ELS is evaporative light scattering; equiv or eq is equivalent; h is hours; HATU is O-(7-azabenzotriazol-1-yl)-N,N,N′,N′-tetramethyluronium hexafluorophosphate; HOBt is N-hydroxybenzotriazole; HCl is hydrochloric acid; HPLC is high pressure liquid chromatography; ISCO Companion is automated flash chromatography equipment with fraction analysis by UV absorption available from Presearch; KOAc or AcOK is potassium acetate; K₂CO₃ is potassium carbonate; LiAlH₄ or LAH is lithium aluminum hydride; LDA is lithium diisopropylamide; LHMDS is lithium bis(trimethylsilyl)amide; KHMDS is potassium bis(trimethylsilyl)amide; LiOH is lithium hydroxide; m-CPBA is 3-chloroperoxybenzoic acid; MeCN or ACN is methyl cyanide or cyanomethane or ethanenitrile or acetonitrile which are all names for the same compound; MeOH is methanol; MgSO₄ is magnesium sulfate; mins or min is minutes; Mp or MP is melting point; NaCNBH₃ is sodium cyanoborohydride; NaOH is sodium hydroxide; Na₂SO₄ is sodium sulfate; NBS is N-bromosuccinimide; NH₄Cl is ammonium chloride; NIS is N-iodosuccinimide; N₂ is nitrogen; NMM is N-methylmorpholine; n-BuLi is n-butyllithium; overnight is O/N; PdCl₂(pddf) is 1,1′-Bis(diphenylphosphino)ferrocene]dichloropalladium(II); Pd/C is the catalyst known as palladium on carbon; Pd₂(dba)₃ is an organometallic catalyst known as tris(dibenzylideneacetone) dipalladium(0); RaNi or Raney Ni is Raney nickel; Ph is phenyl; PMB is p-methoxybenzyl; PrOH is 1-propanol; iPrOH is 2-propanol; POCl₃ is phosphorus chloride oxide; PTSA is para-toluene sulfonic acid; Pyr. or Pyr or Py as used herein means pyridine; RT or rt or r.t. is room temperature; sat. is saturated; Si-amine or Si—NH₂ is amino-functionalized silica, available from SiliCycle; Si-pyr is pyridyl-functionalized silica, available from SiliCycle; TEA or Et₃N is triethylamine; TFA is trifluoroacetic acid; Tf₂O is trifluoromethanesulfonic anhydride; THF is tetrahydrofuran; TFAA is trifluoroacetic anhydride; THP is tetrahydropyranyl; TMSI is trimethylsilyl iodide; H₂O is water; diNO₂PhSO₂Cl is dinitrophenyl sulfonyl chloride; 3-F-4-NO₂-PhSO₂Cl is 3-fluoro-4-nitrophenylsulfonyl chloride; 2-MeO-4-NO₂-PhSO₂Cl is 2-methoxy-4-nitrophenylsulfonyl chloride; and (EtO)₂POCH₂COOEt is a triethylester of phosphonoacetic acid known as triethyl phosphonoacetate.

“Compound of the invention,” as used herein refers to the compounds discussed herein, salts (e.g. pharmaceutically acceptable salts), prodrugs, solvates and hydrates of these compounds.

The term “poly” as used herein means at least 2. For example, a polyvalent metal ion is a metal ion having a valency of at least 2.

“Moiety” refers to a radical of a molecule that is attached to the remainder of the molecule.

The symbol [symbol] , whether utilized as a bond or displayed perpendicular to a bond, indicates the point at which the displayed moiety is attached to the remainder of the molecule.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). In some embodiments, the term “alkyl” means a straight or branched chain, or combinations thereof, which may be fully saturated, mono- or polyunsaturated and can include di- and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkylene” by itself or as part of another substituent means a divalent radical derived from an alkane, as exemplified, but not limited, by —CH₂CH₂CH₂CH₂—. Typically, an alkyl (or alkylene) group will have from 1 to 24 carbon atoms, with those groups having 10 or fewer carbon atoms being preferred in the invention. A “lower alkyl” or “lower alkylene” is a shorter chain alkyl or alkylene group, generally having eight or fewer carbon atoms.

The term “alkenylene” by itself or as part of another substituent means a divalent radical derived from an alkene.

The term “cycloalkylene” by itself or as part of another substituent means a divalent radical derived from a cycloalkyl.

The term “heteroalkylene” by itself or as part of another substituent means a divalent radical derived from an heteroalkane.

The term “heterocycloalkylene” by itself or as part of another substituent means a divalent radical derived from an heterocycloalkane.

The term “arylene” by itself or as part of another substituent means a divalent radical derived from an aryl.

The term “heteroarylene” by itself or as part of another substituent means a divalent radical derived from heteroaryl.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom. In some embodiments, the term “heteroalkyl,” by itself or in combination with another term, means a stable straight or branched chain, or combinations thereof, consisting of the stated number of carbon atoms and at least one heteroatom. In an exemplary embodiment, the heteroatoms can be selected from the group consisting of B, O, N and S, and wherein the nitrogen and sulfur atoms may optionally be oxidized and the nitrogen heteroatom may optionally be quaternized. The heteroatom(s) B, O, N and S may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —CH₂—CH═N—OCH₃, and —CH═CH—N(CH₃)—CH₃. Up to two heteroatoms may be consecutive, such as, for example, —CH₂—NH—OCH₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, substituent that can be a single ring or multiple rings (preferably from 1 or 2 or 3 rings), which are fused together or linked covalently. The term “heteroaryl” refers to aryl groups (or rings) that contain from one to four heteroatoms. In an exemplary embodiment, the heteroatom is selected from B, N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined herein. Thus, the term “arylalkyl” is meant to include those radicals in which an aryl group is attached to an alkyl group (e.g., benzyl, phenethyl, pyridiylethyl and the like) including those alkyl groups in which a carbon atom (e.g. a methylene group) has been replaced by, for example, an oxygen atom (e.g., phenoxymethyl, 2-pyridyloxymethyl, 3-(1-naphthyloxy)propyl, and the like).

For brevity, the term “heteroaryl” when used in combination with other terms (e.g., heteroaryloxy, heteroarylthioxy, heteroarylalkyl) includes those radicals in which a heteroaryl group is attached through the next moiety to the rest of the molecule. Thus, the term “heteroarylalkyl” is meant to include those radicals in which a heteroaryl group is attached to an alkyl group (e.g., pyridylmethyl and the like). The term “heteroaryloxy” is meant to include those radicals in which a heteroaryl group is attached to an oxygen atom. The term “heteroaryloxyalkyl” is meant to include those radicals in which an aryl group is attached to an oxygen atom which is then attached to an alkyl group. (e.g., 2-pyridyloxymethyl and the like).

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR′″″—C(NR′R″R′″)═NR′″, —NR″″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to (2m′+1), where m′ is the total number of carbon atoms in such radical. R′, R″, R′″, R″″ and R′″″ each preferably independently refer to hydrogen, substituted or unsubstituted heteroalkyl, substituted or unsubstituted aryl, e.g., aryl substituted with 1 or 2 or 3 halogens, substituted or unsubstituted alkyl, alkoxy or thioalkoxy groups, or arylalkyl groups. When a compound of the invention includes more than one R group, for example, each of the R groups is independently selected as are each R′, R″, R′″, R″″ and R′″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: —R′, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR′″″—C(NR′R″R′″)═NR″″, —NR″″—C(NR′R″)═NR′″, —S(O)R′, —S(O)₂R′, —S(O)₂NR′R″, —NR″SO₂R′, —CN, —NO₂, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, R″″ and R′″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″, R″″ and R′″″ groups when more than one of these groups is present.

Two of the substituents on adjacent atoms of the aryl or heteroaryl ring may optionally be replaced with a substituent of the formula -T-C(O)—(CRR′)_{q}-U-, wherein T and U are independently —NR—, —O—, —CRR′— or a single bond, and q is an integer from 0 or 1 or 2 or 3. Alternatively, two of the substituents on adjacent atoms of the aryl or heteroaryl ring may optionally be replaced with a substituent of the formula -A-(CH₂)ᵣ—B-, wherein A and B are independently —CRR′—, —O—, —NR—, —S—, —S(O)—, —S(O)₂—, —S(O)₂NR′— or a single bond, and r is an integer from 1 or 2 or 3 or 4. One of the single bonds of the new ring so formed may optionally be replaced with a double bond. Alternatively, two of the substituents on adjacent atoms of the aryl or heteroaryl ring may optionally be replaced with a substituent of the formula (CRR′)ₛ—X—(CR″R′″)_{d}—, where s and d are independently integers from 0 or 1 or 2 or 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁ or C₂ or C₃ or C₄ or C₅ or C₆)alkyl.

“Ring” as used herein, means a substituted or unsubstituted cycloalkyl, substituted or unsubstituted heterocycloalkyl, substituted or unsubstituted aryl, or substituted or unsubstituted heteroaryl. A ring includes fused ring moieties. The number of atoms in a ring is typically defined by the number of members in the ring. For example, a “5- to 7-membered ring” means there are 5 or 6 or 7 atoms in the encircling arrangement. Unless otherwise specified, the ring optionally includes a heteroatom. Thus, the term “5- to 7-membered ring” includes, for example phenyl, pyridinyl and piperidinyl. The term “5- to 7-membered heterocycloalkyl ring”, on the other hand, would include pyridinyl and piperidinyl, but not phenyl. The term “ring” further includes a ring system comprising more than one “ring”, wherein each “ring” is independently defined as above.

As used herein, the term “heteroatom” includes atoms other than carbon (C) and hydrogen (H). Examples include oxygen (O), nitrogen (N) sulfur (S), silicon (Si), and boron (B).

The term “leaving group” means a functional group or atom which can be displaced by another functional group or atom in a substitution reaction, such as a nucleophilic substitution reaction. By way of example, representative leaving groups include triflate, chloro, bromo and iodo groups; sulfonic ester groups, such as mesylate, tosylate, brosylate, nosylate and the like; and acyloxy groups, such as acetoxy, trifluoroacetoxy and the like.

The symbol “R” is a general abbreviation that represents a substituent group that is selected from substituted or unsubstituted alkyl, substituted or unsubstituted heteroalkyl, substituted or unsubstituted aryl, substituted or unsubstituted heteroaryl, substituted or unsubstituted cycloalkyl and substituted or unsubstituted heterocycloalkyl groups.

By “effective” amount of a drug, formulation, or permeant is meant a sufficient amount of an active agent to provide the desired local or systemic effect. A “Topically effective,” “pharmaceutically effective,” or “therapeutically effective” amount refers to the amount of drug needed to effect the desired therapeutic result.

The term “pharmaceutically acceptable salt” is meant to include a salt of a compound of the invention which are prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino (such as choline or diethylamine or amino acids such as d-arginine, l-arginine, d-lysine, or l-lysine), or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the invention provides compounds which are in a prodrug form. Prodrugs of the compounds described herein readily undergo chemical changes under physiological conditions to provide the compounds of the invention. Additionally, prodrugs can be converted to the compounds of the invention by chemical or biochemical methods in an ex vivo environment.

Certain compounds of the invention can exist in unsolvated forms as well as solvated forms, including hydrated forms. In general, the solvated forms are equivalent to unsolvated forms and are encompassed within the scope of the invention. Certain compounds of the invention may exist in multiple crystalline or amorphous forms.

Certain compounds of the invention possess asymmetric carbon atoms (optical centers) or double bonds; the racemates, diastereomers, geometric isomers and individual isomers are encompassed within the scope of the invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers. Likewise, all tautomeric forms are included.

Compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

The compounds of the invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

The term “excipients” is conventionally known to mean carriers, diluents and/or vehicles used in formulating drug compositions effective for the desired use.

The term “microbial infection” or “infection by a microorganism” refers to any infection of a host tissue by an infectious agent including, but not limited to, bacteria or protozoa (see, e.g., Harrison's Principles of Internal Medicine, pp. 93-98 (Wilson et al., eds., 12th ed. 1991); Williams et al., J. of Medicinal Chem. 42: 1481-1485 (1999), herein each incorporated by reference in their entirety).

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Inhibiting” and “blocking,” are used interchangeably herein to refer to the partial or full blockade of enzyme. In an exemplary embodiment, the enzyme is an editing domain of a tRNA synthetase.

Boron is able to form additional covalent or dative bonds with oxygen, sulfur or nitrogen under some circumstances in this invention.

Embodiments of the invention also encompass compounds that are poly- or multi-valent species, including, for example, species such as dimers, trimers, tetramers and higher homologs of the compounds of use in the invention or reactive analogues thereof.

“Salt counterion”, as used herein, refers to positively charged ions that associate with a compound of the invention when the boron is fully negatively or partially negatively charged. Examples of salt counterions include H⁺, H₃O⁺, ammonium, potassium, calcium, magnesium, organic amino (such as choline or diethylamine or amino acids such as d-arginine, l-arginine, d-lysine, or l-lysine) and sodium.

The compounds comprising a boron bonded to a carbon and three heteroatoms (such as three oxygens described in this section) can optionally contain a fully negatively charged boron or partially negatively charged boron. Due to the negative charge, a positively charged counterion may associate with this compound, thus forming a salt. Examples of positively charged counterions include H⁺, H₃O⁺, ammonium, potassium, calcium, magnesium, organic amino (such as choline or diethylamine or amino acids such as d-arginine, l-arginine, d-lysine, l-lysine), and sodium. These salts of the compounds are implicitly contained in descriptions of these compounds.

### II. Introduction

The invention provides novel boron compounds.

### III. The Compounds

### III.a) Cyclic Boronic Esters

In one aspect, the invention provides a compound of the invention. In an exemplary embodiment, the invention provides a compound described herein.

In another aspect, the invention provides a compound having a structure according to the formula:

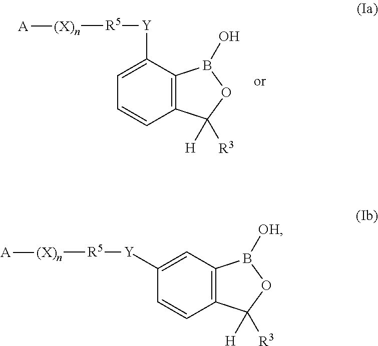

collectively referred to herein as Formula I, wherein Y is O or S; R⁵ is substituted or unsubstituted alkylene or substituted or unsubstituted heteroalkylene; R³ is selected from the group consisting of H, cyano, substituted or unsubstituted nitroalkyl and substituted or unsubstituted aminoalkyl; A is an antibiotic or its pharmacophore; n is 0 or 1 or 2 or 3 or 4 or 5; each X is a linker, or a salt thereof.

In an exemplary embodiment, Y is O. In an exemplary embodiment, Y is S.

In an exemplary embodiment, R⁵ is substituted heteroalkylene. In an exemplary embodiment, R⁵ is unsubstituted heteroalkylene. In an exemplary embodiment, R⁵ is substituted alkylene. In an exemplary embodiment, R⁵ is unsubstituted alkylene. In an exemplary embodiment, R⁵ is:

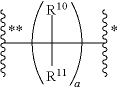

wherein [symbol] connotes a covalent attachment to Y. [symbol] connotes a covalent attachment to A, when n is 0, or X, when n is not 0. The index a is 1 or 2 or 3 or 4 or 5 or 6 or 7 or 8 or 9 or 10. Each R¹⁰ and each R¹¹ are each the same or different and each are selected from the group consisting of H, substituted or unsubstituted alkyl, OH and NH₂. In an exemplary embodiment, the index a is 1 or 2 or 3 or 4 or 5 or 6 or 7 or 8. In an exemplary embodiment, the index a is 2 or 3 or 4. In an exemplary embodiment, each R¹⁰ and each R¹¹ are each the same or different and each are selected from the group consisting of H, substituted or unsubstituted alkyl, OH and NH₂. In an exemplary embodiment, each R¹⁰ and each R¹¹ are each the same or different and each are selected from the group consisting of H, hydroxyalkyl and NH₂. In an exemplary embodiment, at least one R¹⁰ or R¹¹ is hydroxyalkyl or NH₂. In an exemplary embodiment, each R¹⁰ and each R¹¹ is H.

In an exemplary embodiment, R³ is —(CR²⁰R²¹)ₙNR²²R²³ in which the index n is 1 or 2 or 3 or 4 or 5 or 6 or 7 or 8 or 9 or 10; each R²⁰ and each R²¹ are each the same or different and each are selected from the group consisting of H, R²⁶, OR²⁶, NR²⁶R²⁷, SR²⁶, —S(O)R²⁶, —S(O)₂R²⁶, —S(O)₂NR²⁶R²⁷, —C(O)R²⁷, —C(O)OR²⁷, —C(O)NR²⁶R²⁷; each R²² and each R²³ are each the same or different and each are selected from the group consisting of H, —S(O)R²⁸, —S(O)₂R²⁸, —S(O)₂NR²⁸R²⁹, —C(O)R²⁸, —C(O)OR²⁸, —C(O)NR²⁸R²⁹, nitro, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl wherein each R²⁶, each R²⁷, each R²⁸ and each R²⁹ are each the same or different and each are selected from the group consisting of H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, n is 1 or 2 or 3 or 4 or 5. In an exemplary embodiment, n is 1. In an exemplary embodiment, R²⁰ is substituted or unsubstituted alkyl. In an exemplary embodiment, R²⁰ is unsubstituted alkyl. In an exemplary embodiment, R²⁰ is C₁ or C₂ or C₃ or C₄ unsubstituted alkyl. In an exemplary embodiment, R²⁰ is methyl. In an exemplary embodiment, R²¹ is H. In an exemplary embodiment, R²³ is H. In an exemplary embodiment, R³ is cyano or —CH₂NO₂. In an exemplary embodiment, R²² is —C(O)R²⁸ or —C(O)OR²⁸. In an exemplary embodiment, R²⁸ is selected from the group consisting of substituted or unsubstituted alkyl, substituted or unsubstituted heteroalkyl and substituted or unsubstituted aryl. In an exemplary embodiment, R²⁸ is —(CR³⁰R³¹)ₘR³², wherein R³² is selected from the group consisting of substituted or unsubstituted aryl, —NR³³R³⁴ and OR³³, wherein the index m is 0 or 1 or 2 or 3 or 4 or 5 or 6 or 7 or 8 or 9 or 10; each R³³ and each R³⁴ are each the same or different and each are selected from the group consisting of H, nitro, halogen, cyano, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In an exemplary embodiment, R²⁸ is selected from the group consisting of

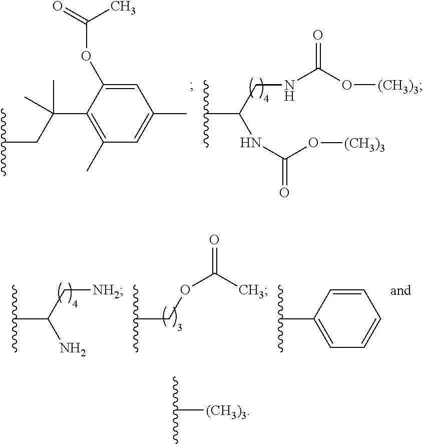

In an exemplary embodiment, X is —(C₁ or C₂ or C₃ or C₄ or C₅ or C₆)alkylene. In an exemplary embodiment, X is —(C₁ or C₂ or C₃ or C₄ or C₅ or C₆)alkenylene. In an exemplary embodiment, X is (C₁ or C₂ or C₃ or C₄ or C₅ or C₆)alkynylene. In an exemplary embodiment, X is —(C₃ or C₄ or C₅ or C₆)cycloalkylene. In an exemplary embodiment, X is —O—. In an exemplary embodiment, X is —C(H)═—. In an exemplary embodiment, X is —C(═O)—. In an exemplary embodiment, X is —C(═N—O—R¹³)—, wherein R¹³ is selected from the group consisting of hydrogen, (C₁ or C₂ or C₃ or C₄ or C₅ or C₆)alkyl and substituted (C₁ or C₂ or C₃ or C₄ or C₅ or C₆)alkyl, i) —S(O)ₙ—, wherein n is 0 or 1 or 2. In an exemplary embodiment, X is —N(R¹⁴)—, wherein R¹⁴ is selected from the group consisting of hydrogen, (C₁ or C₂ or C₃ or C₄ or C₅ or C₆)alkyl and substituted (C₁ or C₂ or C₃ or C₄ or C₅ or C₆)alkyl. In an exemplary embodiment, X is arylene. In an exemplary embodiment, X is heteroarylene. In an exemplary embodiment, X is heterocycloalkylene.

In an exemplary embodiment, n is not zero, and X is:

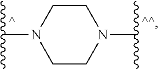

where [symbol] is a covalent attachment to A, and [symbol] is a covalent attachment to R⁵.

In an exemplary embodiment, n is zero. In an exemplary embodiment, n is 1.

In an exemplary embodiment, A is an antibiotic. In an exemplary embodiment, A is a pharmacophore of an antibiotic.

In an exemplary embodiment, A is an oxazolidinone antibiotic. In an exemplary embodiment, A is a pharmacophore of an oxazolidinone antibiotic. In an exemplary embodiment, A comprises an oxazolidinone. In an exemplary embodiment, A comprises an oxazolidinone, and the oxazolidinone is selected from the group consisting of linezolid, torezolid, posizolid, ranbezolid, radezolid and eperezolid. In an exemplary embodiment, A comprises linezolid. In an exemplary embodiment, A is:

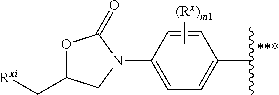

wherein [symbol] is a covalent attachment to X when n is not zero, and to R⁵ when n is zero. R^{x} is halogen. R^{xi} is CH₃CONH—, OR¹⁰ or (un)substituted triazole wherein R¹⁰ is selected from the group consisting of H, unsubstituted heteroaryl and unsubstituted alkyl; and m1 is 0 or 1 or 2 or 3.

In an exemplary embodiment, A is selected from the group consisting of:

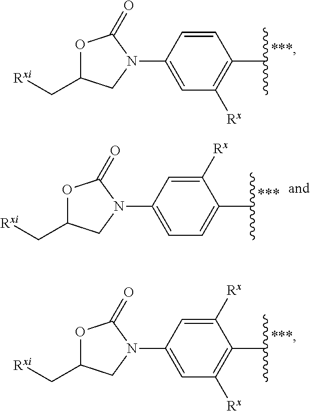

wherein R^{xi} is as described herein, and each R^{x} are each the same or different and each are selected from F or Cl or Br or I. In another exemplary embodiment, each halogen are each the same or different and each are selected from F or Cl.

In an exemplary embodiment, A is selected from the group consisting of:

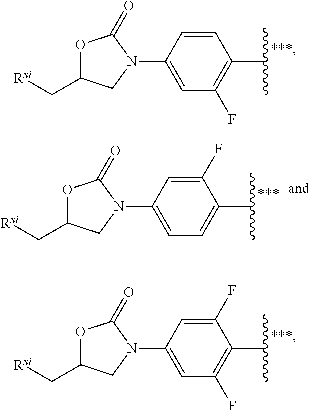

wherein R^{xi} is as described herein.

In an exemplary embodiment, A is:

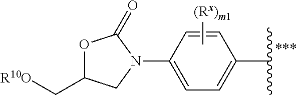

wherein R^{x}, m1 and R¹⁰ are as described herein.

In an exemplary embodiment, A is:

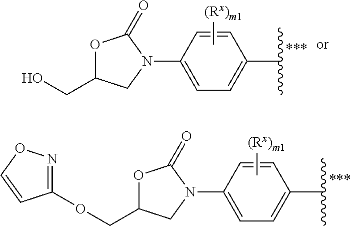

wherein R^{x} and m1 are as described herein.

In an exemplary embodiment, A is:

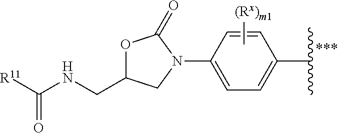

wherein R^{x} and m1 are as described herein, R¹¹ is unsubstituted alkyl.

In an exemplary embodiment, A is:

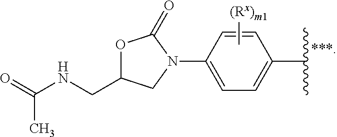

wherein R^{x} and m1 are as described herein.

In an exemplary embodiment, A is selected from the group consisting of:

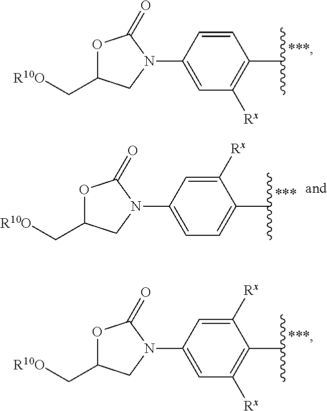

wherein R¹⁰ is as described herein, and each R^{x} are each the same or different and each are selected from the group consisting of F, Cl, Br and I. In another exemplary embodiment, each R^{x} is F or Cl.

In an exemplary embodiment, A is selected from the group consisting of:

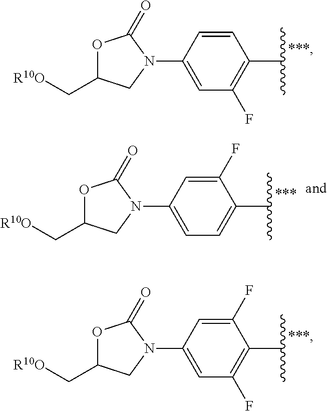

wherein R¹⁰ is as described herein.

In an exemplary embodiment, A is selected from the group consisting of:

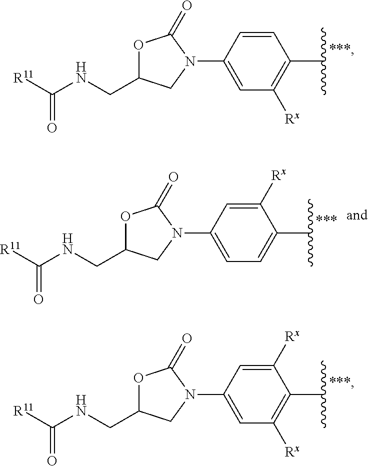

wherein R¹¹ is as described herein, and each R^{x} are each the same or different and each are selected from the group consisting of F, Cl, Br and I. In another exemplary embodiment, each R^{x} is F or Cl. In an exemplary embodiment, R¹¹ is methyl.

In an exemplary embodiment, A is selected from the group consisting of:

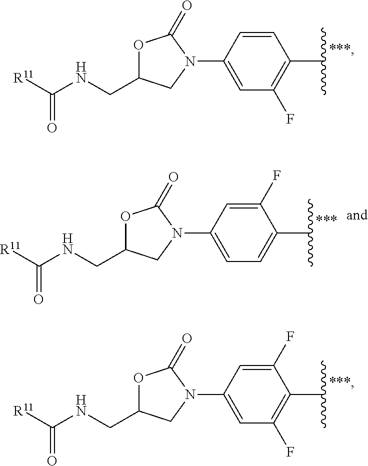

wherein R¹¹ is as described herein. In an exemplary embodiment, R¹¹ is methyl.

In an exemplary embodiment, A is selected from the group consisting of:

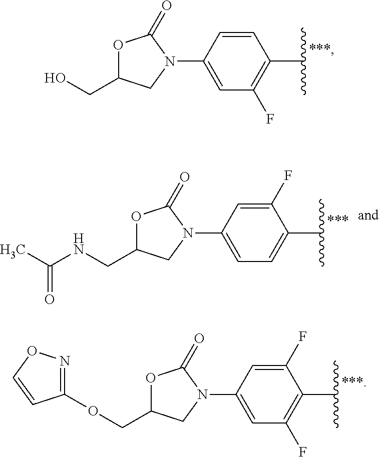

In an exemplary embodiment, A is selected from the group consisting of:

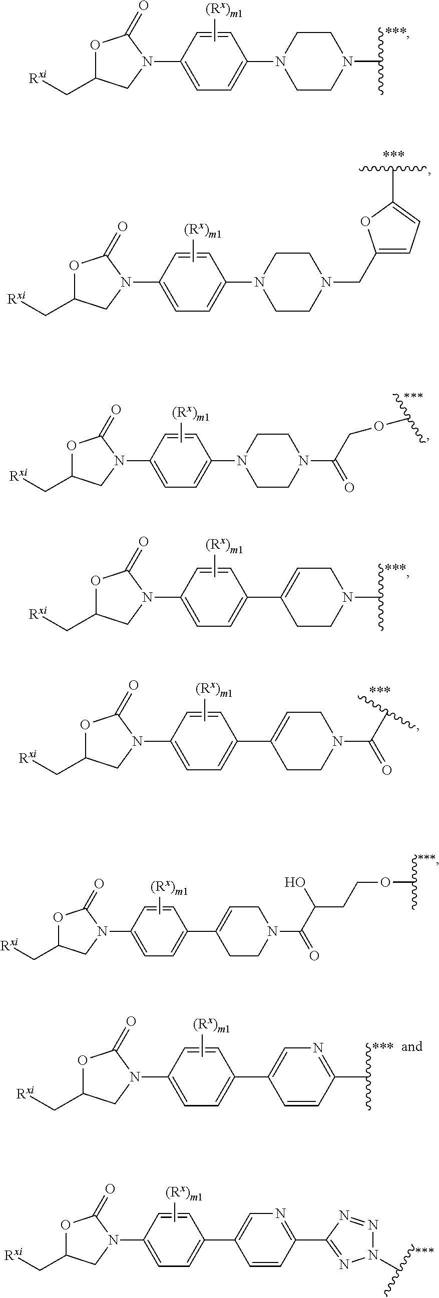

wherein R^{xi} and R^{x} and m1 are as described herein.

In an exemplary embodiment, A is selected from the group consisting of:

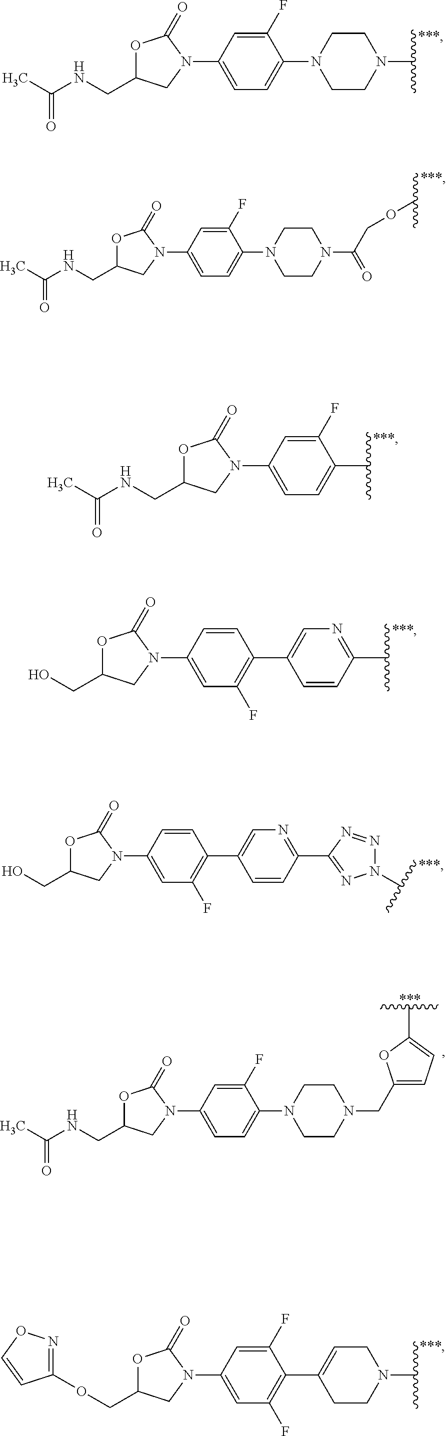

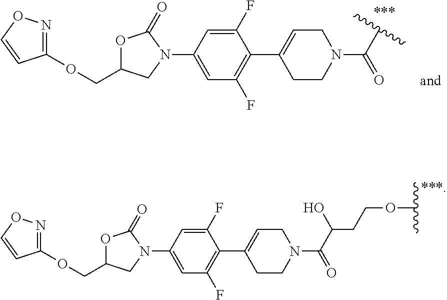

In an exemplary embodiment, A is a quinolone antibiotic. In an exemplary embodiment, A is a pharmacophore of a quinolone antibiotic. In an exemplary embodiment, A comprises a quinolone. In an exemplary embodiment, A comprises nalidixic acid or oxolinic acid or piromidic acid or pipemidic acid or cinoxacin or flumequine. In an exemplary embodiment, A comprises ciprofloxacin or enoxacin or fleroxacin or lomefloxacin or nadifloxacin or norfloxacin or ofloxacin or pefloxacin or rufloxacin. In an exemplary embodiment, A comprises ciprofloxacin. In an exemplary embodiment, A comprises balofloxacin or gatifloxacin or grepafloxacin or levofloxacin or moxifloxacin or pazufloxacin or sparfloxacin or temafloxacin or tosufloxacin. In an exemplary embodiment, A comprises clinafloxacin or besifloxacin or gemifloxacin or sitafloxacin or alatrofloxacin or trovafloxacin or prulifloxacin. In an exemplary embodiment, A comprises garenoxacin or delafloxacin. In an exemplary embodiment, A comprises danofloxacin or difloxacin or enrofloxacin or ibafloxacin or marbofloxacin or rosoxacin or orbifloxacin or sarafloxacin.

In an exemplary embodiment, the compound has a structure which is selected from the group consisting of

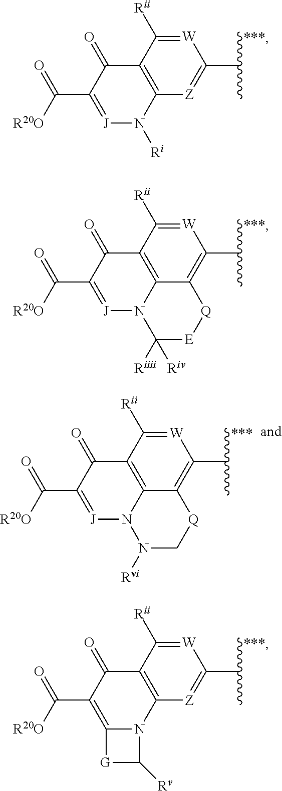

wherein [symbol] is a covalent attachment to X when n is not zero, and to R⁵ when n is zero; R²⁰ is selected from the group consisting of H, unsubstituted alkyl, benzyl and negative charge; Rⁱ is selected from the group consisting of (C₁ or C₂ or C₃ or C₄ or C₅ or C₆)alkyl, halosubstituted (C₁ or C₂ or C₃ or C₄ or C₅ or C₆)alkyl, (C₃ or C₄ or C₅ or C₆)cycloalkyl, halosubstituted (C₃ or C₄ or C₅ or C₆)cycloalkyl, aryl, substituted aryl, heteroaryl, and halo-substituted heteroaryl. Rⁱⁱ is selected from the group consisting of hydrogen, halogen, amino and unsubstituted alkyl; each and each Rⁱⁱⁱ is hydrogen or (C₁ or C₂ or C₃ or C₄ or C₅ or C₆)alkyl; W is selected from the group consisting of CH, CF, and N; Z is selected from the group consisting of N, CN, CH, C—F, C—Cl and CR^{vii}. R^{vii} is unsubstituted alkyl or halosubstituted alkyl or unsubstituted alkoxy or halosubstituted alkoxy. Q is CH₂ or O or S. E is CH₂ or O or S. G is O or S. R^{v} is unsubstituted alkyl. R^{vi} is unsubstituted alkyl. J is —CH or NH.

In an exemplary embodiment, Z is CR^{vii}, and R^{vii} is selected from the group consisting of CF₃, CH₃, OCH₃, OCH₂F and OCHF₂.

In an exemplary embodiment, A is selected from the group consisting of:

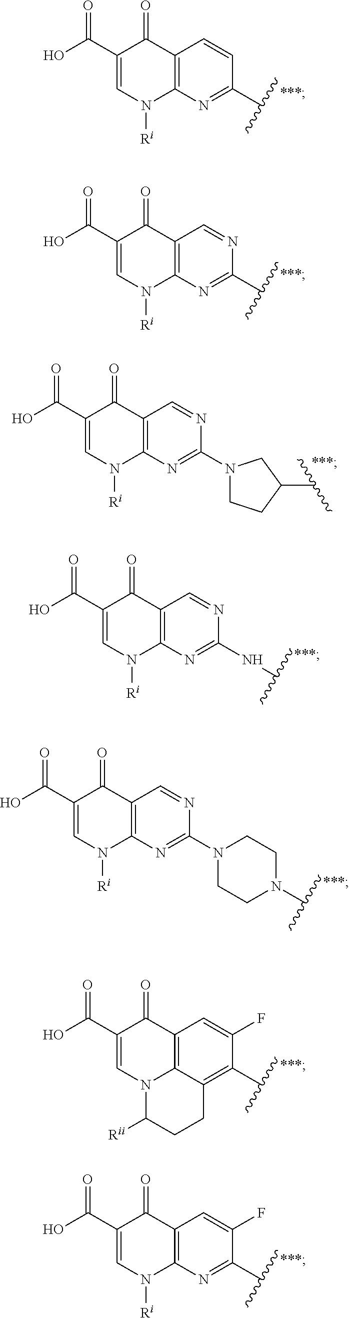

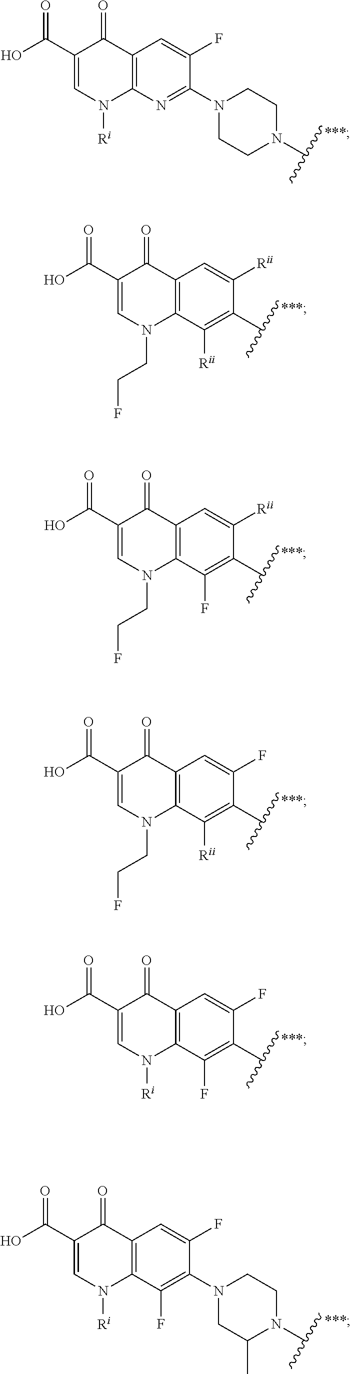

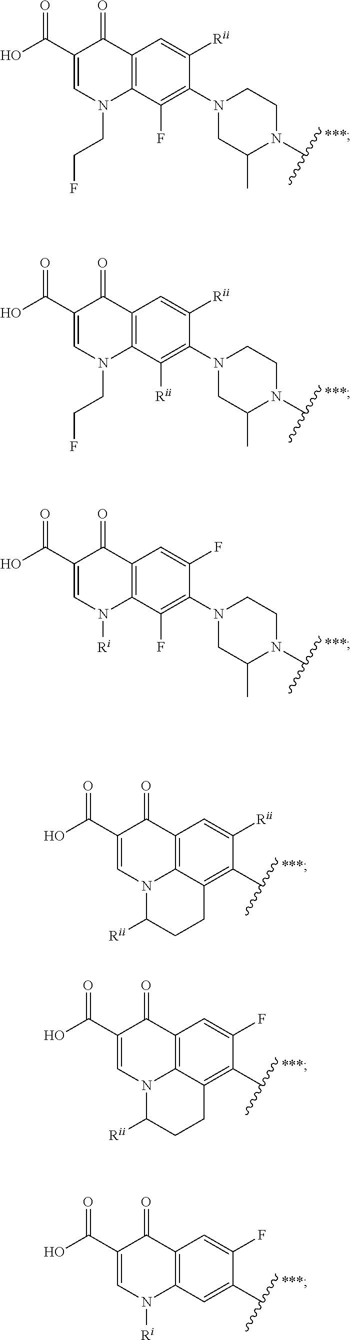

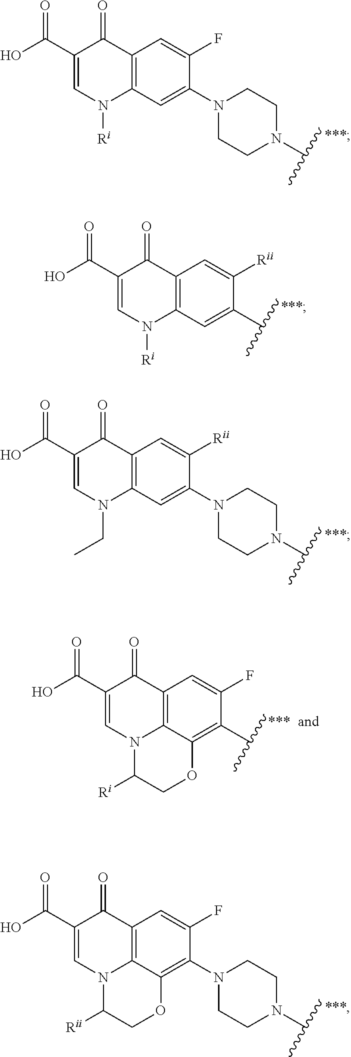

wherein Rⁱ is as described herein and Rⁱⁱ is halogen.

In an exemplary embodiment, A is selected from the group consisting of:

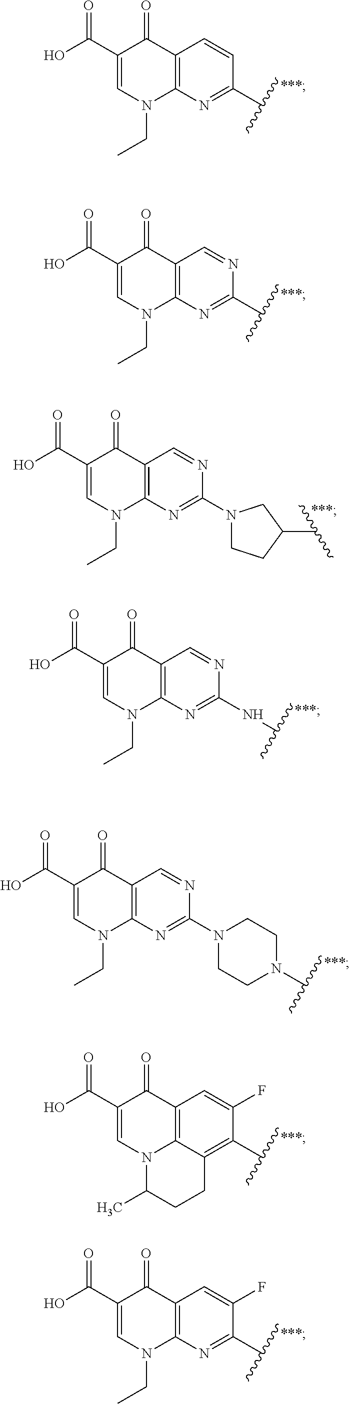

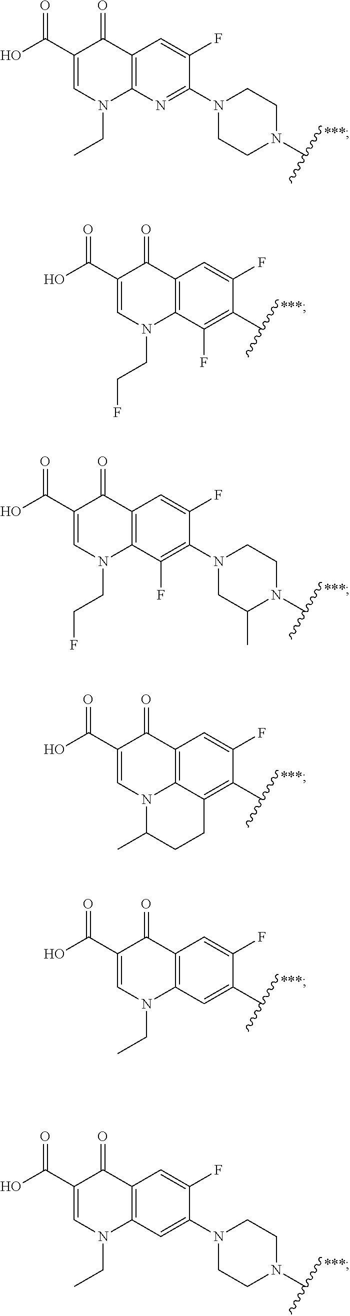

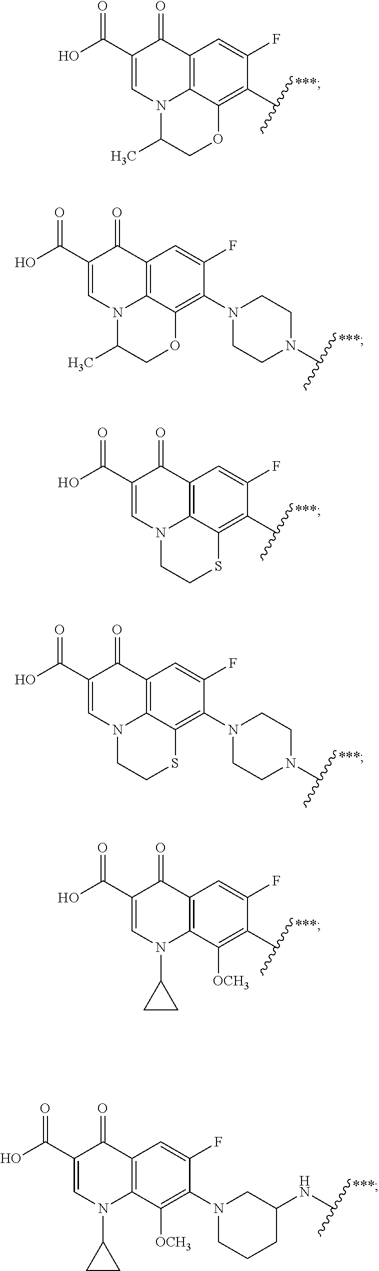

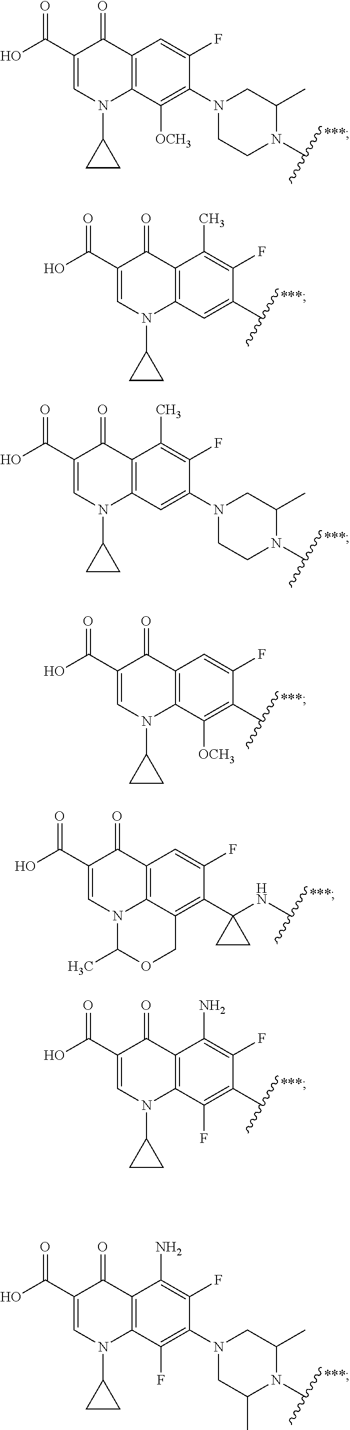

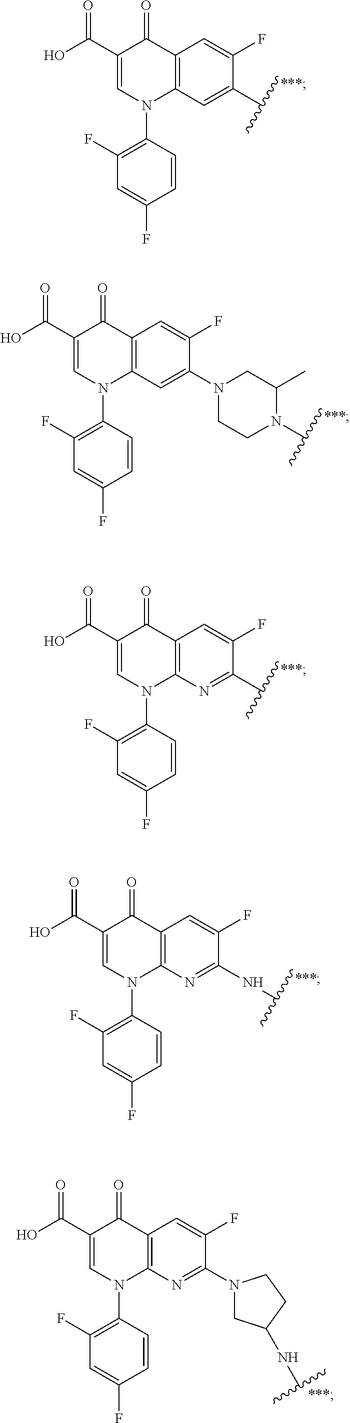

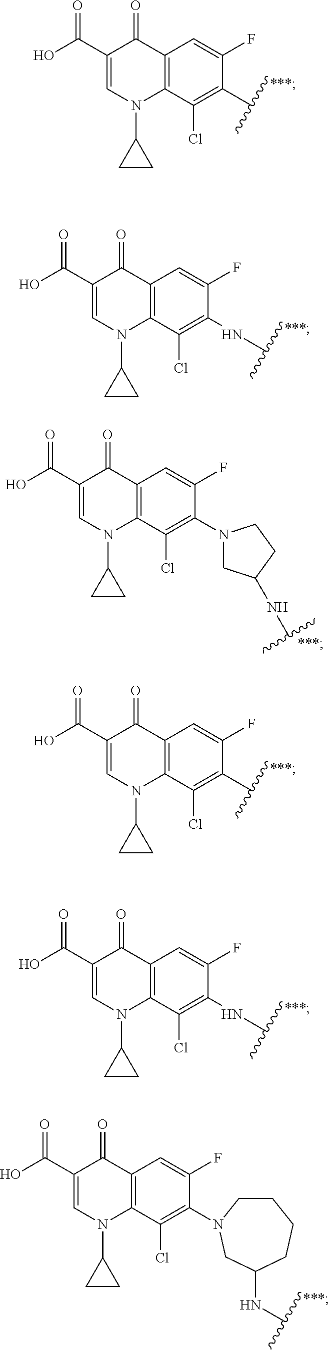

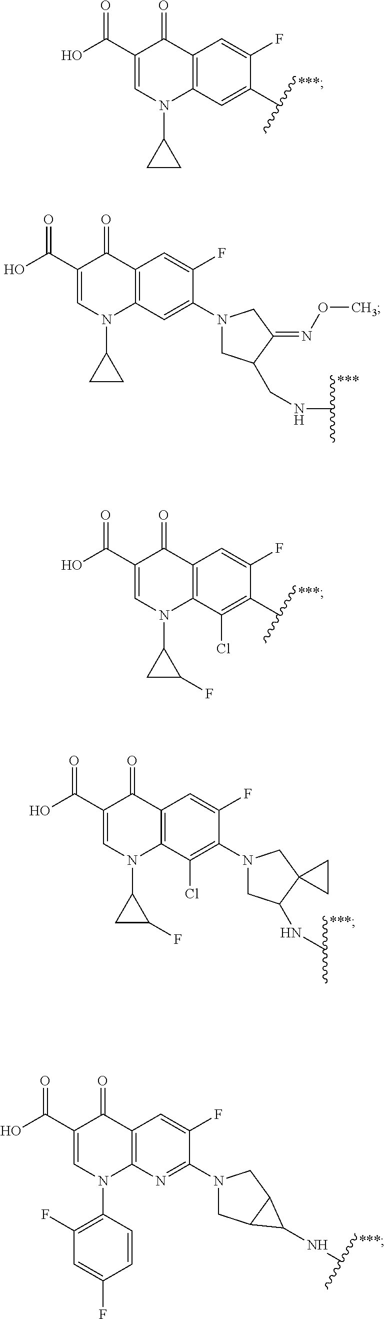

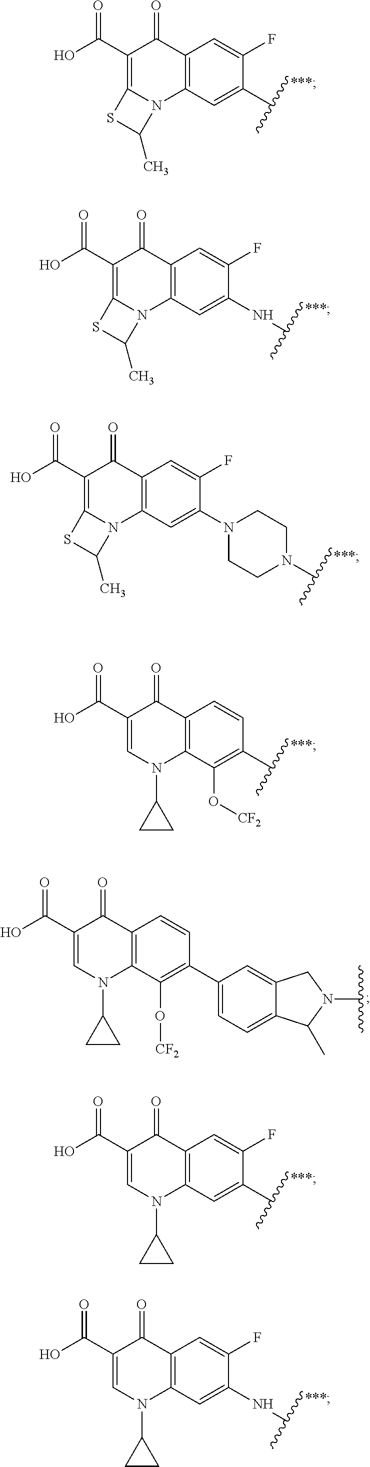

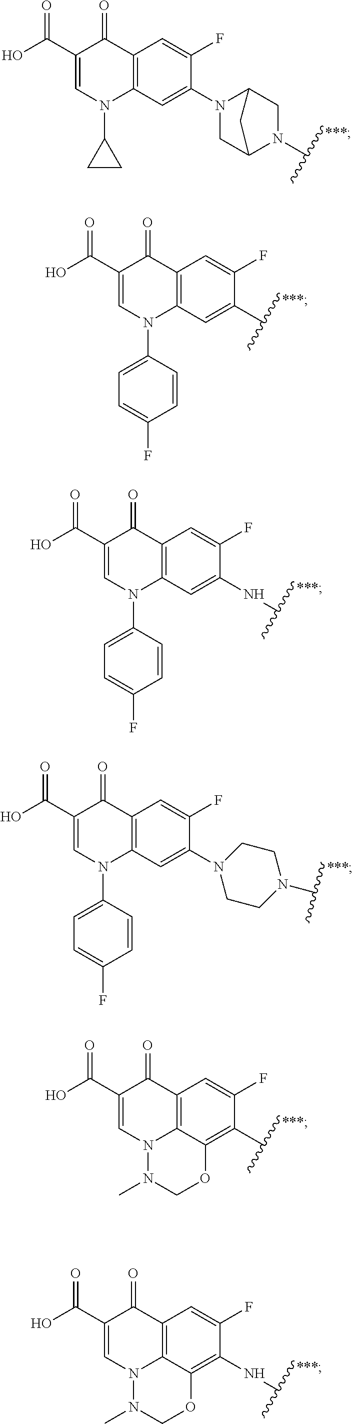

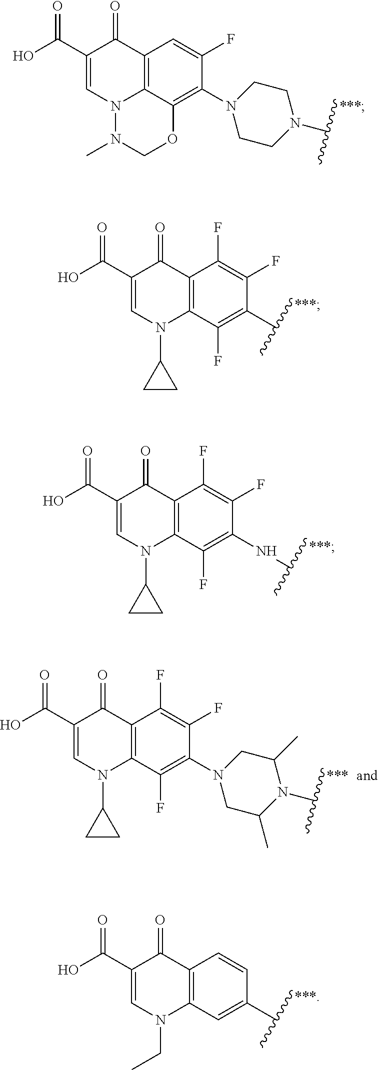

In an exemplary embodiment, the compound has a structure which is

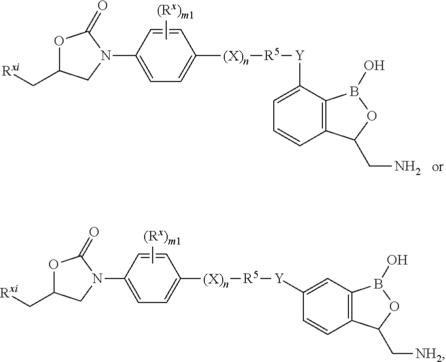

wherein R^{xi}, R^{x}, X, R⁵, Y, m1 and n are as described herein.

In an exemplary embodiment, the compound has a structure which is

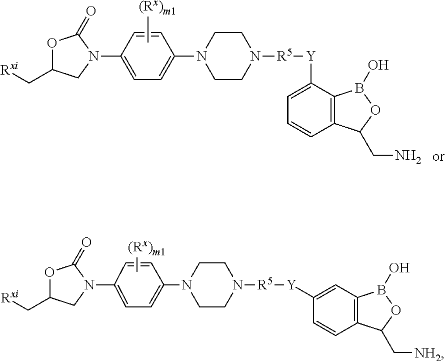

wherein R^{xi}, R^{x}, R⁵, Y and m1 are as described herein.

In an exemplary embodiment, the compound has a structure which is

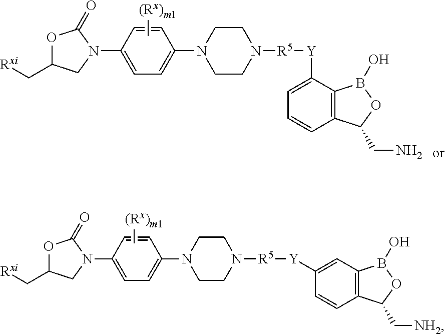

wherein R^{xi}, R^{x}, R⁵, Y and m1 are as described herein.

In an exemplary embodiment, the compound has a structure which is

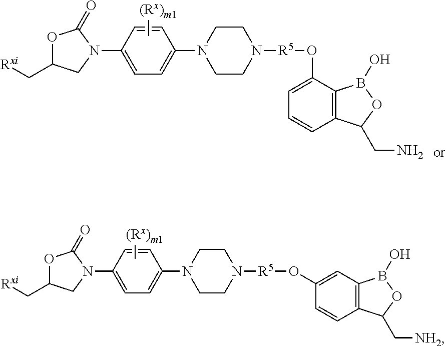

wherein R^{xi}, R^{x}, R⁵ and m1 are as described herein.

In an exemplary embodiment, the compound has a structure which is

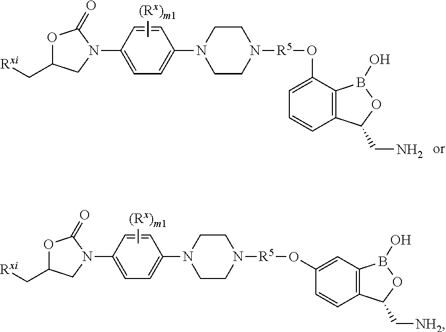

wherein R^{xi}, R^{x}, R⁵ and m1 are as described herein.

In an exemplary embodiment, the compound has a structure which is

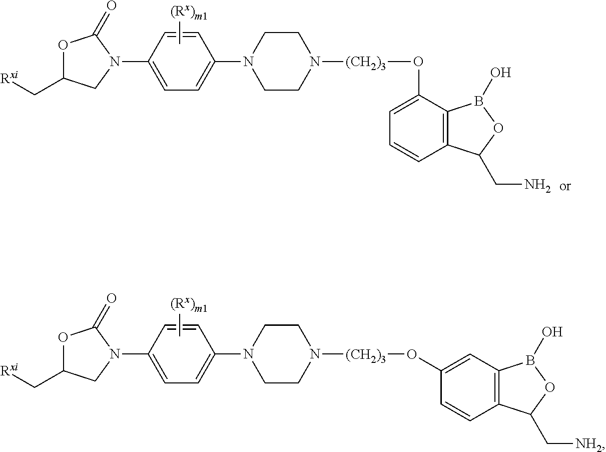

wherein R^{xi}, R^{x} and m1 are as described herein.

In an exemplary embodiment, the compound has a structure which is

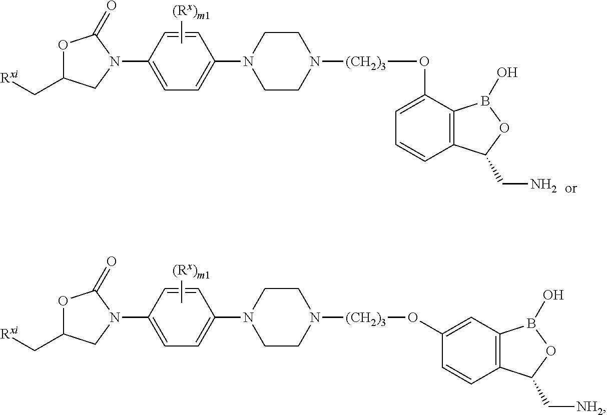

wherein R^{xi}, R^{x} and m1 are as described herein.

In an exemplary embodiment, the compound has a structure which is

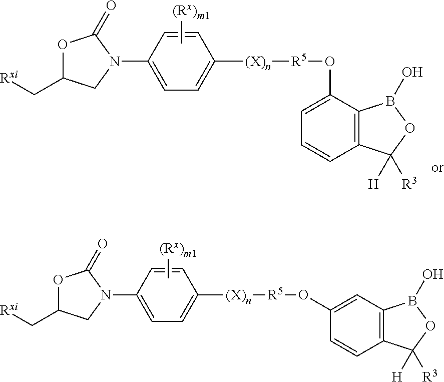

and R⁵ is unsubstituted C₁-C₆ alkylene. In an exemplary embodiment, R⁵ is methylene. In an exemplary embodiment, R⁵ is ethylene. In an exemplary embodiment, R⁵ is unsubstituted C₃ alkylene. In an exemplary embodiment, R⁵ is unsubstituted C₄ alkylene. In an exemplary embodiment, R⁵ is unsubstituted C₅ alkylene. In an exemplary embodiment, R⁵ is unsubstituted C₆ alkylene.

In an exemplary embodiment, the compound has a structure which is

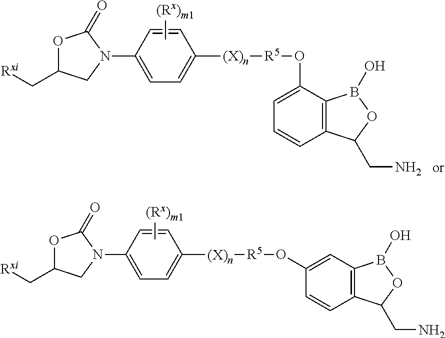

and R⁵ is unsubstituted C₁-C₆ alkylene. In an exemplary embodiment, R⁵ is methylene. In an exemplary embodiment, R⁵ is ethylene. In an exemplary embodiment, R⁵ is unsubstituted C₃ alkylene. In an exemplary embodiment, R⁵ is unsubstituted C₄ alkylene. In an exemplary embodiment, R⁵ is unsubstituted C₅ alkylene. In an exemplary embodiment, R⁵ is unsubstituted C₆ alkylene.

In an exemplary embodiment, the compound has a structure which is

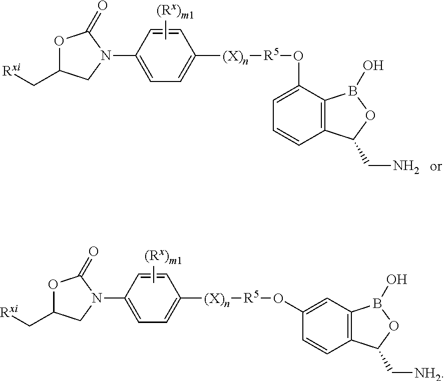

and R⁵ is unsubstituted C₁-C₆ alkylene. In an exemplary embodiment, R⁵ is methylene. In an exemplary embodiment, R⁵ is ethylene. In an exemplary embodiment, R⁵ is unsubstituted C₃ alkylene. In an exemplary embodiment, R⁵ is unsubstituted C₄ alkylene. In an exemplary embodiment, R⁵ is unsubstituted C₅ alkylene. In an exemplary embodiment, R⁵ is unsubstituted C₆ alkylene.

In an exemplary embodiment, the compound has a structure which is

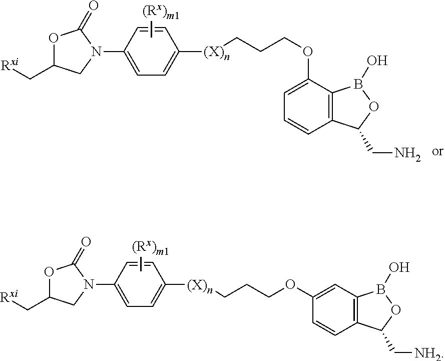

and R⁵ is unsubstituted C₁-C₆ alkylene. In an exemplary embodiment, R⁵ is methylene. In an exemplary embodiment, R⁵ is ethylene. In an exemplary embodiment, R⁵ is unsubstituted C₃ alkylene. In an exemplary embodiment, R⁵ is unsubstituted C₄ alkylene. In an exemplary embodiment, R⁵ is unsubstituted C₅ alkylene. In an exemplary embodiment, R⁵ is unsubstituted C₆ alkylene.

In an exemplary embodiment, the compound has a structure which is

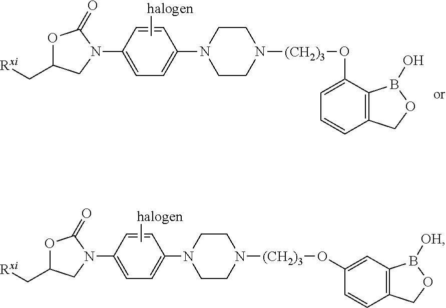

and R^{xi} is as described herein.

In an exemplary embodiment, the compound has a structure which is

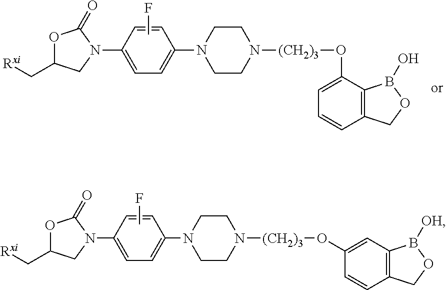

and R^{xi} is as described herein.

In an exemplary embodiment, the compound has a structure which is

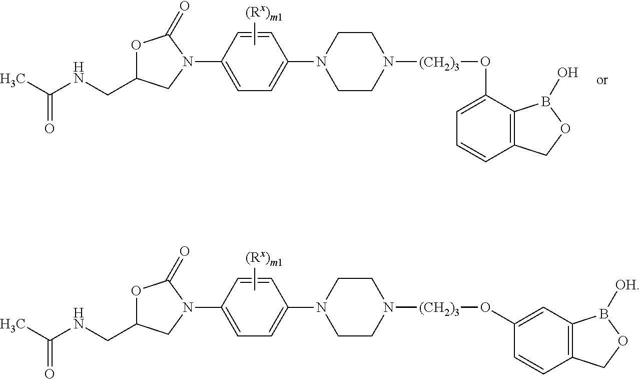

In an exemplary embodiment, the compound has a structure which is

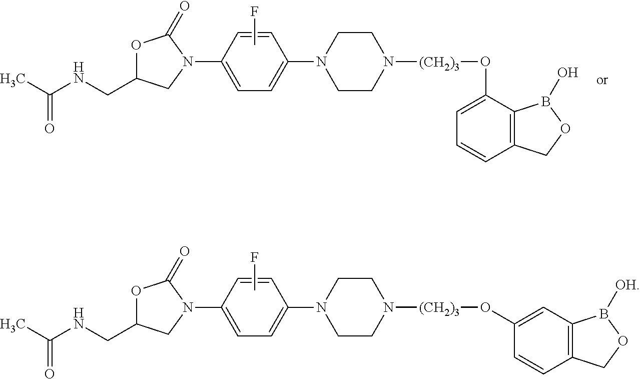

In an exemplary embodiment, the compound has a structure which is

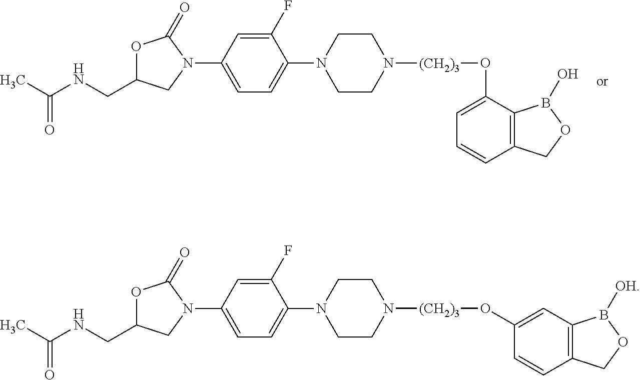

In an exemplary embodiment, the compound has a structure which is

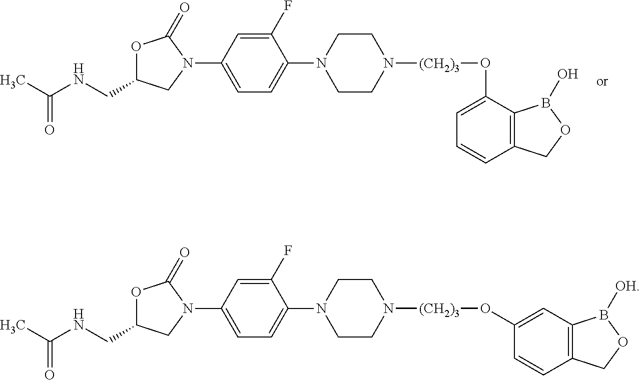

In an exemplary embodiment, the compound has a structure which is selected from the group consisting of

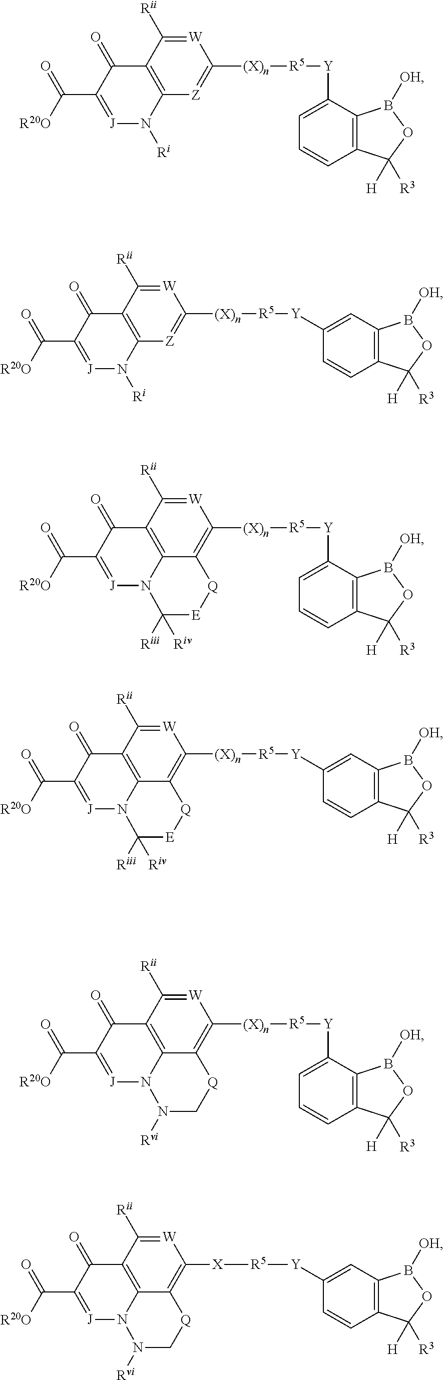

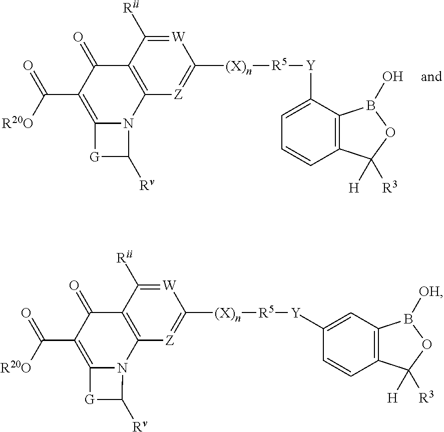

wherein Rⁱ, Rⁱⁱ, Rⁱⁱⁱ, R^{iv}, R^{v}, R²⁰, J, Q, G, E, W, Z, X, R⁵, Y and n are as described herein.

In an exemplary embodiment, the compound has a structure which is selected from the group consisting of

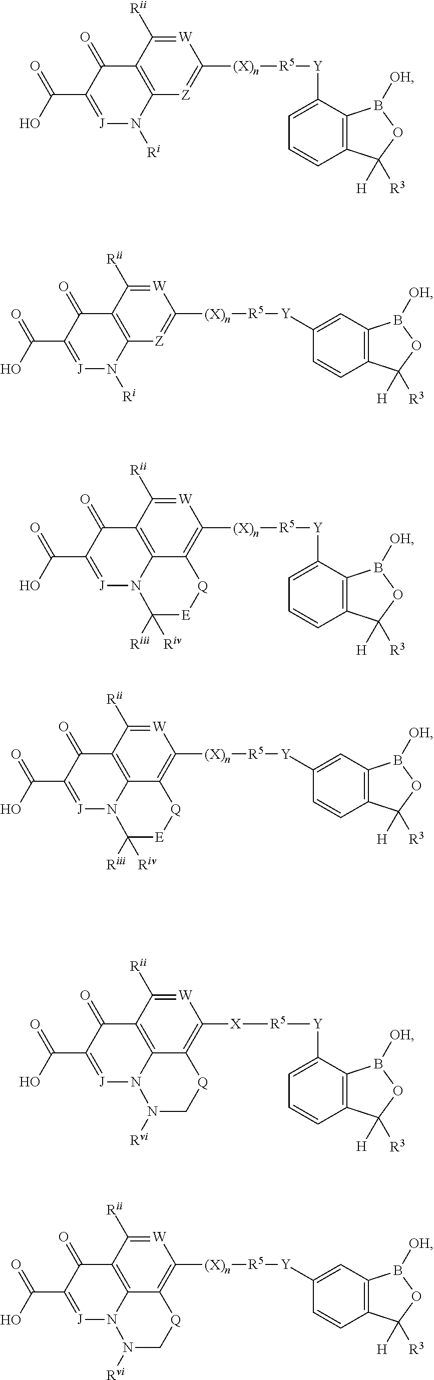

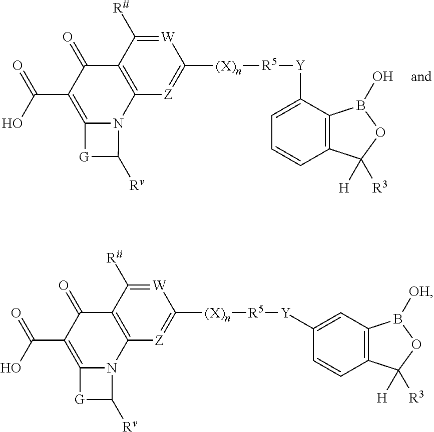

wherein Rⁱ, Rⁱⁱ, Rⁱⁱⁱ, R^{iv}, R^{v}, J, Q, G, E, W, Z, X, R⁵, Y and n are as described herein.

In an exemplary embodiment, the compound has a structure which is selected from the group consisting of

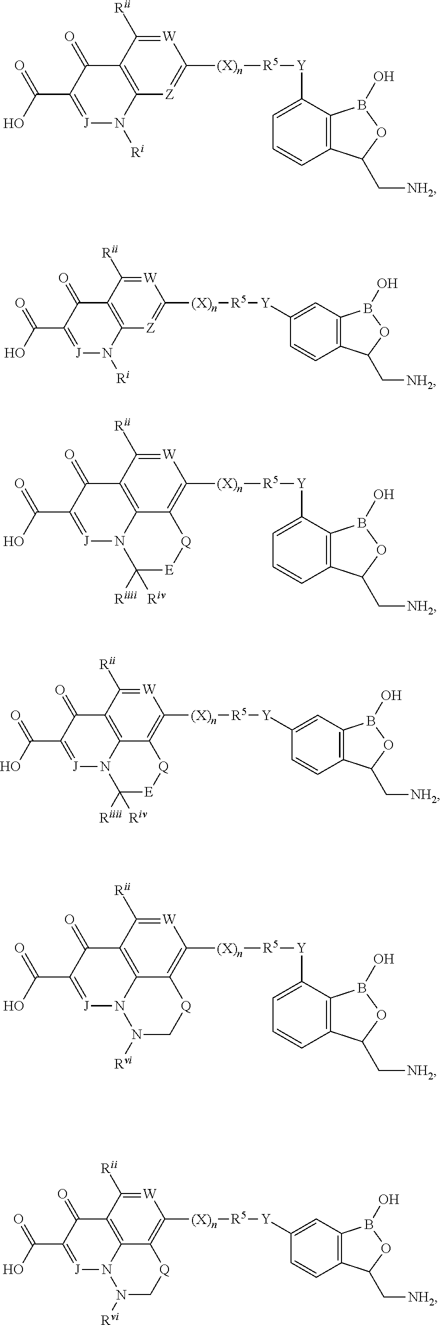

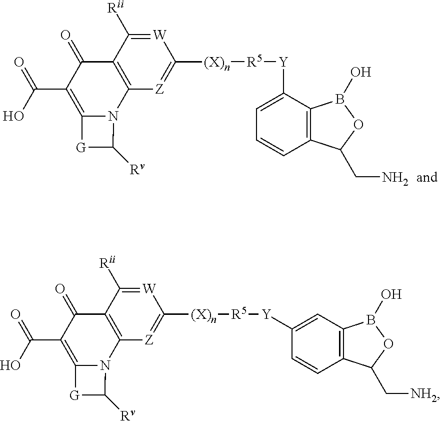

wherein Rⁱ, Rⁱⁱ, Rⁱⁱⁱ, R^{iv}, R^{v}, J, Q, G, E, W, Z, X, R⁵, Y and n are as described herein.

In an exemplary embodiment, the compound has a structure which is selected from the group consisting of

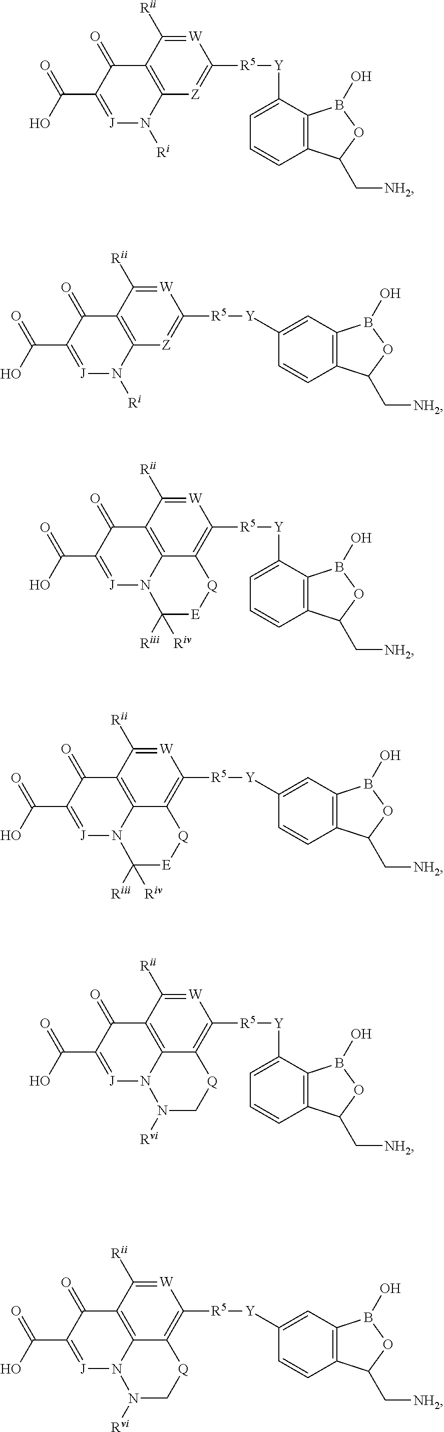

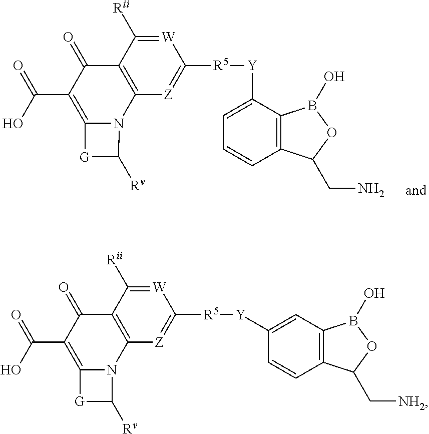

wherein Rⁱ, Rⁱⁱ, Rⁱⁱⁱ, R^{iv}, R^{v}, J, Q, G, E, W, Z, R⁵ and Y are as described herein.

In an exemplary embodiment, the compound has a structure which is selected from the group consisting of

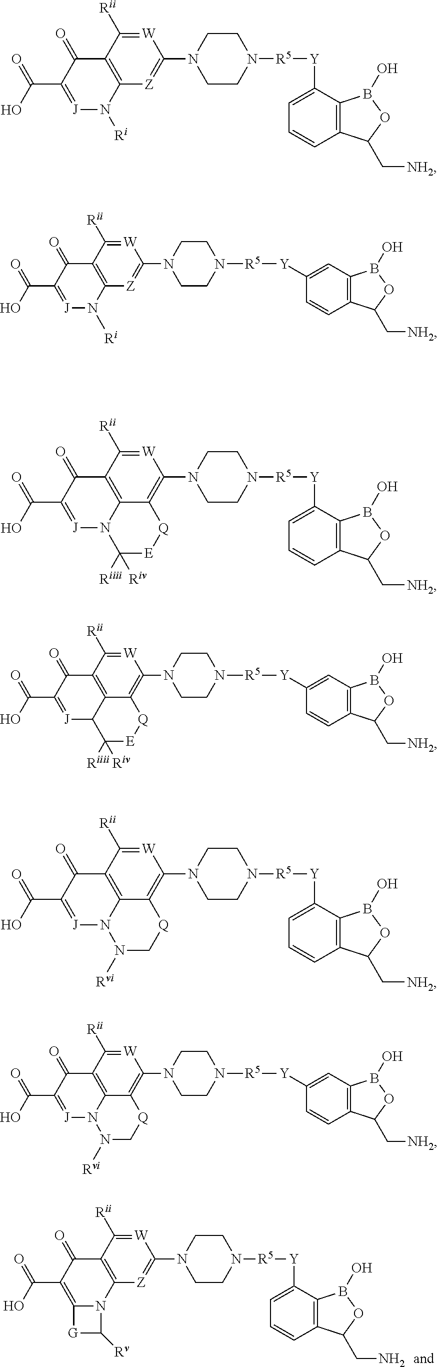

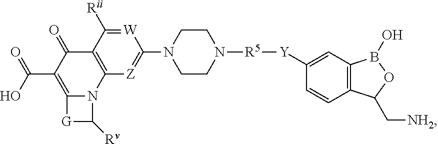

wherein Rⁱ, Rⁱⁱ, Rⁱⁱⁱ, R^{iv}, R^{v}, J, Q, G, E, W, Z, R⁵ and Y are as described herein.

In an exemplary embodiment, the compound has a structure which is selected from the group consisting of

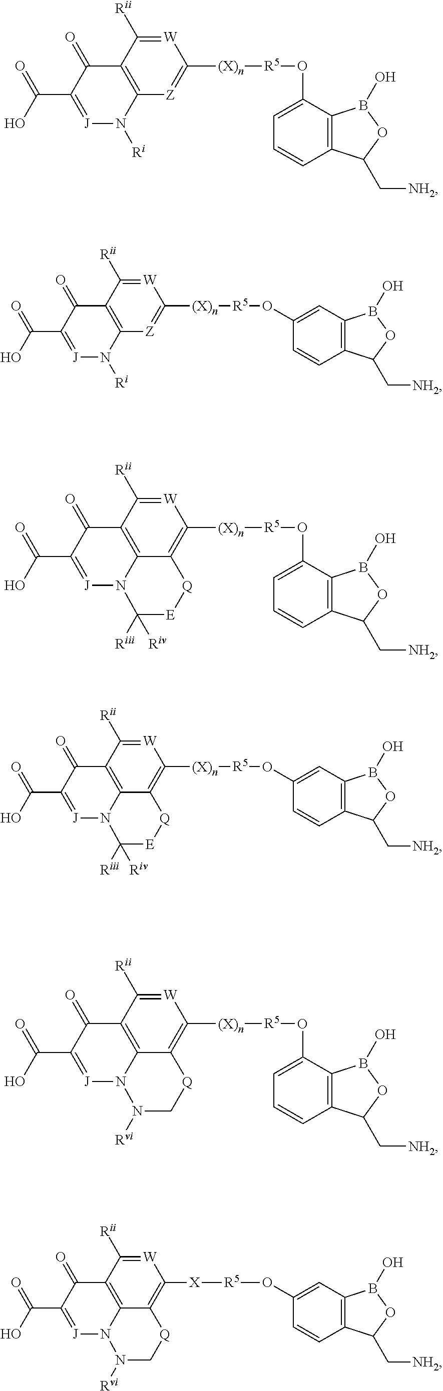

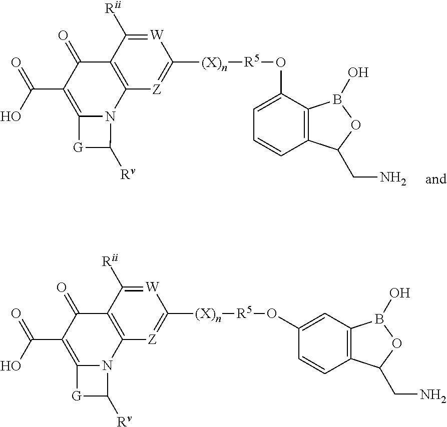

wherein Rⁱ, Rⁱⁱ, Rⁱⁱⁱ, R^{iv}, R^{v}, J, G, E, W, Z, R⁵ and Y are as described herein.

In an exemplary embodiment, the compound has a structure which is selected from the group consisting of

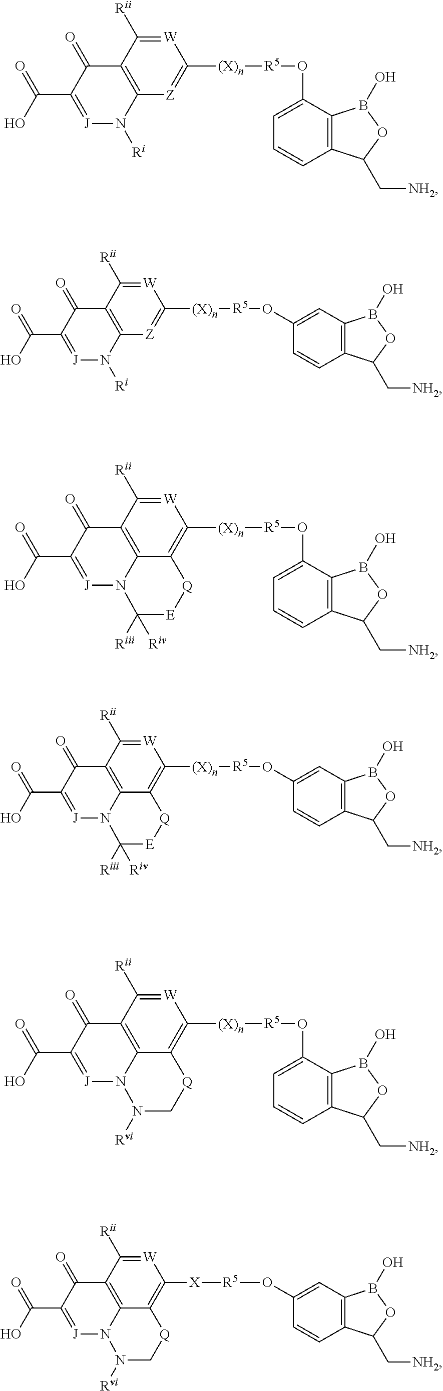

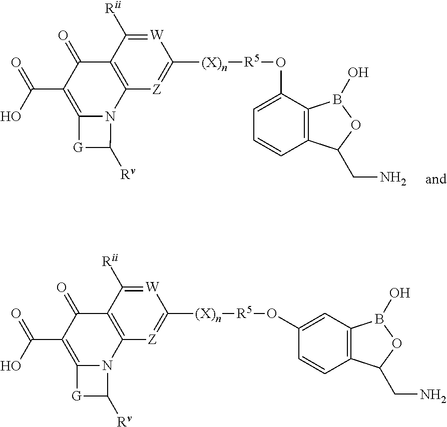

wherein Rⁱ, Rⁱⁱ, Rⁱⁱⁱ, R^{iv}, R^{v}, J, Q, G, E, W, Z, R⁵ and Y are as described herein.

In an exemplary embodiment, the compound has a structure which is selected from the group consisting of

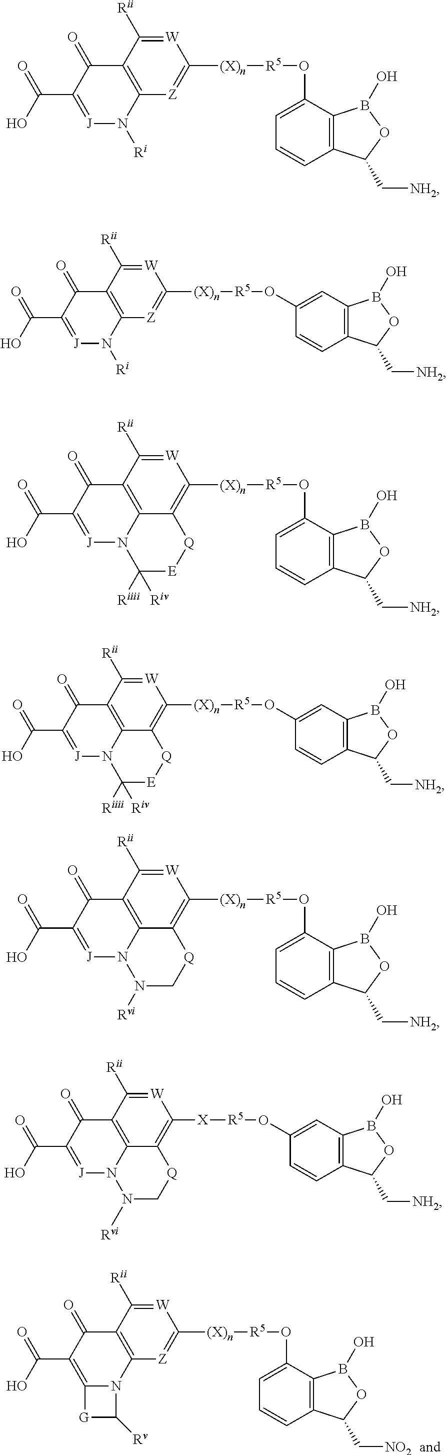

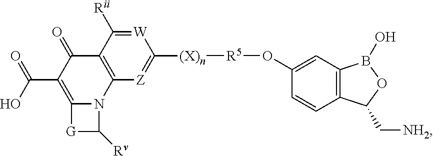

wherein Rⁱ, Rⁱⁱ, Rⁱⁱⁱ, R^{iv}, R^{v}, J, Q, G, E, W, Z, R⁵ and Y are as described herein.

In an exemplary embodiment, the compound has a structure which is selected from the group consisting of

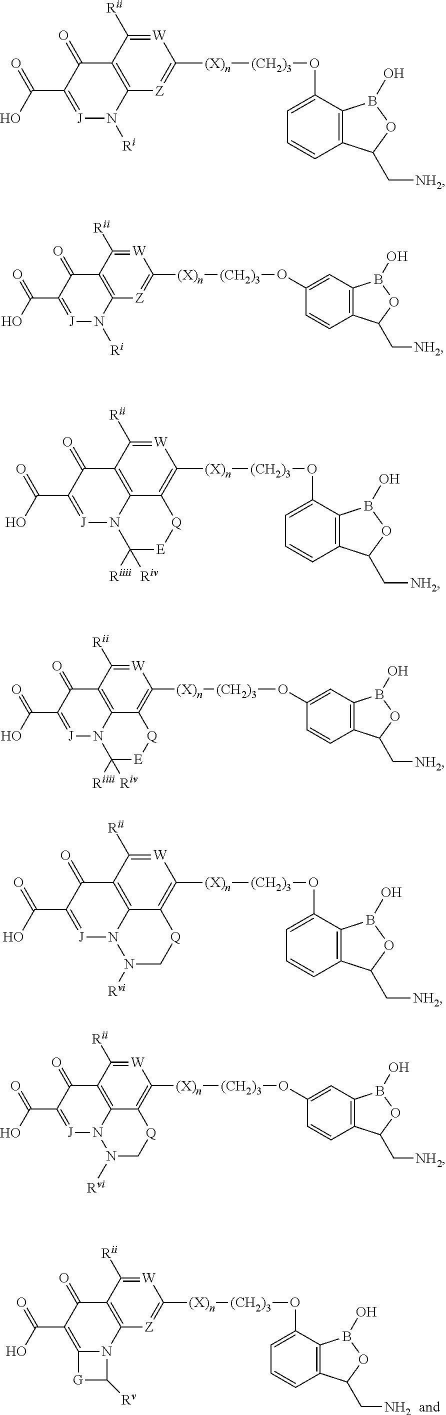

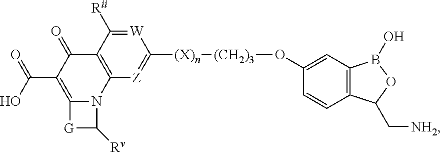

wherein Rⁱ, Rⁱⁱ, Rⁱⁱⁱ, R^{iv}, R^{v}, J, Q, G, E, X, n, W, Z, R⁵ and Y are as described herein.

In an exemplary embodiment, the compound has a structure which is selected from the group consisting of

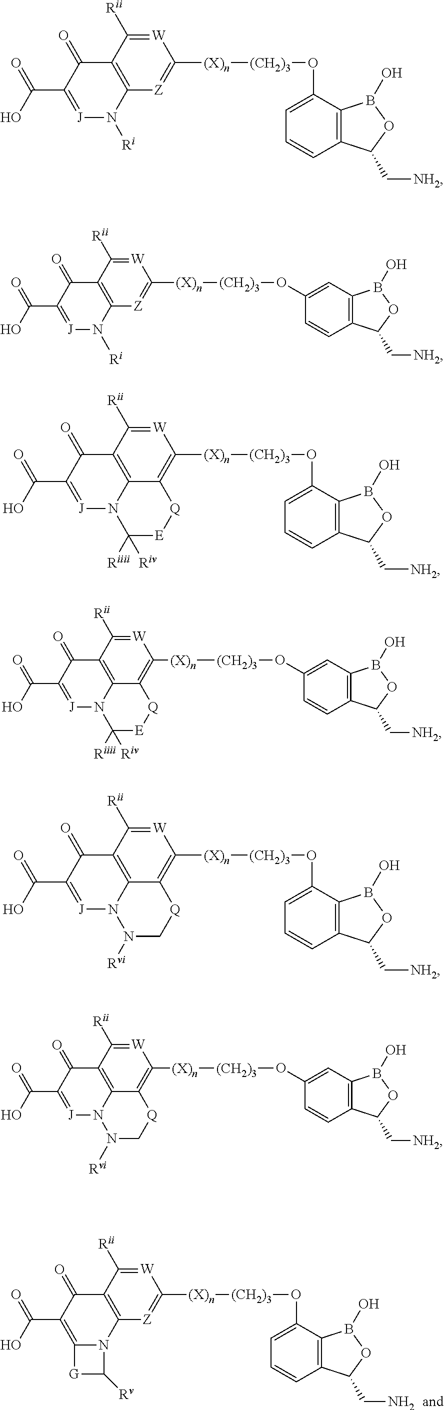

wherein Rⁱ, Rⁱⁱ, Rⁱⁱⁱ, R^{iv}, R^{v}, J, Q, G, E, X, n, W, Z, R⁵ and Y are as described herein.

In an exemplary embodiment, the compound has a structure which is selected from the group consisting of

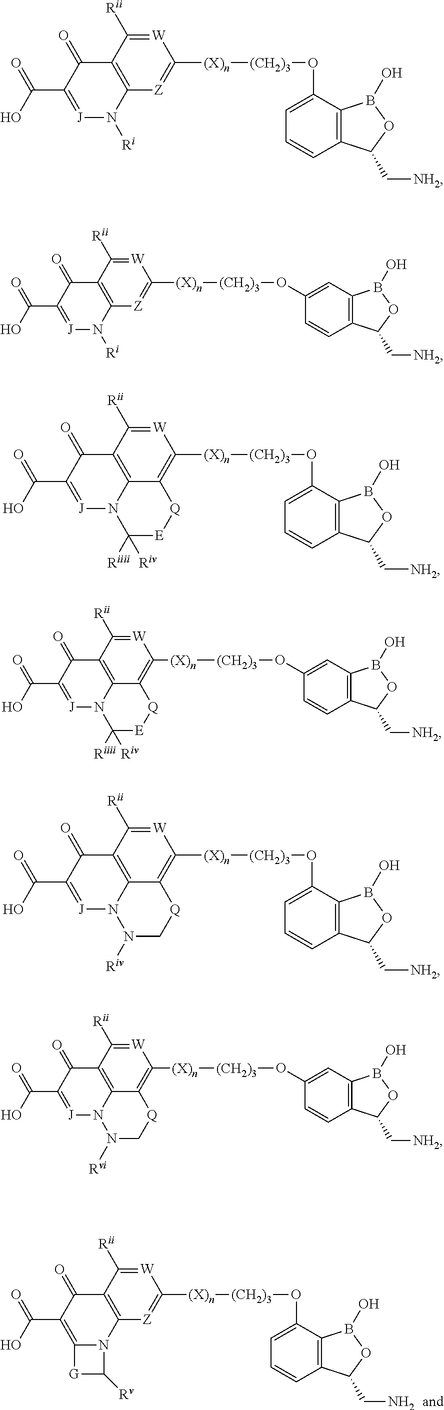

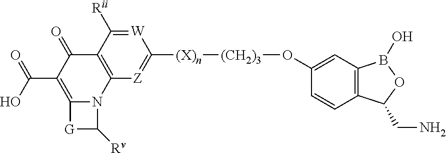

wherein Rⁱ, Rⁱⁱ, Rⁱⁱⁱ, R^{iv}, R^{v}, J, Q, G, E, X, n, W, Z, R⁵ and Y are as described herein.

In an exemplary embodiment, the compound has a structure which is

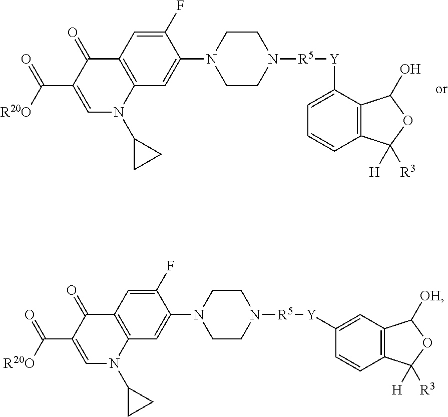

wherein R²⁰, R³, R⁵ and Y are as described herein.

In an exemplary embodiment, the compound has a structure which is

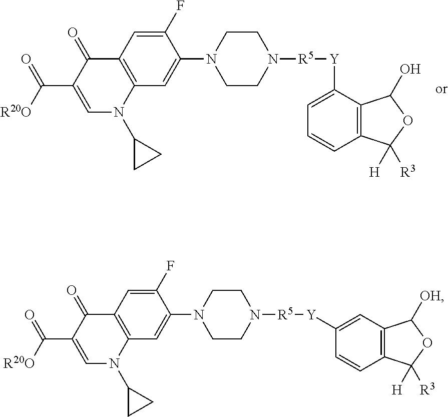

wherein R²⁰, R⁵ and Y are as described herein.

In an exemplary embodiment, the compound has a structure which is

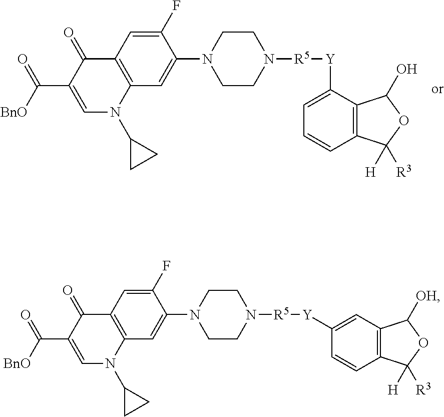

wherein R⁵ and Y are as described herein.

In an exemplary embodiment, the compound has a structure which is

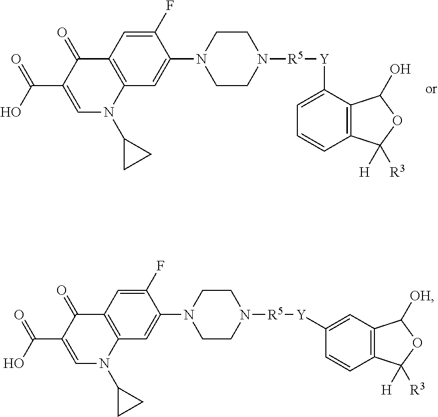

wherein R⁵ and Y are as described herein.

In an exemplary embodiment, the compound has a structure which is

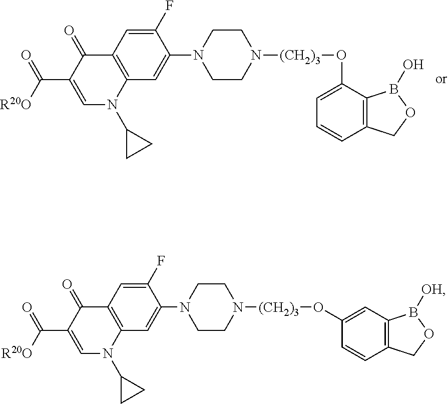

wherein R²⁰ is as described herein.

In an exemplary embodiment, the compound has a structure which is

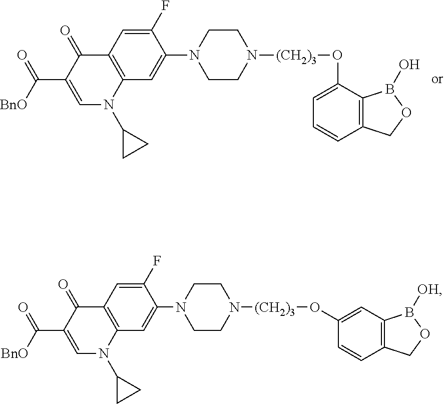

In an exemplary embodiment, the compound has a structure which is

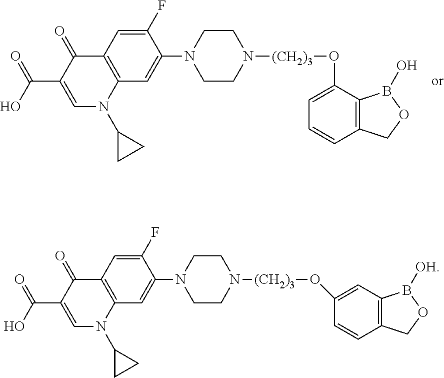

In an exemplary embodiment, the compound has a structure which is

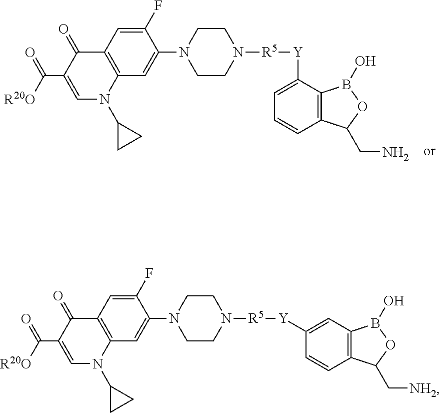

wherein R²⁰, Y and R⁵ is as described herein.

In an exemplary embodiment, the compound has a structure which is

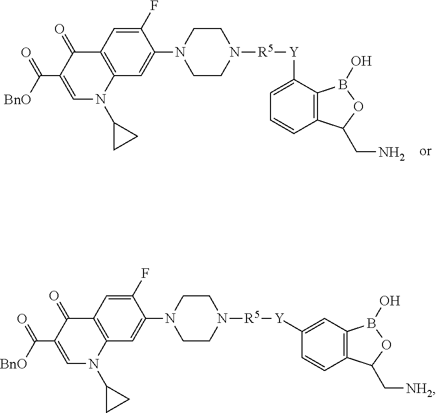

wherein Y and R⁵ is as described herein.

In an exemplary embodiment, the compound has a structure which is

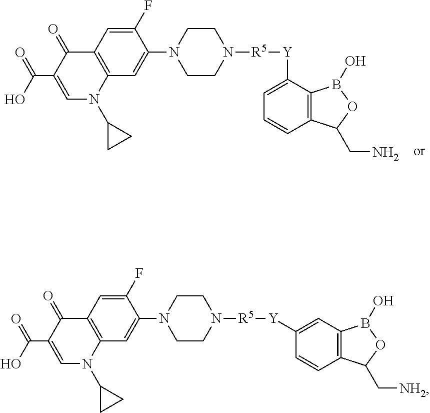

wherein Y and R⁵ is as described herein.

In an exemplary embodiment, the compound has a structure which is

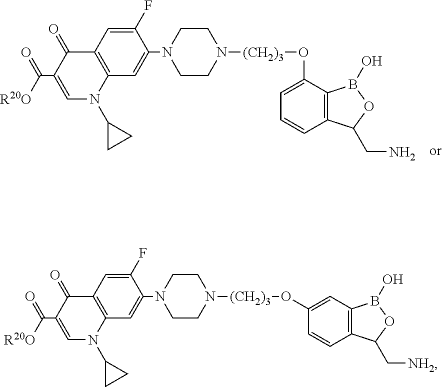

wherein R²⁰ is as described herein.

In an exemplary embodiment, the compound has a structure which is

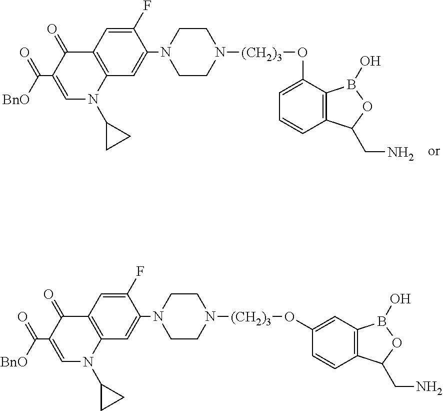

In an exemplary embodiment, the compound has a structure which is

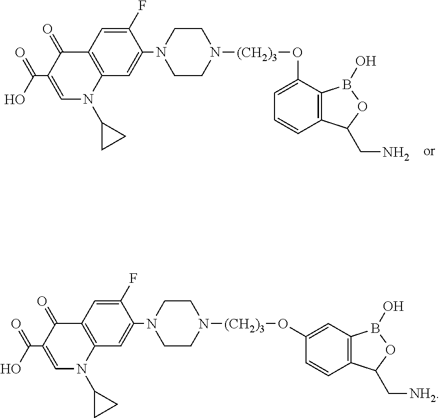

In an exemplary embodiment, the compound has a structure which is

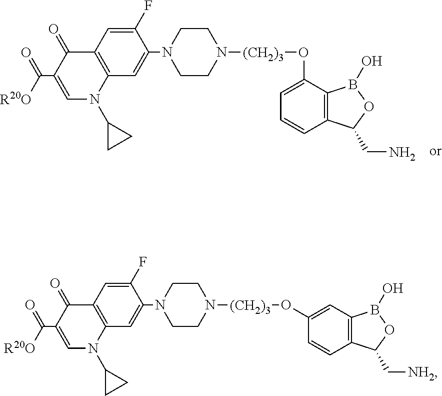

wherein R²⁰ is as described herein.

In an exemplary embodiment, the compound has a structure which is

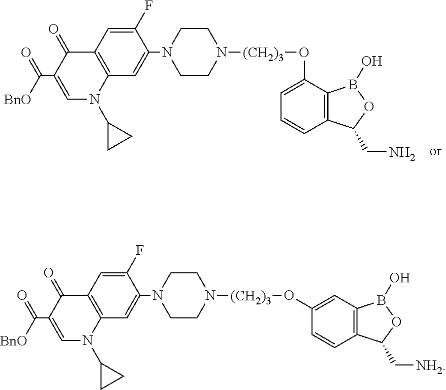

In an exemplary embodiment, the compound has a structure which is

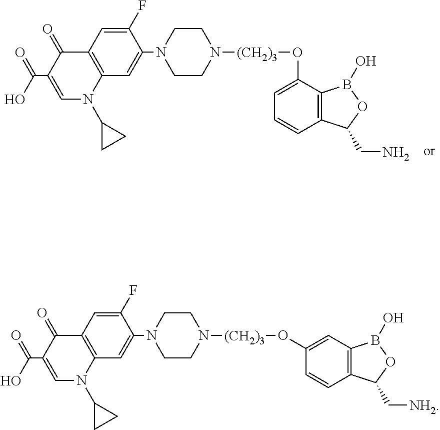

In an exemplary embodiment, the compound has a structure which is

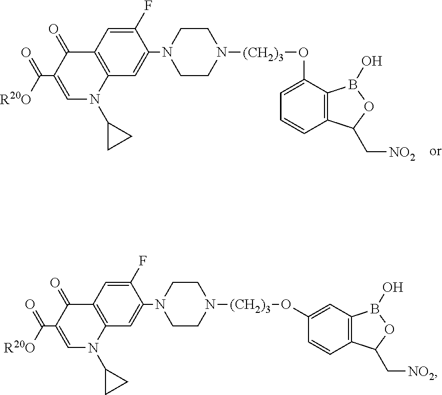

wherein R²⁰ is as described herein.

In an exemplary embodiment, the compound has a structure which is

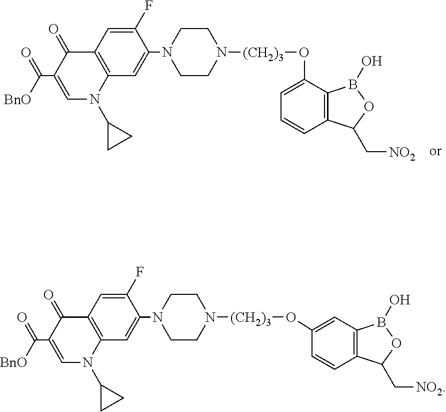

In an exemplary embodiment, the compound has a structure which is

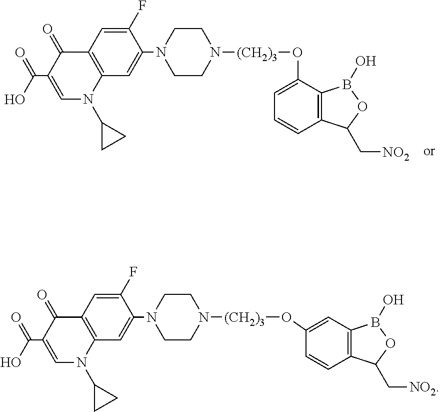

In an exemplary embodiment, the compound has a structure according to the following formulae which is:

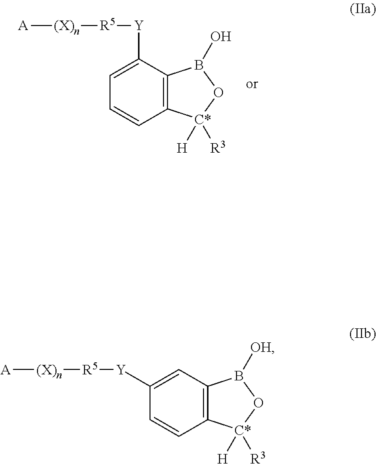

collectively referred to herein as Formula II, wherein A, X, n, R⁵, Y and R³ are as described herein, and C* is a carbon atom, and with the proviso that when R³ is not H, C* is a stereocenter which has a configuration which is (R) or (S).

In an exemplary embodiment, the C* stereocenter is in a configuration which is (R) or (S). In an exemplary embodiment, the C* stereocenter is in a (S) configuration. In an exemplary embodiment, the C* stereocenter is in a (S) configuration and R³ is —CH₂NH₂.

In an exemplary embodiment, the invention provides a compound described herein, or a salt, hydrate or solvate thereof, or a combination thereof. In an exemplary embodiment, the invention provides a compound described herein, or a salt, hydrate or solvate thereof. In an exemplary embodiment, the invention provides a compound described herein, or a salt thereof. In an exemplary embodiment, the salt is a pharmaceutically acceptable salt. In an exemplary embodiment, the invention provides a compound described herein, or a hydrate thereof. In an exemplary embodiment, the invention provides a compound described herein, or a solvate thereof. In an exemplary embodiment, the invention provides a compound described herein, or a prodrug thereof. In an exemplary embodiment, the invention provides a salt of a compound described herein. In an exemplary embodiment, the invention provides a pharmaceutically acceptable salt of a compound described herein. In an exemplary embodiment, the invention provides a hydrate of a compound described herein. In an exemplary embodiment, the invention provides a solvate of a compound described herein. In an exemplary embodiment, the invention provides a prodrug of a compound described herein.

In an exemplary embodiment, alkyl is linear alkyl. In another exemplary embodiment, alkyl is branched alkyl.

In an exemplary embodiment, heteroalkyl is linear heteroalkyl. In another exemplary embodiment, heteroalkyl is branched heteroalkyl.

### III.b) Compositions Involving Stereoisomers

As used herein, the term “chiral”, “enantiomerically enriched” or “diastereomerically enriched” refers to a composition having an enantiomeric excess (ee) or a diastereomeric excess (de) of greater than about 50%, preferably greater than about 70% and more preferably greater than about 90%. In general, higher than about 90% enantiomeric or diastereomeric excess is particularly preferred, e.g., those compositions with greater than about 95%, greater than about 97% and greater than about 99% ee or de.

When a first compound and a second compound are present in a composition, and the first compound is a non-superimposable mirror image of the second compound, and the first compound is present in the composition in a greater amount than the second compound, then the first compound is referred to herein as being present in “enantiomeric excess”.

The term “enantiomeric excess” of a compound z, as used herein, is defined as:

$${ee}_{z} = ( \frac{conc . of z - conc . of y}{conc . of z + conc . of y} ) \times 100$$

wherein z is a first compound in a composition, y is a second compound in the composition, and the first compound is a non-superimposable mirror image of the second compound.

The term “enantiomeric excess” is related to the older term “optical purity” in that both are measures of the same phenomenon. The value of ee will be a number from 0 to 100, zero being racemic and 100 being enantiomerically pure. A composition which in the past might have been called 98% optically pure is now more precisely characterized by 96% ee. A 90% ee reflects the presence of 95% of one enantiomer and 5% of the other(s) in the material in question.

When a first compound and at least one additional compound are present in a composition, and the first compound and each of the additional compounds are stereoisomers, but not mirror images, of one another, and the first compound is present in the composition in a greater amount than each of the additional compounds, then the first compound is referred to herein as being present in “diastereomeric excess”.

When dealing with mixtures of diastereomers, the term “diastereomeric excess” or “de” is defined analogously to enantiomeric excess. Thus:

$${de}_{w} = ( \frac{\begin{matrix}conc . of major diastereomer - \\ conc . of min or diastereomer ( s )\end{matrix}}{\begin{matrix}conc . of major diastereomer + \\ conc . of min or diastereomer ( s )\end{matrix}} ) \times 100$$

wherein the major diastereomer is a first compound in a composition, and the minor diastereomer(s) is at least one additional compound in the composition, and the major diastereomer and minor diastereomer(s) are stereoisomers, but not mirror images, of one another.

The value of de will likewise be a number from 0 to 100, zero being an equal mixture of a first diastereomer and the remaining diastereomer(s), and 100 being 100% of a single diastereomer and zero % of the other(s)—i.e. diastereomerically pure. Thus, 90% de reflects the presence of 95% of one diastereomer and 5% of the other diastereomer(s) in the material in question.

Hence, in one embodiment, the invention provides a composition including a first compound of the invention, wherein the first compound of the invention has at least one stereocenter, and at least one stereoisomer of the first compound of the invention. In another embodiment, the invention provides a composition including a first compound of the invention, wherein the first compound of the invention has at least one stereocenter, and a second compound of the invention, wherein the first compound of the invention is a stereoisomer of the second compound of the invention. In another embodiment, the invention provides a composition including a first compound of the invention, wherein the first compound of the invention has at least one stereocenter, and only one stereoisomer of the first compound of the invention.

In another embodiment, the invention provides a composition including a first compound of the invention, wherein the first compound of the invention has only one stereocenter, and an enantiomer of the first compound of the invention. In another embodiment, the invention provides a composition including a first compound of the invention, wherein the first compound of the invention has two stereocenters, and an enantiomer of the first compound of the invention. In another embodiment, the invention provides a composition including a first compound of the invention, wherein the first compound of the invention has two stereocenters, and at least one diastereomer of the first compound of the invention. In another embodiment, the invention provides a composition including a first compound of the invention, wherein the first compound of the invention has two stereocenters, and only one diastereomer of the first compound of the invention.

In situations where the first compound of the invention and its enantiomer are present in a composition, the first compound of the invention can be present in an enantiomeric excess of at least about 80%, or at least about 90%, or at least about 92% or at least about 95%. In another embodiment, where the first compound of the invention and its enantiomer are present in a composition, the first compound of the invention can be present in an enantiomeric excess of at least about 96%, at least about 97%, at least about 98%, at least about 99% or at least about 99.5%. In another embodiment, the first compound of the invention has at least one stereocenter and is enantiomerically pure (enantiomeric excess is about 100%).

In situations where the first compound of the invention and at least one diastereomer of the first compound of the invention are present in a composition, the first compound of the invention can be present in a diastereomeric excess of at least about 80%, or at least about 90%, or at least about 92% or at least about 95%. In situations where the first compound of the invention and at least one diastereomer of the first compound of the invention are present in a composition, the first compound of the invention can be present in a diastereomeric excess of at least about 96%, at least about 97%, at least about 98%, at least about 99% or at least about 99.5%. In another embodiment, the first compound of the invention has at least two stereocenters and is diastereomerically pure (diastereomeric excess is about 100%).

Enantiomeric or diastereomeric excess can be determined relative to exactly one other stereoisomer, or can be determined relative to the sum of at least two other stereoisomers. In an exemplary embodiment, enantiomeric or diastereomeric excess is determined relative to all other detectable stereoisomers, which are present in the mixture. Stereoisomers are detectable if a concentration of such stereoisomer in the analyzed mixture can be determined using common analytical methods, such as chiral HPLC.

As used herein, and unless otherwise indicated, a composition that is “substantially free” of a compound means that the composition contains less than about 20% by weight, or less than about 15% by weight, or less than about 10% by weight, or less than about 5% by weight, or less than about 3% by weight, or less than about 2% by weight, or less than about 1% by weight of the compound.

As used herein, the term “substantially free of the (or its) enantiomer” means that a composition contains a significantly greater proportion of a first compound of the invention than a second compound of the invention, wherein the first compound is a non-superimposable mirror image of the second compound. In one embodiment of the invention, the term “substantially free of the enantiomer” means that the composition is made up of at least about 90% by weight of a first compound of the invention, and about 10% by weight or less of a second compound of the invention, wherein the first compound is a non-superimposable mirror image of the second compound. In one embodiment of the invention, the term “substantially free of the (R) enantiomer” means that the composition is made up of at least about 90% by weight of a first compound of the invention which has only one stereocenter and the stereocenter is in an (S) configuration, and about 10% by weight or less of a second compound of the invention, wherein the second compound is the enantiomer of the first compound. In one embodiment of the invention, the term “substantially free of the enantiomer” means that the composition is made up of at least about 95% by weight of a first compound of the invention, and about 5% by weight or less of a second compound of the invention, wherein the first compound is a non-superimposable mirror image of the second compound. In one embodiment of the invention, the term “substantially free of the (R) enantiomer” means that the composition is made up of at least about 95% by weight of a first compound of the invention which has only one stereocenter and the stereocenter is in an (S) configuration, and about 5% by weight or less of a second compound of the invention, wherein the second compound is the enantiomer of the first compound. In one embodiment of the invention, the term “substantially free of the enantiomer” means that the composition is made up of at least about 98% by weight of a first compound of the invention, and about 2% by weight or less of a second compound of the invention, wherein the first compound is a non-superimposable mirror image of the second compound. In one embodiment of the invention, the term “substantially free of the (R) enantiomer” means that the composition is made up of at least about 98% by weight of a first compound of the invention which has only one stereocenter and the stereocenter is in an (S) configuration, and about 2% by weight or less of a second compound of the invention, wherein the second compound is the enantiomer of the first compound. In one embodiment of the invention, the term “substantially free of the enantiomer” means that the composition is made up of at least about 99% by weight of a first compound of the invention, and about 1% by weight or less of a second compound of the invention, wherein the first compound is a non-superimposable mirror image of the second compound. In one embodiment of the invention, the term “substantially free of the (R) enantiomer” means that the composition is made up of at least about 99% by weight of a first compound of the invention which has only one stereocenter and the stereocenter is in an (S) configuration, and about 1% by weight or less of a second compound of the invention, wherein the second compound is the enantiomer of the first compound.

In an exemplary embodiment, the invention provides a composition comprising a) first compound described herein; and b) the enantiomer of the first compound, wherein the first compound described herein is present in an enantiomeric excess of at least 80%. In an exemplary embodiment, the enantiomeric excess is at least 92%. In an exemplary embodiment, the first compound described herein has a structure according to Formula II. In another exemplary embodiment, the first compound described herein has a structure according to Formula II, and the C* stereocenter is in a (S) configuration, and the C* stereocenter is the only stereocenter in the first compound. In another exemplary embodiment, the first compound described herein has a structure according to Formula II, and the C* stereocenter is in a (S) configuration, and the C* stereocenter is the only stereocenter in the first compound and R³ is —CH₂NH₂.

In an exemplary embodiment, the invention provides a composition comprising a first compound described herein with a structure according to Formula II, and the C* stereocenter is in a (S) configuration, and said composition is substantially free of the enantiomer of the first compound described herein. In an exemplary embodiment, the invention provides a composition comprising a first compound described herein with a structure according to Formula II, wherein the C* stereocenter is in a (R) configuration, and said composition is substantially free of the enantiomer of the first compound described herein.

### III.c) Combinations Comprising Additional Therapeutic Agents

The compounds of the invention may also be used in combination with additional therapeutic agents. The invention thus provides, in a further aspect, a combination comprising a compound described herein or a pharmaceutically acceptable salt thereof together with at least one additional therapeutic agent. In an exemplary embodiment, the additional therapeutic agent is a compound of the invention. In an exemplary embodiment, the additional therapeutic agent includes a boron atom. In an exemplary embodiment, the additional therapeutic agent does not contain a boron atom. In an exemplary embodiment, the additional therapeutic agent is a compound described in section III.c).

When a compound of the invention is used in combination with a second therapeutic agent active against the same disease state, the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. In an exemplary embodiment, the additional therapeutic agent is an antibiotic. Examples of classes of antibiotics which may be utilized in the application include an aminoglycoside, an ansamycin, a carbacephem, a carbapenem, a first-generation cephalosporin, a second-generation cephalosporin, a third-generation cephalosporin, a fourth-generation cephalosporin, a fifth-generation cephalosporin, a glycopeptide, a macrolide, a quinolone, a sulfonamide, and a tetracycline. In an exemplary embodiment, the additional therapeutic agent is selected from the group consisting of amikacin, gentamicin, kanamycin, neomycin, netilmicin, streptomycin, tobramycin and paromomycin. In an exemplary embodiment, the additional therapeutic agent is geldanamycin or herbimycin. In an exemplary embodiment, the additional therapeutic agent is loracarbef. In an exemplary embodiment, the additional therapeutic agent is selected from the group consisting of ertapenem, doripenem, imipenem, cilastatin and meropenem. In an exemplary embodiment, the additional therapeutic agent is selected from the group consisting of cefadroxil, cefazolin, cefalotin and cefalexin. In an exemplary embodiment, the additional therapeutic agent is selected from the group consisting of cefaclor, cefamandole, cefoxitin, cefprozil and cefuroxime. In an exemplary embodiment, the additional therapeutic agent is selected from the group consisting of cefixime, cefdinir, cefditoren, cefoperazone, cefotaxime, cefpodoxime, ceftazidime, ceftibuten, ceftizoxime and ceftriaxone. In an exemplary embodiment, the additional therapeutic agent is cefepime. In an exemplary embodiment, the additional therapeutic agent is ceftobiprole. In an exemplary embodiment, the additional therapeutic agent is teicoplanin or vancomycin. In an exemplary embodiment, the additional therapeutic agent is selected from the group consisting of azithromycin, clarithromycin, dirithromycin, erythromycin, roxithromycin, troleandomycin, telithromycin and spectinomycin. In an exemplary embodiment, the additional therapeutic agent is aztreonam. In an exemplary embodiment, the additional therapeutic agent is selected from the group consisting of amoxicillin, ampicillin, azlocillin, carbenicillin, cloxacillin, dicloxacillin, flucloxacillin, mezlocillin, meticillin, nafcillin, oxacillin, penicillin, piperacillin and ticarcillin. In an exemplary embodiment, the additional therapeutic agent is selected from the group consisting of bacitracin, colistin and polymyxin B. In an exemplary embodiment, the additional therapeutic agent is selected from the group consisting of ciprofloxacin, enoxacin, gatifloxacin, levofloxacin, lomefloxacin, moxifloxacin, norfloxacin, ofloxacin and trovafloxacin. In an exemplary embodiment, the additional therapeutic agent is selected from the group consisting of mafenide, prontosil, sulfacetamide, sulfamethizole, sulfanilimide, sulfasalazine, sulfisoxazole, trimethoprim and sulfamethoxazole. In an exemplary embodiment, the additional therapeutic agent is selected from the group consisting of demeclocycline, doxycycline, minocycline, oxytetracycline and tetracycline. In an exemplary embodiment, the additional therapeutic agent is selected from the group consisting of arsphenamine, chloramphenicol, lincomycin, ethambutol, fosfomycin, fusidic acid, furazolidone, isoniazid, linezolid, metronidazole, mupirocin, nitrofurantoin, platensimycin, pyrazinamide, quinupristin, rifampin and tinidazole.

The compounds of the invention, or pharmaceutical formulations thereof may also be used in combination with other therapeutic agents, for example immune therapies [e.g. interferon, such as interferon alfa-2a (ROFERON®-A; Hoffmann-La Roche), interferon alpha-2b (INTRON®-A; Schering-Plough), interferon alfacon-1 (INFERGEN®; Intermune), peginterferon alpha-2b (PEGINTRON™; Schering-Plough) or peginterferon alpha-2a (PEGASYS®; Hoffmann-La Roche)], therapeutic vaccines, antifibrotic agents, anti-inflammatory agents [such as corticosteroids or NSAIDs], bronchodilators [such as beta-2 adrenergic agonists and xanthines (e.g. theophylline)], mucolytic agents, anti-muscarinics, anti-leukotrienes, inhibitors of cell adhesion [e.g. ICAM antagonists], anti-oxidants [e.g. N-acetylcysteine], cytokine agonists, cytokine antagonists, lung surfactants and/or antimicrobial. The compositions according to the invention may also be used in combination with gene replacement therapy.

The individual components of such combinations may be administered either simultaneously or sequentially in a unit dosage form. The unit dosage form may be a single or multiple unit dosage forms. In an exemplary embodiment, the invention provides a combination in a single unit dosage form. An example of a single unit dosage form is a capsule wherein both the compound of the invention and the additional therapeutic agent are contained within the same capsule. In an exemplary embodiment, the invention provides a combination in a two unit dosage form. An example of a two unit dosage form is a first capsule which contains the compound of the invention and a second capsule which contains the additional therapeutic agent. Thus the term ‘single unit’ or ‘two unit’ or ‘multiple unit’ refers to the object which the patient ingests, not to the interior components of the object. Appropriate doses of known therapeutic agents will be readily appreciated by those skilled in the art.

The combinations referred to herein may conveniently be presented for use in the form of a pharmaceutical formulation. Thus, an exemplary embodiment of the invention is a pharmaceutical formulation comprising a) a compound of the invention; b) an additional therapeutic agent and c) a pharmaceutically acceptable excipient. In an exemplary embodiment, the pharmaceutical formulation is a unit dosage form. In an exemplary embodiment, the pharmaceutical formulation is a single unit dosage form. In an exemplary embodiment, the pharmaceutical formulation is a two unit dosage form. In an exemplary embodiment, the pharmaceutical formulation is a two unit dosage form comprising a first unit dosage form and a second unit dosage form, wherein the first unit dosage form includes a) a compound of the invention and b) a first pharmaceutically acceptable excipient; and the second unit dosage form includes c) an additional therapeutic agent and d) a second pharmaceutically acceptable excipient.

### III.d) Additional Compounds of the Invention

Additional compounds of the invention include those formed between the 2′,3′ diol of the ribose ring of a nucleic acid, nucleoside or nucleotide, and a compound described herein or according to a formula described herein. In an exemplary embodiment, the compound is a cyclic or acyclic boronic ester such as those described herein. These compounds can be used in an animal to kill or inhibit the growth of a microorganism described herein, as well as to treat the diseases described herein. These compounds can be formed in vitro as well as in vivo. Methods of making these compounds are provided in the Examples section.

In another aspect, the invention provides a compound having a structure according to the following formula:

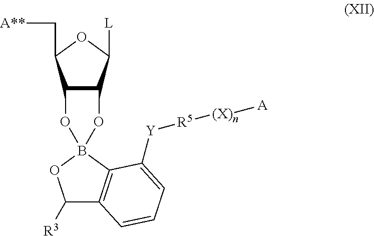

wherein R and R³ are as described herein. L is selected from the group consisting of OR⁷, substituted or unsubstituted purine, substituted or unsubstituted pyrimidine, substituted or unsubstituted pyridine and substituted or unsubstituted imidazole. R⁷ is selected from the group consisting of H, substituted or unsubstituted alkyl, substituted or unsubstituted heteroalkyl, substituted or unsubstituted cycloalkyl, substituted or unsubstituted aryl and substituted or unsubstituted heteroaryl. A** is selected from the group consisting of OH, substituted or unsubstituted monophosphate, substituted or unsubstituted diphosphate, substituted or unsubstituted triphosphate,

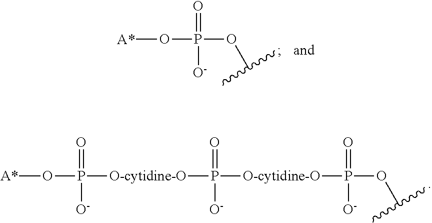

A* is a nucleic acid sequence which comprises between 1 and 100 nucleotides.

In an exemplary embodiment, the compound has the following structure:

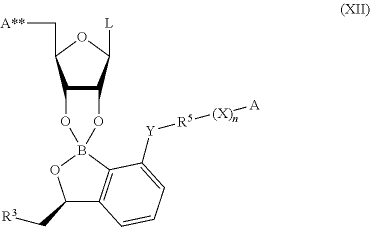

wherein L, R³, Y, R⁵, X, n and A** are as described herein.

### III.e) Preparation of Boron-Containing Editing Domain Inhibitors

Compounds of use in the invention can be prepared using commercially available starting materials or known intermediates. Compounds of use in the invention can be prepared using synthetic methods known in the art or described herein.

The following general procedures were used as indicated in generating the examples and can be applied, using the knowledge of one of skill in the art, to other appropriate compounds to obtain additional analogues.

### General Procedure 1

### Deprotection of Benzyl Protected Alcohols or Thiols

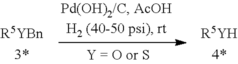

Through subjecting it to deprotection conditions, compound 3* can be converted to compound 4*.

A mixture of the benzylated alcohol or thiol (1 equiv) and 20% Pd(OH)₂ on carbon (50% weight-wet, 1:2 w/w substrate to catalyst) in glacial AcOH (10 mL/g) was shaken under an atmosphere of H₂ (40-50 psi) in a Parr shaker. Once the reaction was complete (TLC), the mixture was filtered through Celite. The filtrate was concentrated in vacuo and the remaining AcOH was removed by co-evaporation with toluene (3×) to give the alcohol. Further purification was carried out by flash chromatography or preparative HPLC as required.

### General Procedure 2

### Phenol or Thiophenol Alkylation Using Mitsunobu Conditions

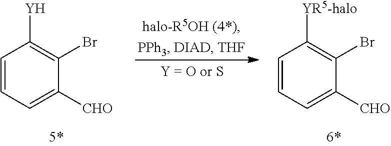

Through subjecting it to Mitsunobu conditions, compound 5* can be converted to compound 6*.

DIAD (1 equiv) was added to a solution of the phenol or thiophenol (1 equiv) and PPh₃ (1 equiv) in anhydrous THF (200 mL/7 g phenol). The mixture was stirred at rt until the reaction was complete (as determined by TLC). The mixture was then concentrated in vacuo. Et₂O was added to the residue and the mixture was then concentrated in vacuo. Et₂O was added again and the precipitate that formed was removed by filtration. The filtrate was extracted with 2 N NaOH and H₂O. The organic layer was dried (Na₂SO₄) and concentrated in vacuo. The residue was further purified by flash chromatography.

### General Procedure 3

### Phenol or Thiophenol Alkylation with Alkyl Bromides and Alkyl Mesylates

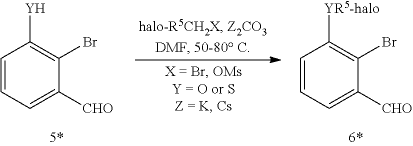

A solution of the alkyl halide or mesylate (1-1.5 equiv), 2-bromo-3-hydroxy-benzaldehyde or 2-bromo-3-mercapto-benzaldehyde (1 equiv), and a base, such as K₂CO₃ (1-1.2 equiv) or Cs₂CO₃ (1.5-2 equiv), in an aprotic solvent such as DMF was stirred at 50-80° C. (bath temp) until the reaction was complete (typically O/N). The reaction mixture cooled to rt, diluted with H₂O, and extracted with a solvent such as EtOAc. The organic fractions were washed with H₂O then brine, dried with a desiccant, such as MgSO₄, and concentrated in vacuo. Further purification was performed by flash chromatography if required.

### General Procedure 4

### Borylation of Aryl Halides and Triflates

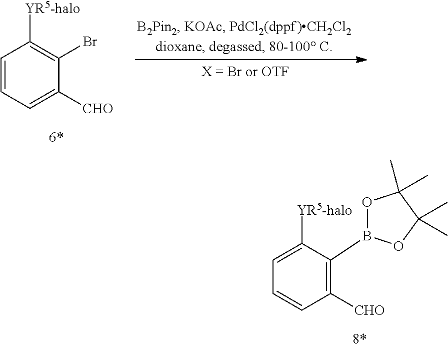

Through subjecting it to borylation conditions, compound 6* can be converted to compound 8*.

A solution of aryl bromide or triflate in anhydrous 1,4-dioxane (20 mL/1 g) was added B₂pin₂ (2 equiv) and KOAc (3 equiv) at rt, then degassed with N₂ for 10 to 40 min. PdCl₂(dppf).CH₂Cl₂ (4-8 mol %) was added and the resulting solution was stirred at 80-100° C. until the reaction was complete (2 to 16 h). The solution was cooled to rt and diluted with EtOAc. The organic layer was then washed with H₂O then brine, dried (Na₂SO₄), filtered, and concentrated in vacuo. The product was typically purified by flash chromatography.

### General Procedure 5

### Borylation of Phenols or Thiophenols Via their Aryl Triflates

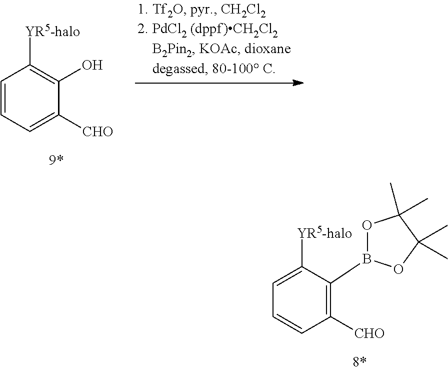

Through subjecting it to borylation conditions, compound 9* can be converted to compound 8*.

Trifluoromethanesulfonic anhydride (1.2 equiv) was added dropwise to a solution of pyridine (1.2 equiv) and the phenol in CH₂Cl₂ (40 mL/8.6 g) at 0° C. (bath temp). The reaction mixture was then allowed to warm to rt and was stirred until complete consumption of starting material (as determined by TLC). Et₂O and 2 N HCl were then added. The organic layer was separated and washed with sat. NaHCO₃ then brine. The organic layer was dried (Na₂SO₄) and filtered through a short silica gel plug, washing with Et₂O. The filtrate was concentrated in vacuo to give the desired triflate that was used directly in general procedure 5.

General procedure 6: Ring Closure of Substituted 2-(4,4,5,5-Tetramethyl-[1,3,2]dioxaborolan-2-O-benzaldehydes

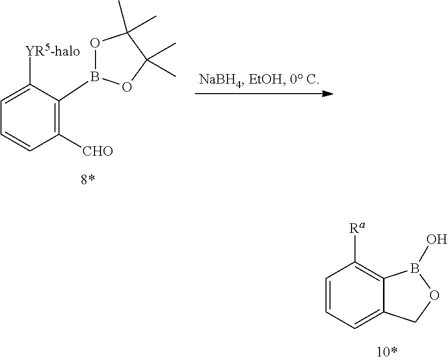

Through subjecting it to ring closure conditions, compound 8* can be converted to compound 10*.

NaBH₄ (1.5 equiv) was added portionwise to an ice-cold solution of the aldehyde in alcohol (typically absolute EtOH or anhydrous MeOH (c=0.1 M). The reaction was allowed to warm to rt and monitored by TLC. The mixture was then acidified to ˜pH 3 using a 1 N NaHSO₄ or 2 M HCl and stirred O/N. The precipitate was collected by filtration, washed repeatedly with H₂O and dried in vacuo. Further purification was carried out by flash chromatography when required.

### General procedure 7: Henry Reaction of Substituted 2-(4,4,5,5-Tetramethyl-[1,3,2]dioxaborolan-2-O-benzaldehydes

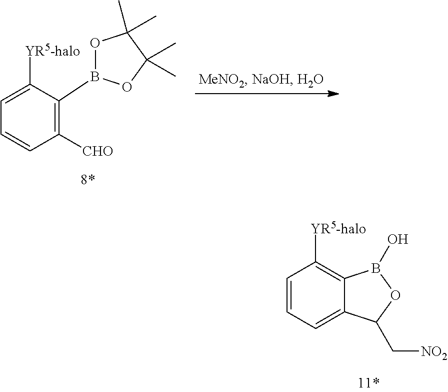

Through subjecting it to Henry reaction conditions, compound 8* can be converted to compound 11*.

NaOH aq. (1.0 equiv) was added to the aldehyde (either in H₂O or THF) at rt and the reaction mixture was stirred at rt for 5 min. MeNO₂ (3 equiv) was added dropwise and the mixture was stirred at rt for 16 h. The reaction mixture was acidified with 2 N HCl and extracted with EtOAc. The organic fraction was washed with H₂O then brine, dried (MgSO₄), and concentrated in vacuo. Purification was typically accomplished by either flash chromatography or precipitation from the acidified reaction mixture.

### General procedure 8: Henry Reaction using Phase Transfer Catalyst of Substituted 2-(4,4,5,5-Tetramethyl-[1,3,2]dioxaborolan-2-yl)-benzaldehydes

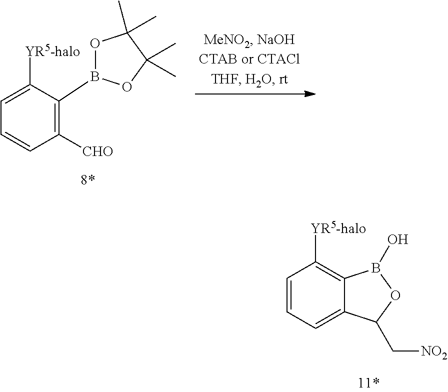

CTAB or CTACl (5 mol %) was added to a mixture of MeNO₂ and aldehyde, in aq. NaOH, and THF (1 mL/300 mg aldehyde) at rt. The reaction was monitored by TLC. Upon completion (typically 1-1.5 h), the mixture was adjusted to pH 2-3 using 2 N HCl or 1 M NaHSO₄ and the mixture was then stirred for 30 min. The solid was filtered and dried to afford the desired nitro compound which was used directly in next step. If there was no precipitation, the organic material was extracted from the reaction mixture with EtOAc. The organic fraction was then dried (MgSO₄) and concentrated in vacuo. The residue was purified by flash chromatography.

### General Procedure 9: N-Alkylation of 7-(3-bromo-propoxy)-3H-benzo[c][1,2]oxaborol-1-ol

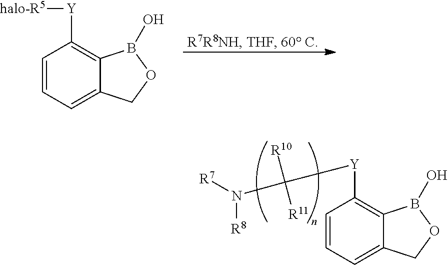

A solution of the 7-(3-bromo-propoxy)-3H-benzo[c][1,2]oxaborol-1-ol (1 equiv.) and di-substituted amine (typically 5 equiv.), in THF was heated at 60° C. (bath temp) in a sealed tube until the reaction was complete (approximately 3 h). The reaction mixture cooled to rt, diluted with H₂O, and extracted with EtOAc. The organic fractions were dried over MgSO₄, filtered and concentrated in vacuo. If required, purification was typically accomplished by preparative HPLC.

### General Procedure 10

### Reduction of Alkyl Nitro and/or Alkyl Nitrile Compounds to N-Boc Protected Amines

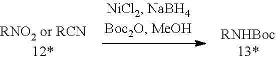

Through subjecting it to reducing conditions, compound 12* can be converted to compound 13*.

Boc₂O (2 equiv) and NiCl₂.6H₂O (1 equiv) were added to a stirred solution of the alkyl nitro or alkyl nitrile in anhydrous MeOH (3 mL/mmol) at rt. Stirring was continued until most of the NiCl₂ had dissolved in MeOH (typically ˜10 min). The reaction mixture was then cooled to 0° C. (bath temp) and NaBH₄ (6 equiv) was added portionwise over 10 min. The reaction was exothermic, effervescent, and resulted in the formation of a finely divided black precipitate. The reaction mixture was allowed to warm to rt and left to stir O/N. The mixture was then concentrated in vacuo and the residue was diluted with EtOAc. The resulting suspension was filtered through Celite and the filtrate was concentrated in vacuo. The residue was then further purified by flash chromatography if required.

### General Procedure 11

### Deprotection of Boc-Protected Amines

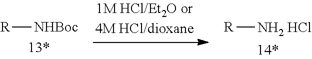

Through subjecting it to deprotection conditions, compound 13* can be converted to compound 14*.

A mixture of the N-Boc protected amine and either 1 M HCl in Et₂O or 4 M HCl in dioxane (2 mL/mmol) was stirred at rt. After the complete consumption of starting material (monitored by TLC, typically 3-16 h), the mixture was concentrated in vacuo and the crude residue was triturated with Et₂O and filtered. If necessary, the final product was purified by preparative HPLC.

### General Procedure 12

### Reduction of Alkyl Nitro and/or Alkyl Nitrile Using Raney Nickel

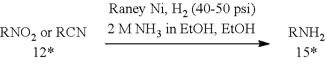

Through subjecting it to reducing conditions, compound 12* can be converted to compound 15*.

A mixture of the 3-nitromethyl-3H-benzo[c][1,2]oxaborol-1-ol, Raney Ni (2 equiv w/w), 2.0 M NH₃ in EtOH (5 mL/1 g), and absolute EtOH (20 mL/1 g) was shaken under an atmosphere of H₂ (40-50 psi) for 3 h at rt. The resultant mixture was filtered through a pad of Celite and washed with EtOH. The filtrate was concentrated in vacuo to give the free amine.

### General procedure 13: Reduction of Substituted-3-nitromethyl-3H-benzo[c][1,2]oxaborol-1-ols using Pearlman's Catalyst

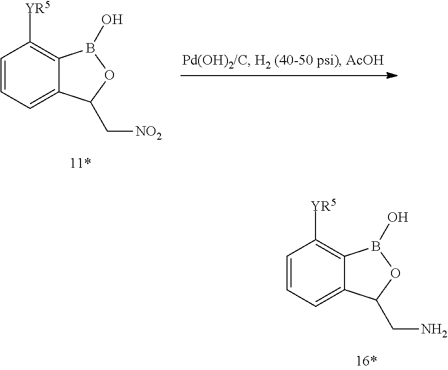

Through subjecting it to reducing conditions, compound 11* can be converted to compound 16*.

A mixture of the 3-nitromethyl-3H-benzo[c][1,2]oxaborol-1-ol (1 equiv) and 20% Pd(OH)₂ on carbon (50% weight-wet, 1:2 w/w substrate to catalyst) in glacial AcOH (10 mL/g) was shaken under an atmosphere of H₂ (45-50 psi) in a Parr shaker. Once the reaction was complete (TLC), the mixture was filtered through Celite. The filtrate was concentrated in vacuo to give a gummy material. The remaining AcOH was removed by co-evaporation with toluene (3×) to give the amine, typically as a fluffy solid. Purification was accomplished by preparative HPLC.

### General Procedure for Chiral HPLC Separation of Enantiomers

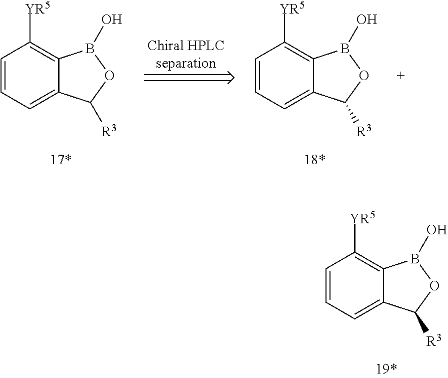

Through subjecting it to chiral HPLC separation conditions, compound 17* can be separated into enantiomers 18* and 19*.

The separation of the two enantiomers was achieved by dissolving the material in a suitable solvent and applying to an appropriate chiral column and eluent system. The collected separated enantiomer samples were then concentrated and used in the next step without further purification. Using this technique, it is possible to achieve a range of enantiomeric excesses of the separated enantiomers.

### General procedure for Chiral Synthesis of 3-aminomethylbenzoxaboroles

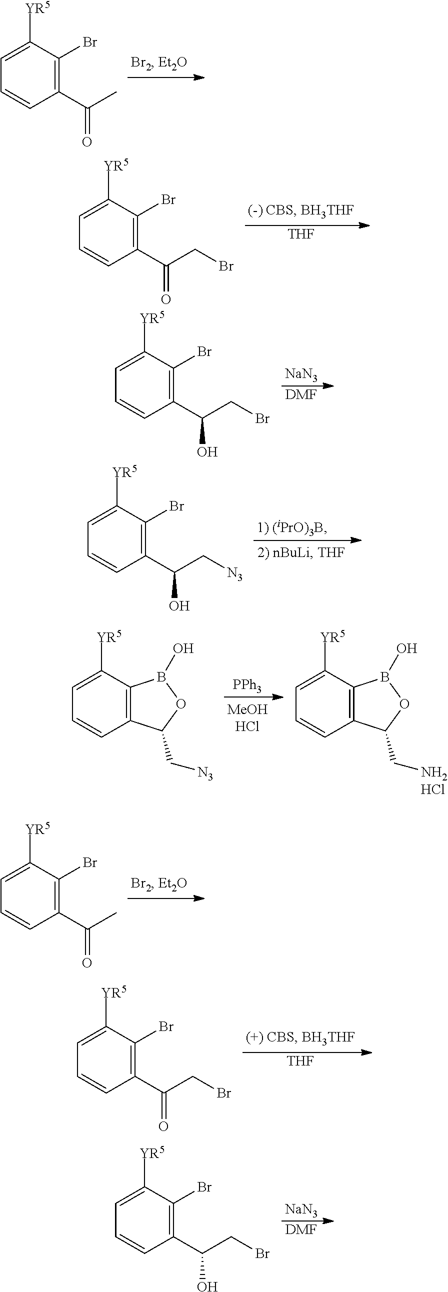

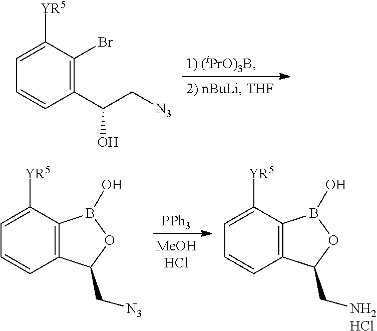

The direct stereospecific synthesis of 3-aminomethylbenzoxaboroles can be achieved starting from the 5- or 6-substituted 2-bromoacetophenone. Bromine (1.0 eq) is added slowly to appropriately substituted 2′-bromoacetophenone (1.0 eq) in diethyl ether at room temperature and stirred for 2 hours. Water is added and the reaction mixture stirred until the color fades. The phases are separated and the aqueous layer extracted with diethyl ether. The combined organic phases are washed with brine, dried over MgSO₄, filtered and concentrated under reduced pressure to give substituted 2-bromo-1-(2-bromophenyl)ethanone. (R)-(+)-2-Methyl-CBS-oxazaborolidine [For R-isomer] or (S)-(−)-2-Methyl-CBS-oxazaborolidine [For S-isomer] (0.11 eq) is added to a stirred solution of substituted 2-bromo-1-(2-bromophenyl)ethanone (1.0 eq) in THF. The reaction mixture is cooled to −10° C. where BH₃.THF (1.0 M in THF, 1.20 eq) is added over 4 hours. The reaction mixture is stirred for a further 45 minutes at −10° C. before the addition of methanol (130 mL). The reaction mixture is concentrated under reduced pressure. The resultant residue is subjected to flash column chromatography to provide the substituted chiral 2-bromo-1-(2-bromophenyl)ethanol. To a solution of this alcohol (1.00 eq) in DMF is added sodium azide at room temperature. The reaction mixture is then heated to 80° C. for 24 hours. Water (150 mL) is added and this solution is extracted with diethyl ether. The combined organic phases are washed with brine (50 mL), dried over MgSO₄, filtered and concentrated under reduced pressure. The residue is subjected to flash column chromatography to yield the substituted 2-azido-1-(2-bromophenyl)ethanol. To a solution of this material (1.00 eq) in toluene is added triisopropyl borate (1.50 eq). The reaction flask is equipped with a Dean and Stark condenser attached and the reaction mixture is refluxed to remove approximately ¾ of the volume of solvent. The dark reaction mixture is cooled to room temperature where THF is added and then cooled to −78° C. n-Butyl lithium (2.5 M in hexanes, 1.15 eq) is added dropwise to the reaction mixture at −78° C. and then stirred for 30 minutes at this temperature. The reaction mixture is then allowed to warm to room temperature where it is stirred for 3 hours before being quenched with 6 M HCl (30 mL). The reaction mixture is concentrated under reduced pressure and the resulting residue is subjected to flash column chromatography to give the substituted 3-(azidomethyl)benzo[c][1,2]oxaborol-1(3H)-ol.

To a solution of this compound (1.0 eq) in methanol is added triphenylphosphine (1.0 eq) and this is stirred for 3 hours at room temperature. Concentrated HCl is added and the reaction mixture stirred for a further 2 hours before being concentrated to dryness under reduced pressure. Dichloromethane is added and extracted with 2 M HCl. The combined aqueous layers are washed with dichloromethane before being contracted under reduced pressure. The residue is then recrystallized from hot water/acetonitrile (3 mL water/50-80 mL acetonitrile per gram of compound) to give the substituted chiral (R or S) 3-(aminomethyl)benzo[c][1,2]oxaborol-1(3H)-ol as the hydrochloride salt.

### General Synthesis of (S)-3-(aminomethyl)-7-(substituted-propoxy)benzo[e][1,2]oxaborol-1(3H)-ols

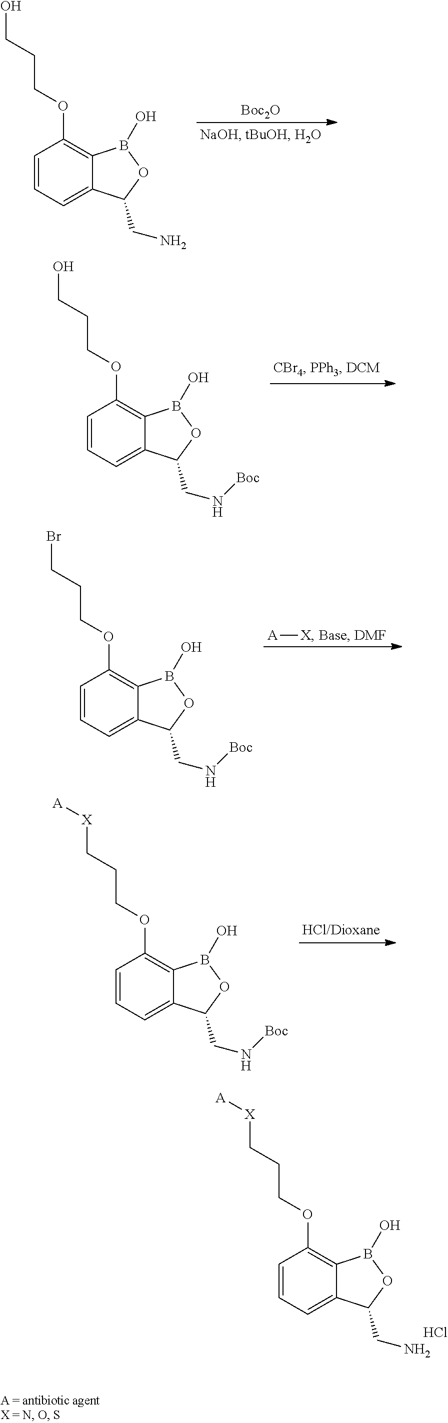

(S)-3-(aminomethyl)-7-(3-hydroxypropoxy)benzo[c][1,2]oxaborol-1(3H)-ol or its HCl salt is dissolved in tBuOH/H₂O (3:1, 20 mL/g) and treated with 1.5 eq of Boc anhydride to form the corresponding Boc-protected derivative. The hydroxyl group is converted to the bromide by combining with 1.1 eq of triphenyl phosphine and dichloromethane (10 mL/g) and cooling to 0° C. before adding 1.1 eq of carbon tetrabromide in dichloromethane (1 mL/g). The reaction mixture is allowed to warm to room temperature and stirred for 18 hrs, after which it is concentrated and suspended in hexane and filtered free of insoluble material and evaporated to obtain the product. The bromide is combined with an antibiotic agent (typically 1.5 eq) containing a reactive nucleophile and treated with an appropriate base (typically 1.5 eq) such as triethyl amine or sodium hydride to form the Boc-protected hybrid antibacterial. Treatment of this material with an acid such as HCl/dioxane removes the Boc protecting group and precipitates the title compound as the HCl salt.

### Methods of Attaching an Antibiotic, or a Pharmacophore of an Antibiotic, to the Boron-Containing Compound

There are many options available for the conjugation of A with X, and/or X with R⁵, and/or A with the boron-containing compound through R⁵. In an exemplary embodiment, the boron-containing compound comprises a reactive functional group, and is conjugated to A, or a precursor of A. In another exemplary embodiment, the boron-containing compound is activated, and then conjugated to A, or a precursor of A. In an exemplary embodiment, A, or a precursor of A, comprises a reactive functional group, and is conjugated to the boron-containing compound. In another exemplary embodiment, A, or a precursor of A is activated, and then conjugated to the boron-containing compound.

The methods of attaching are dependent upon the reactive functional groups present at the site of activation/conjugation. In an exemplary embodiment, the reactive functional group of A, or a precursor of A and the reactive functional group of a boron-containing compound comprise electrophiles and nucleophiles that can generate a covalent linkage between them. Alternatively, the reactive functional group comprises a photoactivatable group, which becomes chemically reactive only after illumination with light of an appropriate wavelength. In an exemplary embodiment, the conjugation reaction between A, or a precursor of A, and the boron-containing compound results in one or more atoms of A or the boron-containing compound being incorporated into a new linkage attaching A to the boron-containing compound. Selected examples of functional groups and linkages are shown in Table 1, where the reaction of an electrophilic group and a nucleophilic group yields a covalent linkage.

TABLE 1
EXAMPLES OF SOME ROUTES TO USEFUL
COVALENT LINKAGES WITH ELECTROPHILE
AND NUCLEOPHILE REACTIVE GROUPS

| Electrophilic Group | Nucleophilic Group | Resulting Covalent Linkage |
|---|---|---|
| activated esters* | amines/anilines | carboxamides |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carboxylic acids | amines/anilines | carboxamides |
| carboxylic acids | alcohols | esters |
| carboxylic acids | hydrazines | hydrazides |
| carbodiimides | carboxylic acids | N-acylureas or anhydrides |
| diazoalkanes | carboxylic acids | esters |
| epoxides | thiols | thioethers |
| haloacetamides | thiols | thioethers |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | ahenols/alcohols | sulfonate esters |

*Activated esters, as understood in the art, generally have the formula —COΩ, where Ω is a good leaving group (e.g. oxysuccinimidyl (—ONC₄H₄O₂) oxysulfosuccinimidyl (—ONC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl).
**Acyl azides can also rearrange to isocyanates

A, or a precursor of A is typically combined with the boron-containing compounds using methods and under conditions (concentration, stoichiometry, pH, temperature and other factors that affect chemical reactions) that are determined by both the reactive groups on the compound and the expected site of modification on the component to be modified. These methods and conditions are generally well known in the art and have been described in detail in Hermanson Greg T., Bioconjugate Techniques, Academic Press, Inc., 1996.

Compounds described herein can be converted into hydrates and solvates by methods similar to those described herein.

### IV. Assays

IVa. Assays for Inhibitors of Ribosomal Initiation Complex Formation

Art-recognized techniques of genetics and molecular biology are of use to identify compounds that bind to and/or inhibit the formation of the ribosomal initiation complex. See, for example, Swaney et al., Antimicrobial Agents and Chemotherapy, (1998), 42(12), 3251-3255.

IVb. Assays for Inhibitors of DNA Gyrase

Art-recognized techniques of genetics and molecular biology are of use to identify compounds that bind to and/or inhibit DNA gyrase. See, for example, Domagala et al., Med. Chem., (1986), 29(3), 394-404.

IVc. Assays for Inhibitors of tRNA Synthetase Editing Domains

Art-recognized techniques of genetics and molecular biology are of use to identify compounds that bind to and/or inhibit the editing domain of a tRNA synthetase. Moreover, these techniques are of use to distinguish whether a compound binds to and/or inhibits the synthetic domain, the editing domain, or both the editing and synthetic domains.

In an exemplary assay, activity of a representative compound against the editing domain was confirmed. To identify the target of a novel boron-containing antibacterial compound, mutants in E. coli showing resistance to the compound were isolated. Characterization of mutants showed that they have an 32-256 fold increase in resistance to the compound over wildtype. The mutants were furthermore shown to be sensitive to various antibacterial agents with known modes of action, suggesting that the cellular target of the compound is distinct from the target of the other antibacterial agents. The leuS gene from the mutants was cloned onto a plasmid and their resistance was confirmed by MIC. The editing domain from these mutants were sequenced and the mutations were all located in the editing domain of this enzyme.

Assays to determine whether, and how effectively, a particular compound binds to and/or inhibits the editing domain of a selected tRNA synthetase are also set forth herein, and additional assays are readily available to those of skill in the art. Briefly, in an exemplary assay, an improperly charged tRNA and a tRNA synthetase that is capable of editing the improperly charged tRNA are combined. The resulting mixture is contacted with the putative inhibitor and the degree of editing inhibition is observed.

Another assay uses genetics to show that a drug works via the editing domain. In this assay, the compound is first tested against a strain of cells over-expressing copies of the tRNA synthetase gene. The compound's effect on the over-expressing strain is compared with a control strain to determine whether the compound is active against the synthetase. If the minimum inhibitory concentration (MIC) is 2-fold higher in the strain with extra copies of the synthetase gene than the MIC of the inhibitor against a wild type cell, a further genetic screen is conducted to determine whether the increased resistance is due to mutations in the editing domain. In this second screen, the control strain is challenged against a high concentration of the inhibitor. The colonies surviving the challenge are isolated and DNA from these cells is isolated. The editing domain is amplified using a proof-reading PCR enzyme and the appropriate primers. The PCR product can be purified using standard procedures. The sequence amplified mutant DNA is compared to wild-type. If the mutant DNA bears mutations in the editing domain, such results would suggest that the compound binds to the editing domain and affects the editing function of the molecule through this domain.

Generally, the compounds to be tested are present in the assays in ranges from about 1 pM to about 100 mM, preferably from about 1 pM to about 1 μM. Other compounds range from about 1 nM to about 100 nM, preferably from about 1 nM to about 1 μM.

The effects of the test compounds upon the function of the enzymes can also be measured by any suitable physiological change. When the functional consequences are determined using intact cells or animals, one can also measure a variety of effects such as transmitter release, hormone release, transcriptional changes to both known and uncharacterized genetic markers, changes in cell metabolism such as cell growth or pH changes, and changes in intracellular second messengers such as Ca²⁺, or cyclic nucleotides.

High throughput screening (HTS) is also of use in identifying promising candidates of the invention.

Utilizing the assays set forth herein and others readily available in the art, those of skill in the art will be able to readily and routinely determine other compounds and classes of compounds that operate to bind to and/or inhibit the editing domain of tRNA synthetases.

In another aspect, the invention provides a method for identifying a compound which binds to an editing domain of a tRNA synthetase comprising: a) contacting said editing domain with a test compound under conditions suitable for binding; and b) detecting binding of said test compound to said editing domain. In an exemplary embodiment, detecting binding of said compound comprises use of at least one detectable element, isotope, or chemical label attached to said compound. In an exemplary embodiment, the element, isotope or chemical label is detected by a fluorescent, luminescent, radioactive, or absorbance readout. In an exemplary embodiment, the contacting of said test compound with said editing domain also includes further contacting said test compound and said editing domain with AMP or a molecule with a terminal adenosine. In an exemplary embodiment, said tRNA synthetase is selected from the group consisting of alanyl tRNA synthetase, isoleucyl tRNA synthetase, leucyl tRNA synthetase, methionyl tRNA synthetase, lysyl tRNA synthetase, phenylalanyl tRNA synthetase, prolyl tRNA synthetase, threonyl tRNA synthetase and valyl tRNA synthetase. In an exemplary embodiment, the tRNA synthetase is derived from leucyl tRNA synthetase. In an exemplary embodiment, the tRNA synthetase is derived from a mutated tRNA synthetase, wherein said mutated tRNA synthetase comprises amino acid mutations in an editing domain. In another exemplary embodiment, wherein said editing domain of a tRNA synthetase comprises the amino acid sequence of a peptide sequence described herein.

In another aspect, the invention provides a method for identifying a compound which binds to an editing domain of a tRNA synthetase, said assay comprising: a) contacting said editing domain of a tRNA synthetase with said compound under conditions suitable for binding of said compound with said editing domain of a tRNA synthetase; b) comparing a biological activity of said editing domain of a tRNA synthetase contacting said compound to said biological activity when not contacting said compound; and c) identifying said compound as binding to said editing domain of a tRNA synthetase if said biological activity of said editing domain of a tRNA synthetase is reduced when contacting said compound. In an exemplary embodiment, the biological activity is hydrolysis of noncognate amino acid. In another exemplary embodiment, the hydrolysis of said noncognate amino acid is detected through the use of one or more labels. In another exemplary embodiment, the labels include a radiolabel, a fluorescent marker, an antibody, or a combination thereof. In another exemplary embodiment, said labels can be detected using spectroscopy. In another exemplary embodiment, the editing domain of a tRNA synthetase is selected from the group consisting of alanyl tRNA synthetase, isoleucyl tRNA synthetase, leucyl tRNA synthetase, methionyl tRNA synthetase, lysyl tRNA synthetase, phenylalanyl tRNA synthetase, prolyl tRNA synthetase, threonyl tRNA synthetase and valyl tRNA synthetase. In another exemplary embodiment, said editing domain of a tRNA synthetase is derived from leucyl tRNA synthetase.

In another aspect, the invention provides a method of generating tRNA molecules with noncognate amino acid comprising: a) creating or isolating a mutated tRNA synthetase with altered amino acid editing domains; and b) contacting a tRNA molecule with said mutated tRNA synthetase and a noncognate amino acid. In another exemplary embodiment, the mutated tRNA synthetase contains one or more amino acid mutations in an editing domain. In another exemplary embodiment, the mutated tRNA synthetase is unable to bind with a compound of the invention. In another exemplary embodiment, the mutated tRNA synthetase is unable to bind with a compound described herein, or a pharmaceutically acceptable salt thereof. In another exemplary embodiment, the mutated tRNA synthetase is unable to bind with a compound according to a formula described herein, or a pharmaceutically acceptable salt thereof. In another exemplary embodiment, R* is H.

In another aspect, the invention provides a composition that comprises one or more tRNA molecules attached to noncognate amino acids, wherein said tRNA molecules are synthesized using one or more mutated tRNA synthetases isolated from a microorganism or a cell line derived from a microorganism. In an exemplary embodiment, the microorganism is a bacteria. In an exemplary embodiment, wherein said mutated tRNA synthetases contain amino acid mutations in their editing domains.

### V. Amino Acid and Nucleotide Sequences Used in Assays

trNA Sequences that Interact with the tRNA Synthetase-Compound of the Invention-AMP Complex

Transfer RNAs (tRNAs) translate mRNA into a protein on a ribosome. Each transfer RNA contains an anti-codon region that hybridizes with mRNA, and an amino acid which may be attached to the growing peptide. The structural gene of tRNA is about 72 to 90 nucleotides long and folds into a cloverleaf structure (Sharp S. J., Schaack J., Coolen L., Burke D. J. and Soll D., “Structure and transcription of eukaryotic tRNA genes”, Crit. Rev. Biochem, 19:107 144 (1985); Geiduschek E. O., and Tocchini-Valentini, “Transcription by RNA polymerase III”, Annu. Rev. Biochem. 57:873 914 (1988)).

In one embodiment, a compound described herein contacts AMP and a tRNA synthetase, and the tRNA synthetase in turn contacts a tRNA molecule. In another embodiment, a compound described herein contacts AMP from the tRNA molecules and a tRNA synthetase. The nucleotide sequence of the tRNA molecule can be determined by the identity of the tRNA synthetase involved. For example, for leucyl tRNA synthetase, the cognate tRNA molecule bound will be tRNA-leucine (SEQ ID NO: 1), but a noncognate tRNA, such as isoleucine, (SEQ ID NO: 2) may be bound under certain conditions. In another embodiment, the tRNA molecule is a leucyl t-RNA. In another embodiment, the tRNA molecule is represented by a SEQ ID described herein. In another embodiment, the tRNA molecule is selected from the group consisting of SEQ ID NO: 14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23 and SEQ ID NO:24. In this and other embodiments, the term “noncognate” is meant to encompass both the singular and plural forms of the word, i.e. the phrase “noncognate amino acid” comprises one or more amino acids. In the following sequences; s4U=s⁴U; 4-thiouridine; Gm=methylguanine; Y=pyrimidine; ms2i6A=ms²i⁶A; 2-methylthio-N-6-isopentenyl adenosine and D=dihydrouridine.

corresponds to the nucleotide sequence of the
tRNA-Leu gene from Saccharomyces cerevisiae:
SEQ ID NO: 1
gggagtttgg ccgagtggtt taaggcgtca gatttaggct
ctgatatctt cggatgcaagggttcgaatc ccttagctct cacca
corresponds to the nucleotide sequence of the
tRNA-Ilegene from Saccharomyces cerevisiae:
SEQ ID NO: 2
gaaactataa ttcaattggt tagaatagta ttttgataag
gtacaaatat aggttcaatc cctgttagtt tcatcca
corresponds to the nucleotide sequence of a
tRNA-Leu gene from E. coli:
SEQ ID NO: 14
gcgaaggtggcggaattggtagacgcgctagcttcaggtgttagtgtcc
ttacggacgtgggggttcaagtcccccccctcgcacca
corresponds to the nucleotide sequence of a
tRNA-Leu gene from E. coli:
SEQ ID NO: 15
gcgggagtggcgaaattggtagacgcaccagatttaggttctggcgccg
caaggtgtgcgagttcaagtctcgcctcccgcacca
corresponds to the nucleotide sequence of a
tRNA-Leu gene from E. coli:
SEQ ID NO: 16
gccgaagtggcgaaatcggtagacgcagttgattcaaaatcaaccgtag
aaatacgtgccggttcgagtccggccttcggcacca
corresponds to the nucleotide sequence of a
tRNA-Leu gene from E. coli:
SEQ ID NO: 17
gccgaggtggtggaattggtagacacgctaccttgaggtggtagtgccc
aatagggcttacgggttcaagtcccgtcctcggtacca
corresponds to the nucleotide sequence
of a tRNA-Leu gene from E. coli:
SEQ ID NO: 18
gcccggatggtggaatcggtagacacaagggatttaaaatccctcggcg
ttcgcgctgtgcgggttcaagtcccgctccgggtacca
corresponds to the nucleotide sequence of a
tRNA-Leu gene from E. coli:
SEQ ID NO: 19
GCCCGGAs4UGGUGGAADCGmGDAGACACAAGGGAYUunkAAAms2i6A
AYCCCUCGGCGUUCGCGCUGUGCGGGTYCAAGUCCCGCUCCGGGUACCA
corresponds to the nucleotide sequence
of a tRNA-Leu gene from E. coli:
SEQ ID NO: 20
GCGAAGGUGGCGGAADDGmGDAGACGCGCUAGCUUCAGunkGYGYUAGU
GUCCUUACGGACGUGGGGGTYCAAGUCCCCCCCCUCGCACCA
corresponds to the nucleotide sequence of a
tRNA-Leu gene from E. coli:
SEQ ID NO: 21
GCCGAGGUGGUGGAADDGmGDAGACACGCUACCUUGAGunkGYGGUAGU
GCCCAAUAGGGCUUACGGGTYCAAGUCCCGUCCUCGGUACCA
corresponds to the nucleotide sequence of a
tRNA-Leu gene from Pseudomonas aeruginosa
SEQ ID NO: 22
gcggacgtggtggaattggtagacacactggatttaggttccagcgccg
caaggcgtgagagttcgagtctctccgtccgcacca
corresponds to the nucleotide sequence of a
tRNA-Leu gene from Staphylococcus aureus
SEQ ID NO: 23
gccggggtggcggaactggcagacgcacaggacttaaaatcctgcggtg
agagatcaccgtaccggttcgattccggtcctcggcacca
corresponds to the nucleotide sequence of a
tRNA-Leu gene from Staphylococcus aureus
SEQ ID NO: 24
gccggggtggcggaactggcagacgcacaggacttaaaatcctgcggtg
agtgatcaccgtaccggttcgattccggtcctcggcacca

Polypeptides Used in Binding and Inhibition Assays

In some binding and inhibition assays, it is more effective to use a portion of a tRNA synthetase molecule rather than the whole protein itself. In such assays, polypeptides derived from tRNA synthetases are used in the experiment.

In one preferred embodiment, polypeptide fragments corresponding to the editing domain of a tRNA synthetase molecule are used in assay and binding experiments. Such fragments are selected from the group consisting of SEQ ID NO:3, SEQ ID NO:4, SEQ ID NO:5, SEQ ID NO:6 and SEQ ID NO:7. In an exemplary embodiment, the fragments are selected from the group consisting of SEQ ID NO:5, SEQ ID NO:6 and SEQ ID NO:7.

SEQ ID NO 3:
TPQEYIGVKIEALEFADDAAKIIDSSSDLDKSKKFYFVAATLRPETMYG
QTCCFVSPTIEYGIFDAGDSYFITTERAFKNMSYQKLTPKRGFYKPIVT
VPGKAFIGTKIHAPQSVYPELRILPMETVIATKGTGVVTCVPSNSPDDY
ITTKDLLHKPEYYGIKPEWIDHEIVPIMHTEKYGDLTAKAIVEEKKIQS
PKDKNLLAEAKKIAYKEDYYTGTMIYGPYKGEKVEQAKNKVKADMIAAG
EAFVYNEPESQDP
SEQ ID NO 4:
MTPQEYIGVKIEALEFADDAAKIIDSSSDLDKSKKFYFVAATLRPETMY
GQTCCFVSPTIEYGIFDAGDSYFITTERAFKNMSYQKLTPKRGFYKPIV
TVPGKAFIGTKIHAPQSVYPELRILPMETVIATKGTGVVTCVPSNSPDD
YITTKDLLHKPEYYGIKPEWIDHEIVPIMHTEKYGDLTAKAIVEEKKIQ
SPKDKNLLAEAKKIAYKEDYYTGTMIYGPYKGEKVEQAKNKVKADMIAA
GEAFVYNEPESQDPQDPNSSSVDKLAAALEHHHHH
SEQ ID NO 5:
TCTPEYYRWEQKFFTELYKKGLVYKKTSAVNWCPNDQTVLANEQVIDGC
CWRCDTKVERKEIPQWFIKITAYADELLNDLDKLDHWPDTVKTMQRNWI
GRSEGVEITFNVNDYDNTLTVYTTRPDTFMGCTYLAVAAGHPLAQKAAE
NNPELAAFIDECRNTKVAEAEMATMEKKGVDTGFKAVHPLTGEEIPVWA
ANFVLMEYGTGAVMAVPGHDQRDYEFASKYGLNIKPVILAADGSEPDLS
QQALTEKGVLFNSGEFNGLDHEAAFNAIADKLTAMGVGERKVNYRLRDW
GVSRQRYWG
SEQ ID NO 6:
TCKPDYYRWEQWLFTRLFEKGVIYRKNGTVNWDPADQTVLANEQVIDGR
GWRSGALIEKREIPMYYFRITDYADELLESLDELPGWPEQVKTMQRNWI
GKSRGMEVQFPYDQASIGHEGTLKVFTTRPDTLMGATYVAVAAEHPLAT
QAAQGNAALQAFIDECKSGSVAEADMATQEKKGMATSLFVEHPLTGEKL
PVWVANYVLMHYGDGAVMAVPAHDERDFEFAHKYNLPVKAVVRTSAGDD
VGSEWLAAYGEHGQLINSGEFDGLDFQGAFDAIEAALIRKDLGKSRTQF
RLRDWGISRQRYWG
SEQ ID NO 7:
TTDPEYYKWTQWIFIQLYNKGLAYVDEVAVNWCPALGTVLSNEEVIDGV
SERGGHPVYRKPMKQWVLKITEYADQLLADLDDLDWPESLKDMQRNWIG
RSEGAKVSFDVDNTEGKVEVFTTRPDTIYGASFLVLSPEHALVNSITTD
EYKEKVKAYQTEASKKSDLERTDLAKDKSGVFTGAYAINPLSGEKVQIW
IADYVLSTYGTGAIMAVPAHDDRDYEFAKKFDLLIIEVIEGGNVEEAAY
TGEGKHINSGELDGLENEAAITKAIQLLEQKGAGEKKVYKLRDWLFSRQ
RYWG
corresponds to a peptide sequence for a leu-tRNA
synthetase editing domain for Escherichia coli
SEQ ID NO 8
GRSEGVEITFNVNDYDNTLTVYTTRPDTFMGCTYLAVAAGHPLAQKAAE
NNPELAAFIDECRNTKVAEAEMATMEKKGVDTGFKAVHPLTGEEIPVWA
ANFVLMEYGTGAVMAVPGHDQRDYEFASKYGLNIKPVILAADGSEPDLS
QQALTEKGVLFNSGEFNGLDHEAAFNAIADKLTAMGVGERKVNYR
corresponds to a peptide sequence for a leu-tRNA
synthetase editing domain for Pseudomonas
SEQ ID NO 9
GKSRGMEVQFPYDQASIGHEGTLKVFTTRPDTLMGATYVAVAAEHPLAT
QAAQGNAALQAFIDECKSGSVAEADMATQEKKGMATSLFVEHPLTGEKL
PVWVANYVLMHYGDGAVMAVPAHDERDFEFAHKYNLPVKAVVRTSAGDD
VGSEWLAAYGEHGQLINSGEFDGLDFQGAFDAIEAALIRKDLGKSRTQF
R
corresponds to a peptide sequence for a leu-tRNA
synthetase editing domain for Staphylococcus
aureus
SEQ ID NO 10
GRSEGAKVSFDVDNTEGKVEVFTTRPDTIYGASFLVLSPEHALVNSITT
DEYKEKVKAYQTEASKKSDLERTDLAKDKSGVFTGAYAINPLSGEKVQI
WIADYVLSTYGTGAIMAVPAHDDRDYEFAKKFDLLIIEVIEGGNVEEAA
YTGEGKHINSGELDGLENEAAITKAIQLLEQKGAGEKKVYK

In one preferred embodiment, polypeptides corresponding to a tRNA synthetase molecule are used in assay and binding experiments. Such polypeptides are selected from the group consisting of SEQ ID NO:11, SEQ ID NO:12 and SEQ ID NO:13.

corresponds to a peptide sequence for a leu-tRNA
synthetase for Escherichia coli
SEQ ID NO 11
MQEQYRPEEIESKVQLHWDEKRTFEVTEDESKEKYYCLSMLPYPSGRLH
MGHVRNYTIGDVIARYQRMLGKNVLQPIGWDAFGLPAEGAAVKNNTAPA
PWTYDNIAYMKNQLKMLGFGYDWSRELATCTPEYYRWEQKFFTELYKKG
LVYKKTSAVNWCPNDQTVLANEQVIDGCCWRCDTKVERKEIPQWFIKIT
AYADELLNDLDKLDHWPDTVKTMQRNWIGRSEGVEITFNVNDYDNTLTV
YTTRPDTFMGCTYLAVAAGHPLAQKAAENNPELAAFIDECRNTKVAEAE
MATMEKKGVDTGFKAVHPLTGEEIPVWAANFVLMEYGTGAVMAVPGHDQ
RDYEFASKYGLNIKPVILAADGSEPDLSQQALTEKGVLFNSGEFNGLDH
EAAFNAIADKLTAMGVGERKVNYRLRDWGVSRQRYWGAPIPMVTLEDGT
VMPTPDDQLPVILPEDVVMDGITSPIKADPEWAKTTVNGMPALRETDTF
DTFMESSWYYARYTCPQYKEGMLDSEAANYWLPVDIYIGGIEHAIMHLL
YFRFFHKLMRDAGMVNSDEPAKQLLCQGMVLADAFYYVGENGERNWVSP
VDAIVERDEKGRIVKAKDAAGHELVYTGMSKMSKSKNNGIDPQVMVERY
GADTVRLFMMFASPADMTLEWQESGVEGANRFLKRVWKLVYEHTAKGDV
AALNVDALTENQKALRRDVHKTIAKVTDDIGRRQTFNTAIAAIMELMNK
LAKAPTDGEQDRALMQEALLAVVRMLNPFTPHICFTLWQELKGEGDIDN
APWPVADEKAMVEDSTLVVVQVNGKVRAKITVPVDATEEQVRERAGQEH
LVAKYLDGVTVRKVIYVPGKLLNLVVG
corresponds to a peptide sequence for a leu-tRNA
synthetase for Pseudomonas
SEQ ID NO 12
MHEQYTPRDVEAAAQNAWDEQQSFAVTEQPGKETYYCLSMFPYPSGKLH
MGHVRNYTIGDVIARYQRMLGKNVLQPMGWDAFGMPAENAAMKNNVAPA
KWTYENIDYMKTQLKSLGLAIDWSREVTTCKPDYYRWEQWLFTRLFEKG
VIYRKNGTVNWDPADQTVLANEQVIDGRGWRSGALIEKREIPMYYFRIT
DYADELLESLDELPGWPEQVKTMQRNWIGKSRGMEVQFPYDQASIGHEG
TLKVFTTRPDTLMGATYVAVAAEHPLATQAAQGNAALQAFIDECKSGSV
AEADMATQEKKGMATSLFVEHPLTGEKLPVWVANYVLMHYGDGAVMAVP
AHDERDFEFAHKYNLPVKAVVRTSAGDDVGSEWLAAYGEHGQLINSGEF
DGLDFQGAFDAIEAALIRKDLGKSRTQFRLRDWGISRQRYWGCPIPIIH
CPSCGDVPVPEDQLPVTLPENVVPDGAGSPLARMPEFYECTCPKCGTAA
KRETDTMDTFVESSWYFARYASPNYDKGLVDPKAANHWLPVDQYIGGIE
HAILHLLYARFFHKLMRDEGLVTSNEPFKNLLTQGMVVAETYYRVASNG
GKDWFNPADVEIERDAKAKIIGARLKTDGLPVEIGGTEKMSKSKNNGVD
PQSMIEQYGADTCRLFMMFASPPDMSLEWSDSGVEGASRFLRRVWRLAQ
AHVAQGLPGQLDIAALSDEQKVIRRAIHAAIKQASTDVGQFHKFNTAIA
QVMTVMNVLEKAPQVTAQDRALLQEGLEAVTLLLAPITPHISHELWKQL
GHEQAVIDATWPSVDESALVQDTVTLVVQVNGKLRGQVEMPAAASREEI
EAAARNNENVLRFTDGLTIRKVIVVPGKLVNIVAN
corresponds to a peptide sequence for a leu-tRNA
synthetase for Staphylococcus aureus
SEQ ID NO 13
MNYNHNQIEKKWQDYWDENKTFKTNDNLGQKKFYALDMFPYPSGAGLHV
GHPEGYTATDIISRYKRMQGYNVLHPMGWDAFGLPAEQYALDTGNDPRE
FTKKNIQTFKRQIKELGFSYDWDREVNTTDPEYYKWTQWIFIQLYNKGL
AYVDEVAVNWCPALGTVLSNEEVIDGVSERGGHPVYRKPMKQWVLKITE
YADQLLADLDDLDWPESLKDMQRNWIGRSEGAKVSFDVDNTEGKVEVFT
TRPDTIYGASFLVLSPEHALVNSITTDEYKEKVKAYQTEASKKSDLERT
DLAKDKSGVFTGAYAINPLSGEKVQIWIADYVLSTYGTGAIMAVPAHDD
RDYEFAKKFDLLIIEVIEGGNVEEAAYTGEGKHINSGELDGLENEAAIT
KAIQLLEQKGAGEKKVNYKLRDWLFSRQRYWGEPIPVIHWEDGTMTTVP
EEELPLLLPETDEIKPSGTGESPLANIDSFVNVVDEKTGMKGRRETNTM
PQWAGSCWYYLRYIDPKNENMLADPEKLKHWLPVDLYIGGVEHAVLHLL
YARFWHKVLYDLGIVPTKEPFQKLFNQGMILGEGNEKMSKSKGNVINPD
DIVQSHGADTLRLYEMFMGPLDAAIAWSEKGLDGSRRFLDRVWRLIVNE
DGTLSSKIVTTNNKSLDKVYNQTVKKVTDDFETLGFNTAISQLMVFINE
CYKVDEVYKPYIEGFVKMLAPIAPHIGEELWSKLGHEESITYQPWPTYD
EALLVDDEVEIVVQVNGKLRAKIKIAKDTSKEEMQEIALSNDNVKASIE
GKDIMKVIAVPQKLVNIVAK

### VI. Methods for Inhibiting an Enzyme

The compounds of the invention can be utilized to inhibit an enzyme. In an exemplary embodiment, the compounds of the invention exhibit the ability of inhibiting the editing domain of tRNA synthetases, such as leucyl tRNA synthetase, of microorganisms, such as bacteria, and therefore have the potential to be used as editing domain inhibitors of microorganism tRNA synthetases.

According to another aspect of the invention, a method for binding to and/or inhibiting the editing domain of a tRNA synthetase is provided which comprises contacting a tRNA synthetase with a compound of the invention that inhibits the editing domain under conditions in which the tRNA synthetase interacts with its substrate to form an aminoacyl adenylate intermediate and, preferably, to form a charged tRNA. Such conditions are known to those skilled in the art. In an exemplary embodiment, the compound is described herein, or a salt, hydrate or solvate thereof, or a combination thereof. In an exemplary embodiment, the invention provides a compound described herein, or a salt, hydrate or solvate thereof. In an exemplary embodiment, the invention provides a compound described herein, or a salt thereof. In an exemplary embodiment, the invention provides a compound described herein, or a salt thereof. The tRNA synthetase is contacted with an amount of compound of the invention sufficient to result in a detectable amount of tRNA synthetase inhibition. This method can be performed on a tRNA synthetase that is contained within an organism or which is outside an organism. In an exemplary embodiment, the method is performed on a tRNA synthetase that is contained within a microorganism or a microbial cell that is in, or on the surface of, an animal. In an exemplary embodiment, the animal is a human. The method results in a decrease in the amount of charged tRNA produced by the tRNA synthetase that has an inhibited editing domain. In an exemplary embodiment, the inhibition takes place in a cell, such as a microorganism cell. In another exemplary embodiment, the microorganism cell is a bacteria. In another exemplary embodiment, the tRNA synthetase is a mitochondrial tRNA synthetase or a cytoplasmic tRNA synthetase. In another exemplary embodiment, the tRNA synthetase is selected from the group consisting of alanyl tRNA synthetase, isoleucyl tRNA synthetase, leucyl tRNA synthetase, methionyl tRNA synthetase, lysyl tRNA synthetase, phenylalanyl tRNA synthetase, prolyl tRNA synthetase, threonyl tRNA synthetase and valyl tRNA synthetase. In another exemplary embodiment, the tRNA synthetase is leucyl tRNA synthetase.

In an exemplary embodiment, the invention provides a method of inhibiting conversion of a tRNA molecule into a charged tRNA molecule. The method involves contacting a tRNA synthetase with a compound of the invention effective to inhibit activity of an editing domain of said tRNA synthetase, under conditions sufficient to inhibit said activity, thereby inhibiting said conversion. In an exemplary embodiment, the compound of the invention is a compound described herein, or a pharmaceutically acceptable salt thereof. In an exemplary embodiment, the inhibition occurs within a cell, and the cell is a microorganism cell. In another exemplary embodiment, the microorganism cell is a bacteria. In another exemplary embodiment, the microorganism cell is a bacteria which is described herein. In another exemplary embodiment, the enzyme is a leucyl tRNA synthetase of a bacteria described herein. In an exemplary embodiment, the tRNA synthetase is a mitochondrial tRNA synthetase or a cytoplasmic tRNA synthetase. In another exemplary embodiment, the tRNA synthetase selected from the group consisting of alanyl tRNA synthetase, isoleucyl tRNA synthetase, leucyl tRNA synthetase, methionyl tRNA synthetase, lysyl tRNA synthetase, phenylalanyl tRNA synthetase, prolyl tRNA synthetase, threonyl tRNA synthetase and valyl tRNA synthetase. In another exemplary embodiment, the tRNA synthetase is leucyl tRNA synthetase. In another exemplary embodiment, the compound has a K_{D, synthesis} of greater than 100 μM against a synthetic domain of said tRNA synthetase.

In certain embodiments, the mechanism of action of a compound of the invention is to inhibit the conversion of a tRNA molecule into a charged tRNA molecule by binding to and/or inhibiting at least the editing domain of the synthetase. The compounds of use in this method may also inhibit or otherwise interact with the synthetic domain (e.g., the active site of the synthetic domain). In a presently preferred embodiment, the editing domain is inhibited selectively in the presence of the synthetic domain. In a preferred embodiment, the synthetic domain is essentially uninhibited, while the editing domain is inhibited at least 50%, preferably at least 60%, more preferably at least 70%, still more preferably, at least 80% and even still more preferably at least 90% of the activity of the tRNA synthetase. In another preferred embodiment, the synthetic domain is inhibited by at most 50%, preferably at most 30%, preferably at most 20%, 10%, preferably at most 8%, more preferably at most 5%, still more preferably, at most 3% and even still more preferably at most 1%. Inhibition of the editing domain produces a decrease in the amount of the properly charged tRNA which results in retardation or cessation of cell growth and division.

In another exemplary embodiment, the ratio of a minimum concentration of said compound inhibiting said editing domain to a minimum concentration of said compound inhibiting said synthetic domain of said tRNA synthetase, represented as K_{D, edit}/K_{D, synthesis}, is less than one. In another exemplary embodiment, the K_{D, edit}/K_{D, synthesis} of the compound is selected from the group consisting of less than 0.5, less than 0.1 and less than 0.05.

### VII. Methods of Inhibiting Microorganism Growth or Killing Microorganisms

The compounds of the invention exhibit potency against microorganisms, such as bacteria, and therefore have the potential to kill and/or inhibit the growth of microorganisms.

In a further aspect, the invention provides a method of killing and/or inhibiting the growth of a microorganism, said method comprising: contacting said microorganism with an effective amount of a compound of the invention, thereby killing and/or inhibiting the growth of the microorganism. In an exemplary embodiment, the microorganism is a bacteria. In an exemplary embodiment, the compound is described herein, or a salt, prodrug, hydrate or solvate thereof, or a combination thereof. In an exemplary embodiment, the invention provides a compound described herein, or a salt, hydrate or solvate thereof. In an exemplary embodiment, the invention provides a compound described herein, or a prodrug thereof. In an exemplary embodiment, the invention provides a compound described herein, or a salt thereof. In another exemplary embodiment, the compound of the invention is a compound described herein, or a pharmaceutically acceptable salt thereof. In another exemplary embodiment, the compound is described by a formula listed herein, or a pharmaceutically acceptable salt thereof. In an exemplary embodiment, the compound is part of a pharmaceutical formulation described herein. In another exemplary embodiment, the contacting occurs under conditions which permit entry of the compound into the organism. In an exemplary embodiment, the compound inhibits the tRNA synthetase through the editing domain of the synthetase. Such conditions are known to one skilled in the art and specific conditions are set forth in the Examples appended hereto. This method involves contacting a microorganism with a therapeutically-effective amount of an editing domain inhibitor to inhibit tRNA synthetase in vivo or in vitro. In another exemplary embodiment, R* is H.

In another aspect, the microorganism is inside, or on the surface of an animal. In an exemplary embodiment, the animal is selected from the group consisting of human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human.

In an exemplary embodiment, the microorganism is killed or its growth is inhibited through oral administration of the compound of the invention. In an exemplary embodiment, the microorganism is killed or its growth is inhibited through intravenous administration of the compound of the invention.

In an exemplary embodiment, the microorganism is a bacterium. In an exemplary embodiment, the bacterium is a gram-positive bacteria. In another exemplary embodiment, the gram-positive bacterium is selected from the group consisting of Staphylococcus species, Streptococcus species, Bacillus species, Mycobacterium species, Corynebacterium species (Propionibacterium species), Clostridium species, Actinomyces species, Enterococcus species and Streptomyces species. In another exemplary embodiment, the gram-positive bacterium is selected from the group consisting of Propionibacterium acnes; Staphylococcus aureus; Staphylococcus epidermidis, Staphylococcus saprophyticus; Staphylococcus haemolyticus; Streptococcus pyogenes; Streptococcus agalactiae; Streptococcus pneumoniae; Enterococcus faecalis; Enterococcus faecium; Bacillus anthracis; Mycobacterium avium-intracellulare; Mycobacterium tuberculosis, Corynebacterium diphtheria; Clostridium perfringens; Clostridium botulinum; Clostridium tetani; and Clostridium difficile. In another exemplary embodiment, the gram-positive bacterium is selected from the group consisting of Staphylococcus aureus, Staphylococcus epidermidis, Streptococcus pneumoniae, Streptococcus pyogenes, Enterococcus faecalis, Enterococcus faecium, Clostridium difficile and Propionibacter acnes. In another exemplary embodiment, the bacterium is a gram-negative bacterium. In another exemplary embodiment, the gram-negative bacterium is selected from the group consisting of Acinetobacter species, Neisseria species, Pseudomonas species, Brucella species, Agrobacterium species, Bordetella species, Escherichia species, Shigelia species, Yersinia species, Salmonella species, Klebsiella species, Enterobacter species, Haemophilus species, Pasteurella species, Streptobacillus species, spirochetal species, Campylobacter species, Vibrio species, Helicobacter species, Bacteroides species, Citrobacter species, Proteus species, Providencia species, Serratia species, Stenotrophomonas species and Burkholderia species. In another exemplary embodiment, the gram-negative bacterium is selected from the group consisting of Acinetobacter species, Pseudomonas species, Escherichia species, Klebsiella species, Enterobacter species, Bacteroides species, Citrobacter species, Proteus species, Providencia species, Serratia species, Stenotrophomonas species and Burkholderia species. In another exemplary embodiment, the gram-negative bacterium is selected from the group consisting of Neisseria gonorrhoeae; Neisseria meningitidis; Pseudomonas aeruginosa; Legionella pneumophila; Escherichia coli; Yersinia pestis; Haemophilus influenzae; Helicobacter pylori; Campylobacter fetus; Campylobacter jejuni; Vibrio cholerae; Vibrio parahemolyticus; Trepomena pallidum; Actinomyces israelii; Rickettsia prowazekii; Rickettsia rickettsii; Chlamydia trachomatis; Chlamydia psittaci; Brucella abortus; Agrobacterium tumefaciens; Francisella tularensis, Klebsiella pneumoniae, Enterobacter cloacae, Acinetobacter baumannii, Bacteroides fragilis, Citrobacter freundii, Proteus mirabilis, Providencia stuartii, Serratia marcescens, Stenotrophomonas maltophilia and Burkholderia cepacia. In another exemplary embodiment, the gram-negative bacterium is selected from the group consisting of Pseudomonas aeruginosa; Escherichia coli; Haemophilus influenzae, Klebsiella pneumoniae, Enterobacter cloacae, Acinetobacter baumannii, Bacteroides fragilis, Citrobacter freundii, Proteus mirabilis, Providencia stuartii, Serratia marcescens, Stenotrophomonas maltophilia and Burkholderia cepacia. In another exemplary embodiment, the gram-negative bacterium is selected from the group consisting of Enterobacter aerogenes; Enterobacter cloacae; Enterobacter sakazakii; Escherichia coli; Klebsiella pneumoniae; Proteus mirabilis; Serratia marcescens and Citrobacter freundii. In another exemplary embodiment, the gram-negative bacterium is a Providencia spp. In another exemplary embodiment, the gram-negative bacterium is an Enterobacter spp.

In another exemplary embodiment, the bacterium is a Pseudomonas species. In another exemplary embodiment, the bacterium is Pseudomonas aeruginosa. In another exemplary embodiment, the bacterium is selected from the group consisting of Pseudomonas aeruginosa; Acinetobacter baumannii, Stenotrophomonas maltophilia and Burkholderia cepacia. In another exemplary embodiment, the bacterium is Acinetobacter baumannii. In another exemplary embodiment, the bacterium is Stenotrophomonas maltophilia. In another exemplary embodiment, the bacterium is Burkholderia cepacia. In another exemplary embodiment, the bacterium is Acinetobacter species. In another exemplary embodiment, the bacterium is Acinetobacter anitratus. In another exemplary embodiment, the bacterium is selected from the group consisting of Enterobacter aerogenes, Enterobacter cloacae, Enterobacter sakazakii, E. coli, K. pneumoniae, P. mirabilis, Serratia marcescens, Citrobacter freundii and Providencia spp. In another exemplary embodiment, the bacterium is selected from the group consisting of Enterobacter aerogenes, Enterobacter cloacae, Enterobacter sakazakii, E. coli, K. pneumoniae, P. mirabilis, Serratia marcescens, Citrobacter freundii, Providencia spp., S. aureus, S. pneumonia, S. pyogenes, E. faecalis, and E. faecium. In another exemplary embodiment, the bacterium is selected from the group consisting of Pseudomonas aeruginosa; Acinetobacter baumannii; Stenotrophomonas maltophilia and Burkholderia cepacia. In another exemplary embodiment, the bacterium is selected from the group consisting of S. aureus, S. pneumonia, S. pyogenes, E. faecalis, and E. faecium. In another exemplary embodiment, the bacterium is Viridans group Strep. In another exemplary embodiment, the bacterium is selected from the group consisting of Strep. mitis, Strep. mutans, Strep. oralis, Strep. sanguis, Strep. sobrinus and Strep. millari. In another exemplary embodiment, the bacterium is S. pneumonia. In another exemplary embodiment, the bacterium is H. influenzae. In another exemplary embodiment, the bacterium is S. aureus. In another exemplary embodiment, the bacterium is M. catarrhalis. In another exemplary embodiment, the bacterium is M. pneumoniae. In another exemplary embodiment, the bacterium is L. pneumoniae. In another exemplary embodiment, the bacterium is C. pneumoniae. In another exemplary embodiment, the bacterium is S. pyogenes. In another exemplary embodiment, the bacterium is an anaerobe. In another exemplary embodiment, the bacterium is an Alcaligenes species. In another exemplary embodiment, the bacterium is a B. cepacia. In another exemplary embodiment, the bacterium is selected from the group consisting of Enterobacter cloacae, Escherichia coli; Klebsiella pneumoniae, Proteus mirabilis, Providencia stuartii, Serratia marcescens, and Citrobacter freundii. In another exemplary embodiment, the bacterium is resistant to methicillin. In another exemplary embodiment, the bacterium is methicillin-resistant staphylococcus aureus. In another exemplary embodiment, the bacterium is selected from the group consisting of Streptococcus pneumoniae; Haemophilus influenzae; Staphylococcus aureus; Mycobacterium catarrhalis; Mycobacterium pneumoniae; Legionella pneumophila and Chlamydia pneumoniae. In another exemplary embodiment, the bacterium is selected from the group consisting of Enterobacter cloacae, Escherichia coli; Klebsiella pneumoniae, Proteus mirabilis, Serratia marcescens, Citrobacter freundii, Providencia stuartii, Pseudomonas aeruginosa; Acinetobacter baumannii, Stenotrophomonas maltophilia, Burkholderia cepacia, Staphylococcus aureus; Streptococcus pneumoniae; Streptococcus pyogenes; Enterococcus faecalis; and Enterococcus faecium. In another exemplary embodiment, the bacterium is selected from the group consisting of Staphylococcus aureus; Staphylococcus epidermidis, Staphylococcus haemolyticus; Streptococcus pyogenes; Streptococcus agalactiae and Streptococcus pneumoniae.

In an exemplary embodiment, the microorganism is a bacterium, which is selected from the group consisting of acid-fast bacteria, including Mycobacterium species; bacilli, including Bacillus species, Corynebacterium species (also Propionibacterium) and Clostridium species; filamentous bacteria, including Actinomyces species and Streptomyces species; bacilli, such as Pseudomonas species, Brucella species, Agrobacterium species, Bordetella species, Escherichia species, Shigella species, Yersinia species, Salmonella species, Klebsiella species, Enterobacter species, Haemophilus species, Pasteurella species, and Streptobacillus species; spirochetal species, Campylobacter species, Vibrio species; and intracellular bacteria including Rickettsiae species and Chlamydia species. In an exemplary embodiment, the microorganism is described in a FIGURE provided herein.

### VIII. Methods of Treating and/or Preventing Disease

The compounds of the invention exhibit potency against microorganisms, such as bacteria, and therefore have the potential to achieve therapeutic efficacy in the animals described herein.

In another aspect, the invention provides a method of treating and/or preventing a disease. The method includes administering to the animal a therapeutically effective amount of the compound of the invention, sufficient to treat and/or prevent the disease. In an exemplary embodiment, the compound of the invention can be used in human or veterinary medical therapy, particularly in the treatment or prophylaxis of bacterial-associated disease. In an exemplary embodiment, the compound is described herein, or a salt, prodrug, hydrate or solvate thereof, or a combination thereof. In an exemplary embodiment, the invention provides a compound described herein, or a prodrug thereof. In an exemplary embodiment, the invention provides a compound described herein, or a salt, hydrate or solvate thereof. In an exemplary embodiment, the invention provides a compound described herein, or a salt thereof. In another exemplary embodiment, the compound of the invention is a compound described herein, or a pharmaceutically acceptable salt thereof. In an exemplary embodiment, the compound is a compound described herein, or a pharmaceutically acceptable salt thereof. In an exemplary embodiment, the compound is according to a formula described herein, or a pharmaceutically acceptable salt thereof. In an exemplary embodiment, the compound is part of a pharmaceutical formulation described herein. In another exemplary embodiment, the animal is selected from the group consisting of human, cattle, deer, reindeer, goat, honey bee, pig, sheep, horse, cow, bull, dog, guinea pig, gerbil, rabbit, cat, camel, yak, elephant, ostrich, otter, chicken, duck, goose, guinea fowl, pigeon, swan, and turkey. In another exemplary embodiment, the animal is a human. In another exemplary embodiment, the animal is selected from the group consisting of a human, cattle, goat, pig, sheep, horse, cow, bull, dog, guinea pig, gerbil, rabbit, cat, chicken and turkey. In another exemplary embodiment, the disease is selected from the group consisting of a systemic disease, a cutaneous disease, an ungual disease, periungual disease and subungual disease. In another exemplary embodiment, the disease is a systemic disease. In another exemplary embodiment, R* is H.

In another exemplary embodiment, the treatment of a disorder or condition occurs through inhibition of an editing domain of an aminoacyl tRNA synthetase. In an exemplary embodiment, the disease is treated through oral administration of the compound of the invention. In an exemplary embodiment, the disease is treated through intravenous administration of the compound of the invention.

### VIII.a) Methods of Treating Systemic Diseases

In another aspect, the invention provides a method of treating a systemic disease. The method involves contacting an animal with a compound of the invention.

In an exemplary embodiment, the disease is selected from the group consisting of candidiasis, aspergillosis, coccidioidomycosis, cryptococcosis, histoplasmosis, blastomycosis, paracoccidioidomycosis, zygomycosis, phaeohyphomycosis and rhinosporidiosis.

In an exemplary embodiment, the disease is associated with an infection by a microorganism described herein. In an exemplary embodiment, the disease is associated with an infection by a bacterium described herein.

In another exemplary embodiment, the disease is associated with infection by a Gram-positive bacteria. In an exemplary embodiment, the disease is associated with a Staphylococcus species. In another exemplary embodiment, the disease is selected from the group consisting of pneumonia, gastroenteritis, toxic shock syndrome, CAP, meningitis, septic arthritis, urinary tract infections, bacteremia, endocarditis, osteomylitis, skin and skin-structure infections. In an exemplary embodiment, the disease is associated with a Streptococcus species. In another exemplary embodiment, the disease is selected from the group consisting of strep throat, skin infections, necrotizing fasciitis, toxic shock syndrome, pneumonia, otitis media and sinusitis. In an exemplary embodiment, the disease is associated with an Actinomyces species. In another exemplary embodiment, the disease is actinomycosis. In an exemplary embodiment, the disease is associated with a Norcardia species. In another exemplary embodiment, the disease is pneumonia. In an exemplary embodiment, the disease is associated with a Corynebacterium species. In another exemplary embodiment, the disease is diphtheria. In an exemplary embodiment, the disease is associated with a Listeria species. In another exemplary embodiment, the disease is meningitis. In an exemplary embodiment, the disease is associated with a Bacillus species. In another exemplary embodiment, the disease is anthrax or food poisoning. In an exemplary embodiment, the disease is associated with a Clostridium species. In another exemplary embodiment, the disease is selected from the group consisting of botulism, tetanus, gas gangrene and diarrhea. In an exemplary embodiment, the disease is associated with a Mycobacterium species. In another exemplary embodiment, the disease is tuberculosis or leprosy.

In another exemplary embodiment, the disease is associated with infection by a Gram-negative bacteria. In an exemplary embodiment, the disease is associated with a Neisseria species. In another exemplary embodiment, the disease is selected from the group consisting of meningitis, gonorrhea, otitis extema and folliculitis. In an exemplary embodiment, the disease is associated with an Escherichia species. In another exemplary embodiment, the disease is selected from the group consisting of diarrhea, urinary tract infections, meningitis, sepsis and HAP. In an exemplary embodiment, the disease is associated with a Shigella species. In another exemplary embodiment, the disease is selected from the group consisting of diarrhea, bacteremia, endocarditis, meningitis and gastroenteritis. In an exemplary embodiment, the disease is associated with a Salmonella species. In another exemplary embodiment, the disease is selected from the group consisting of Typhoid fever, sepsis, gastroenteritis, endocarditis, sinusitis and meningitis. In an exemplary embodiment, the disease is associated with a Yersinia species. In another exemplary embodiment, the disease is selected from the group consisting of Typhoid fever, bubonic plague, enteric fever and gastroenteritis. In an exemplary embodiment, the disease is associated with a Klebsiella species. In another exemplary embodiment, the disease is sepsis or urinary tract infection. In an exemplary embodiment, the disease is associated with a Proteus species. In another exemplary embodiment, the disease is an urinary tract infection. In an exemplary embodiment, the disease is associated with an Enterobacter species. In another exemplary embodiment, the disease is a hospital-acquired infection. In an exemplary embodiment, the disease is associated with a Serratia species. In another exemplary embodiment, the disease is selected from the group consisting of an urinary tract infection, skin and skin-structure infection and pneumonia. In an exemplary embodiment, the disease is associated with a Vibrio species. In another exemplary embodiment, the disease is cholera or gastroenteritis. In an exemplary embodiment, the disease is associated with a Campylobacter species. In another exemplary embodiment, the disease is gastroenteritis. In an exemplary embodiment, the disease is associated with a Helicobacter species. In another exemplary embodiment, the disease is chronic gastritis. In an exemplary embodiment, the disease is associated with a Pseudomonas species. In another exemplary embodiment, the disease is selected from the group consisting of pneumonia, osteomylitis, burn-wound infections, sepsis, UTIs, endocarditis, otitis and corneal infections. In an exemplary embodiment, the disease is associated with a Bacteroides species. In another exemplary embodiment, the disease is periodontal disease or aspiration pneumonia. In an exemplary embodiment, the disease is associated with a Haemophilus species. In another exemplary embodiment, the disease is selected from the group consisting of meningitis, epiglottitis, septic arthritis, sepsis, chancroid and vaginitis. In an exemplary embodiment, the disease is associated with a Bordetella species. In another exemplary embodiment, the disease is whooping cough. In an exemplary embodiment, the disease is associated with a Legionella species. In another exemplary embodiment, the disease is pneumonia or pontiac fever. In an exemplary embodiment, the disease is associated with a Francisella species. In another exemplary embodiment, the disease is tularemia. In an exemplary embodiment, the disease is associated with a Brucella species. In another exemplary embodiment, the disease is brucellosis. In an exemplary embodiment, the disease is associated with a Pasteurella species. In another exemplary embodiment, the disease is a skin infection. In an exemplary embodiment, the disease is associated with a Gardnerella species. In another exemplary embodiment, the disease is vaginitis. In an exemplary embodiment, the disease is associated with a Spirochetes species. In another exemplary embodiment, the disease is syphilis or Lyme disease. In an exemplary embodiment, the disease is associated with a Chlamydia species. In another exemplary embodiment, the disease is chlamydia. In an exemplary embodiment, the disease is associated with a Rickettsiae species. In another exemplary embodiment, the disease is Rocky Mountain spotted fever or typhus.

In an exemplary embodiment, the disease is associated with Mycoplasma pneumoniae. In another exemplary embodiment, the disease is tracheobronchitis or walking pneumonia. In an exemplary embodiment, the disease is associated with Ureaplasma urealyticum. In another exemplary embodiment, the disease is urethritis. In another exemplary embodiment, the disease is pyelonenephritis. In another exemplary embodiment, the disease is an intra-abdominal infection. In another exemplary embodiment, the disease is febrile neutropenia. In another exemplary embodiment, the disease is a pelvic infection. In another exemplary embodiment, the disease is bacteraemia. In another exemplary embodiment, the disease is septicaemia.

In any of the methods according to the invention set forth above, it is preferred that the aminoacyl tRNA synthetase is an aminoacyl tRNA synthetase comprising an editing domain. The editing domain is encoded by a portion of the aminoacyl tRNA synthetase involved in proofreading. The editing domain is preferably encoded by a DNA portion having at least conserved residues compared after alignment with the editing site of the leucyl-tRNA synthetase, valyl-tRNA synthetase and isoleucyl-tRNA synthetase. More preferably the synthetase is selected from the group consisting of the valyl-tRNA synthetase, isoleucyl-tRNA synthetase, leucyl-tRNA synthetase, alanyl-tRNA synthetase, prolyl-tRNA synthetase, threonyl-tRNA synthetase, phenyl-tRNA synthetase and lysyl-tRNA synthetase which are known to have an editing site or domain (see for Ile RS Baldwin, A. N. and Berg, P. (1966) J. Biol. Chem. 241, 839-845 and Eldred, E. W. and Schimmel, P. R. (1972) J. Biol. Chem. 247, 2961-2964; for Val RS, Fersht, A. R. and Kaethner, M. M. (1976) Biochemistry. 15 (15), 3342-3346; for Leu RS, English, S. et al., (1986) Nucleic Acids Research. 14 (19), 7529-7539; for Ala RS, Tsui, W. C. and Fersht, A. R. (1981) Nucleic Acids Research. 9, 7529-7539; for Pro RS, Beuning, P. J. and Musier-Forsyth, K. (2000) PNAS. 97 (16), 8916-8920; for Thr RS, Sankaranarayanan, R. et al., (2000) Nat. Struct. Biol. 7, 461-465 and Musier-Foryth, K. and Beuning, P. J. (2000) Nat. Struct. Biol. 7, 435-436; for PheRS, Yarus, M. (1972) PNAS. 69, 1915-1919 and for LysRS, Jakubowski, H. (1997) Biochemistry. 36, 11077-11085. In another exemplary embodiment, R* is H.

### IX. Pharmaceutical Formulations

In another aspect, the invention is a pharmaceutical formulation which includes: (a) a pharmaceutically acceptable excipient; and (b) a compound of the invention. In another aspect, the pharmaceutical formulation includes: (a) a pharmaceutically acceptable excipient; and (b) a compound according to a formula described herein. In another aspect, the pharmaceutical formulation includes: (a) a pharmaceutically acceptable excipient; and (b) a compound described herein, or a salt, prodrug, hydrate or solvate thereof, or a combination thereof. In another aspect, the pharmaceutical formulation includes: (a) a pharmaceutically acceptable excipient; and (b) a compound described herein, or a salt, hydrate or solvate thereof, or a combination thereof. In another aspect, the pharmaceutical formulation includes: (a) a pharmaceutically acceptable excipient; and (b) a compound described herein, or a salt, hydrate or solvate thereof. In another aspect, the pharmaceutical formulation includes: (a) a pharmaceutically acceptable excipient; and (b) a salt of a compound described herein. In an exemplary embodiment, the salt is a pharmaceutically acceptable salt. In another aspect, the pharmaceutical formulation includes: (a) a pharmaceutically acceptable excipient; and (b) a prodrug of a compound described herein. In another aspect, the pharmaceutical formulation includes: (a) a pharmaceutically acceptable excipient; and (b) a compound described herein. In an exemplary embodiment, the pharmaceutical formulation is a unit dosage form. In an exemplary embodiment, the pharmaceutical formulation is a single unit dosage form.

The pharmaceutical formulations of the invention can take a variety of forms adapted to the chosen route of administration. Those skilled in the art will recognize various synthetic methodologies that may be employed to prepare non-toxic pharmaceutical formulations incorporating the compounds described herein. Those skilled in the art will recognize a wide variety of non-toxic pharmaceutically acceptable solvents that may be used to prepare solvates of the compounds of the invention, such as water, ethanol, propylene glycol, mineral oil, vegetable oil and dimethylsulfoxide (DMSO).

The pharmaceutical formulation of the invention may be administered orally, topically, intraperitoneally, parenterally, by inhalation or spray or rectally in unit dosage forms containing conventional non-toxic pharmaceutically acceptable carriers, adjuvants and vehicles. It is further understood that the best method of administration may be a combination of methods. Oral administration in the form of a pill, capsule, elixir, syrup, lozenge, troche, or the like is particularly preferred. The term parenteral as used herein includes subcutaneous injections, intradermal, intravascular (e.g., intravenous), intramuscular, spinal, intrathecal injection or like injection or infusion techniques. In an exemplary embodiment, the pharmaceutical formulation is administered orally. In an exemplary embodiment, the pharmaceutical formulation is administered intravenously. In an exemplary embodiment, the pharmaceutical formulation is administered in a topically effective dose. In an exemplary embodiment, the pharmaceutical formulation is administered in a cosmetically effective dose. In an exemplary embodiment, the pharmaceutical formulation is administered in an orally effective dose.

The pharmaceutical formulations containing compounds of the invention are preferably in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known in the art for the manufacture of pharmaceutical formulations, and such compositions may contain one or more agents selected from the group consisting of sweetening agents, flavoring agents, coloring agents and preserving agents in order to provide pharmaceutically elegant and palatable preparations. Tablets may contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; and dispersing or wetting agents, which may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical formulations of the invention may also be in the form of oil-in-water emulsions and water-in-oil emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth; naturally-occurring phosphatides, for example soy bean, lecithin, and esters or partial esters derived from fatty acids and hexitol; anhydrides, for example sorbitan monooleate; and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical formulations may be in the form of a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition of the invention may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials are cocoa butter and polyethylene glycols.

Alternatively, the compositions can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For administration to non-human animals, the composition containing the therapeutic compound may be added to the animal's feed or drinking water. Also, it will be convenient to formulate animal feed and drinking water products so that the animal takes in an appropriate quantity of the compound in its diet. It will further be convenient to present the compound in a composition as a premix for addition to the feed or drinking water. The composition can also added as a food or drink supplement for humans.

Dosage levels of the order of from about 5 mg to about 250 mg per kilogram of body weight per day and more preferably from about 25 mg to about 150 mg per kilogram of body weight per day, are useful in the treatment of the above-indicated conditions. The amount of active ingredient that may be combined with the carrier materials to produce a unit dosage form will vary depending upon the condition being treated and the particular mode of administration. Unit dosage forms will generally contain between from about 1 mg to about 500 mg of an active ingredient.

Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy.

In an exemplary embodiment, the unit dosage form contains from about 1 mg to about 800 mg of a compound of the invention. In an exemplary embodiment, the unit dosage form contains from about 1 mg to about 500 mg of an active ingredient. In an exemplary embodiment, the unit dosage form contains from about 100 mg to about 800 mg of a compound of the invention. In an exemplary embodiment, the unit dosage form contains from about 200 mg to about 500 mg of a compound of the invention. In an exemplary embodiment, the unit dosage form contains from about 500 mg to about 800 mg of a compound of the invention. In an exemplary embodiment, the unit dosage form contains from about 1 mg to about 100 mg of a compound of the invention. In an exemplary embodiment, the unit dosage form contains from about 10 mg to about 100 mg of a compound of the invention. In an exemplary embodiment, the unit dosage form contains from about 50 mg to about 100 mg of a compound of the invention. In an exemplary embodiment, the unit dosage form contains from about 75 mg to about 200 mg of a compound of the invention. In an exemplary embodiment, the unit dosage form contains from about 1 mg to about 5 mg of a compound of the invention. In an exemplary embodiment, the unit dosage form contains from about 10 mg to about 25 mg of a compound of the invention. In an exemplary embodiment, the unit dosage form contains from about 50 mg to about 350 mg of a compound of the invention. In an exemplary embodiment, the unit dosage form contains from about 200 mg to about 400 mg of a compound of the invention.

In an exemplary embodiment, the daily dosage contains from about 1 mg to about 800 mg of a compound of the invention. In an exemplary embodiment, the daily dosage contains from about 1 mg to about 500 mg of an active ingredient. In an exemplary embodiment, the daily dosage contains from about 100 mg to about 800 mg of a compound of the invention. In an exemplary embodiment, the daily dosage contains from about 200 mg to about 500 mg of a compound of the invention. In an exemplary embodiment, the daily dosage contains from about 500 mg to about 800 mg of a compound of the invention. In an exemplary embodiment, the daily dosage contains from about 1 mg to about 100 mg of a compound of the invention. In an exemplary embodiment, the daily dosage contains from about 10 mg to about 100 mg of a compound of the invention. In an exemplary embodiment, the daily dosage contains from about 50 mg to about 100 mg of a compound of the invention. In an exemplary embodiment, the daily dosage contains from about 75 mg to about 200 mg of a compound of the invention. In an exemplary embodiment, the daily dosage contains from about 1 mg to about 5 mg of a compound of the invention. In an exemplary embodiment, the daily dosage contains from about 10 mg to about 25 mg of a compound of the invention. In an exemplary embodiment, the daily dosage contains from about 50 mg to about 350 mg of a compound of the invention. In an exemplary embodiment, the daily dosage contains from about 200 mg to about 400 mg of a compound of the invention.

Preferred compounds of the invention will have desirable pharmacological properties that include, but are not limited to, oral bioavailability, low toxicity, low serum protein binding and desirable in vitro and in vivo half-lives. Penetration of the blood brain barrier for compounds used to treat CNS disorders is necessary, while low brain levels of compounds used to treat peripheral disorders are often preferred.

Assays may be used to predict these desirable pharmacological properties. Assays used to predict bioavailability include transport across human intestinal cell monolayers, including Caco-2 cell monolayers. Toxicity to cultured hepatocycles may be used to predict compound toxicity. Penetration of the blood brain barrier of a compound in humans may be predicted from the brain levels of laboratory animals that receive the compound intravenously.

Serum protein binding may be predicted from albumin binding assays. Such assays are described in a review by Oravcova, et al. (Journal of Chromatography B (1996) volume 677, pages 1-27).

In vitro half-lives of compounds may be predicted from assays of microsomal half-life as described by Kuhnz and Gieschen (Drug Metabolism and Disposition, (1998) volume 26, pages 1120-1127).

The amount of the composition required for use in treatment will vary not only with the particular compound selected but also with the route of administration, the nature of the condition being treated and the age and condition of the patient and will ultimately be at the discretion of the attendant physician or clinician.

### IX.a) Testing

Preferred compounds for use in the pharmaceutical formulations described herein will have certain pharmacological properties. Such properties include, but are not limited to, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcova et al. (1996, J. Chromat. B677: 1-27). Compound half-life is inversely proportional to the frequency of dosage of a compound. In vitro half-lives of compounds may be predicted from assays of microsomal half-life as described by Kuhnz and Gleschen (Drug Metabolism and Disposition, (1998) volume 26, pages 1120-1127).

Toxicity and therapeutic efficacy of such compounds can be determined by standard pharmaceutical procedures in cell cultures or experimental animals, e.g., for determining the LD50 (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the unit dosage form employed and the route of administration utilized. The exact formulation, route of administration and dosage can be chosen by the individual physician in view of the patient's condition. (See, e.g. Fingl et al., 1975, in “The Pharmacological Basis of Therapeutics”, Ch. 1, p. 1).

### IX.b) Administration

For any compound used in the method of the invention, the therapeutically effective dose can be estimated initially from cell culture assays, as disclosed herein. For example, a dose can be formulated in animal models to achieve a circulating concentration range that includes the EC₅₀ (effective dose for 50% increase) as determined in cell culture, i.e., the concentration of the test compound which achieves a half-maximal inhibition of bacterial cell growth. Such information can be used to more accurately determine useful doses in humans.

In general, the compounds prepared by the methods, and from the intermediates, described herein will be administered in a therapeutically or cosmetically effective amount by any of the accepted modes of administration for agents that serve similar utilities. It will be understood, however, that the specific dose level for any particular patient will depend upon a variety of factors including the activity of the specific compound employed, the age, body weight, general health, sex, diet, time of administration, route of administration, and rate of excretion, drug combination, the severity of the particular disease undergoing therapy and the judgment of the prescribing physician. The drug can be administered from once or twice a day, or up to 3 or 4 times a day.

Dosage amount and interval can be adjusted individually to provide plasma levels of the active moiety that are sufficient to maintain bacterial cell growth inhibitory effects. Usual patient dosages for systemic administration range from 0.1 to 1000 mg/day, preferably, 1-500 mg/day, more preferably 10-200 mg/day, even more preferably 100-200 mg/day. Stated in terms of patient body surface areas, usual dosages range from 50-91 mg/m²/day.

The amount of the compound in a formulation can vary within the full range employed by those skilled in the art. Typically, the formulation will contain, on a weight percent (wt %) basis, from about 0.01-10 wt % of the drug based on the total formulation, with the balance being one or more suitable pharmaceutical excipients. Preferably, the compound is present at a level of about 0.1-3.0 wt %, more preferably, about 1.0 wt %.

Exemplary embodiments are summarized herein below.

In an exemplary embodiment, the invention provides a compound having a structure which is:

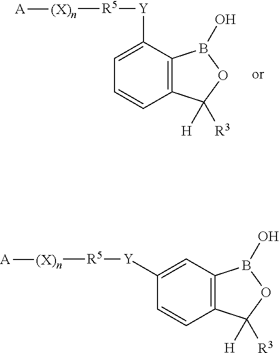

wherein Y is O or S; R⁵ is selected from the group consisting of substituted or unsubstituted alkylene and substituted or unsubstituted heteroalkylene; R³ is selected from the group consisting of H, cyano, substituted or unsubstituted nitroalkyl and substituted or unsubstituted aminoalkyl; A is an antibiotic or its pharmacophore; n is 0 or 1 or 2 or 3 or 4 or 5; each X are each the same or different and each are selected from the group consisting of: —(C₁ or C₂ or C₃ or C₄ or C₅ or C₆)alkylene, —(C₁ or C₂ or C₃ or C₄ or C₅ or C₆)alkenylene, —(C₁ or C₂ or C₃ or C₄ or C₅ or C₆)alkynylene, —(C₃ or C₄ or C₅ or C₆ or C₇ or C₈)cycloalkylene, —O—R¹³)—, wherein R¹³ represents hydrogen, (C₁ or C₂ or C₃ or C₄ or C₅ or C₆)alkyl, substituted (C₁ or C₂ or C₃ or C₄ or C₅ or C₆)alkyl, —S(O)ₘ—, wherein m is 0, 1, or 2, —N(R¹⁴)—, wherein R¹⁴ represents hydrogen, (C₁ or C₂ or C₃ or C₄ or C₅ or C₆)alkyl, substituted (C₁ or C₂ or C₃ or C₄ or C₅ or C₆)alkyl; arylene, heteroarylene, and bivalent heterocyclic structure containing 1 or 2 or 3 heteroatoms, wherein, the carbon or nitrogen atoms of X can be optionally substituted by 1 or 2 or 3 substituents, each of which is selected from the group consisting of (C₁ or C₂ or C₃ or C₄ or C₅ or C₆)alkyl, substituted (C₁ or C₂ or C₃ or C₄ or C₅ or C₆)alkyl, heterocycloalkyl, amino, (C₁ or C₂ or C₃ or C₄ or C₅ or C₆)alkylamino, di(C₁ or C₂ or C₃ or C₄ or C₅ or C₆)alkylamino, hydroxyl, and (C₁ or C₂ or C₃ or C₄ or C₅ or C₆)alkoxy; or a salt thereof.

In an exemplary embodiment, according to the above paragraph, A comprises a moiety which is a quinolone or an oxazolidinone.

In an exemplary embodiment, according to any of the above paragraphs, A comprises an oxazolidinone, and the oxazolidinone is selected from the group consisting of linezolid, torezolid, posizolid, ranbezolid, radezolid and eperezolid.

In an exemplary embodiment, according to any of the above paragraphs, the compound has a structure which is:

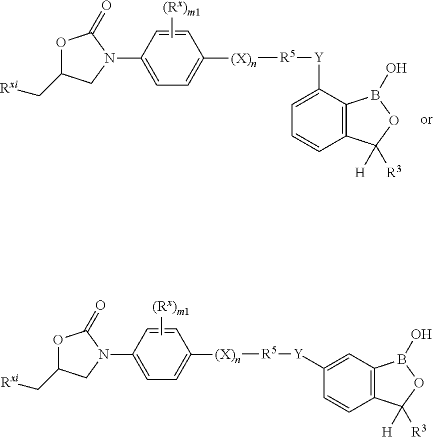

wherein R^{x} is halogen; R^{xi} is selected from the group consisting of CH₃CONH—, OH and substituted or unsubstituted triazole; m1 is 0 or 1 or 2.

In an exemplary embodiment, according to any of the above paragraphs, the compound has a structure which is:

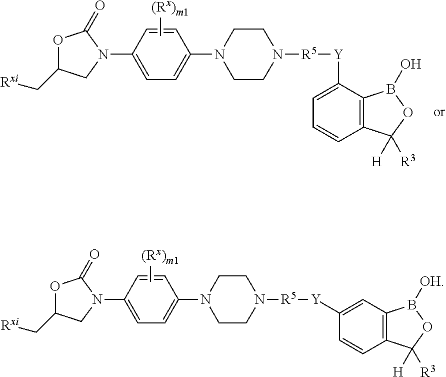

In an exemplary embodiment, according to any of the above paragraphs, A is a quinolone, and the quinolone is fluoroquinolone or non-fluoroquinolone.

In an exemplary embodiment, according to any of the above paragraphs, A is a quinolone, which is selected from the group consisting of nalidixic acid, oxolinic acid, piromidic acid, pipemidic acid, cinoxacin and flumequine.

In an exemplary embodiment, according to any of the above paragraphs, A is a quinolone, which is selected from the group consisting of ciprofloxacin, enoxacin, fleroxacin, lomefloxacin, nadifloxacin, norfloxacin, ofloxacin, pefloxacin and rufloxacin.

In an exemplary embodiment, according to any of the above paragraphs, A is a quinolone, which is selected from the group consisting of balofloxacin, gatifloxacin, grepafloxacin, levofloxacin, moxifloxacin, pazufloxacin, sparfloxacin, temafloxacin and tosufloxacin.

In an exemplary embodiment, according to any of the above paragraphs, A is a quinolone, which is selected from the group consisting of clinafloxacin, besifloxacin, gemifloxacin, sitafloxacin, alatrofloxacin, trovafloxacin and prulifloxacin.

In an exemplary embodiment, according to any of the above paragraphs, A is a quinolone, which is garenoxacin or delafloxacin.

In an exemplary embodiment, according to any of the above paragraphs, A is a quinolone, which is selected from the group consisting of danofloxacin, difloxacin, enrofloxacin, ibafloxacin, marbofloxacin, rosoxacin, orbifloxacin and sarafloxacin.

In an exemplary embodiment, according to any of the above paragraphs, the compound has a structure which is selected from the group consisting of:

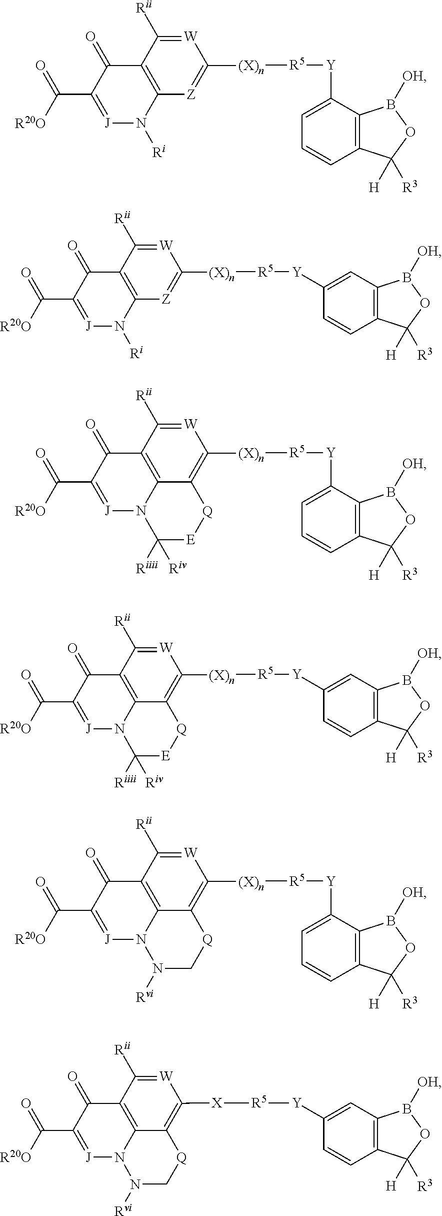

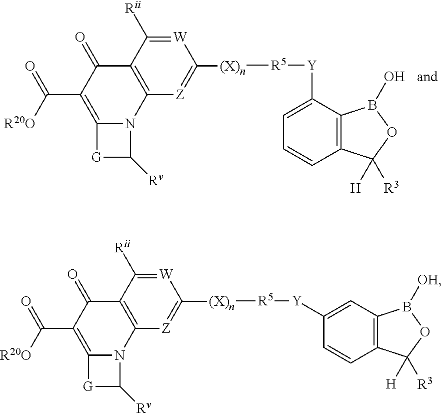

wherein R²⁰ is selected from the group consisting of H, unsubstituted alkyl, benzyl or a negative charge; Rⁱ is (C₁ or C₂ or C₃ or C₄ or C₅ or C₆)alkyl, halosubstituted (C₁ or C₂ or C₃ or C₄ or C₅ or C₆)alkyl, (C₃ or C₄ or C₅ or C₆)cycloalkyl, halosubstituted (C₃ or C₄ or C₅ or C₆)cycloalkyl, aryl, substituted aryl, heteroaryl, and halo-substituted heteroaryl; Rⁱⁱ is selected from the group consisting of hydrogen, halogen, amino and unsubstituted alkyl; and R^{iv} are each independently hydrogen or (C₁ or C₂ or C₃ or C₄ or C₅ or C₆)alkyl; W is selected from the group consisting of CH, CF, and N; Z is selected from the group consisting of N, CN, CH, C—F, C—Cl, and CR^{vii}, wherein R^{vii} is selected from the group consisting of unsubstituted alkyl, halosubstituted alkyl, unsubstituted alkoxy and halosubstituted alkoxy; Q is selected from the group consisting of CH₂, O and S; E is selected from the group consisting of CH₂, O and S; G is O or S; R^{v} is unsubstituted alkyl; R^{vi} is unsubstituted alkyl; and J is —CH or NH.

In an exemplary embodiment, according to any of the above paragraphs, R³ is selected from the group consisting of H, —CH₂NH₂ and —CH₂NO₂.

In an exemplary embodiment, according to any of the above paragraphs, R³ is —CH₂NH₂.

In an exemplary embodiment, according to any of the above paragraphs, the compound has a structure which is selected from the group consisting of:

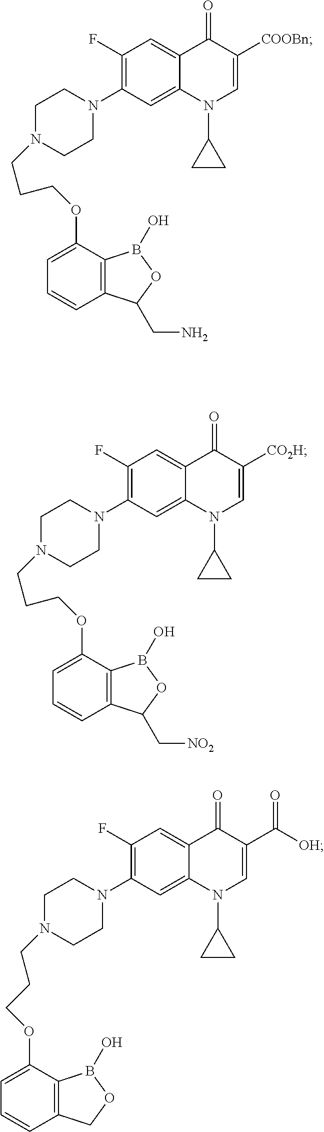

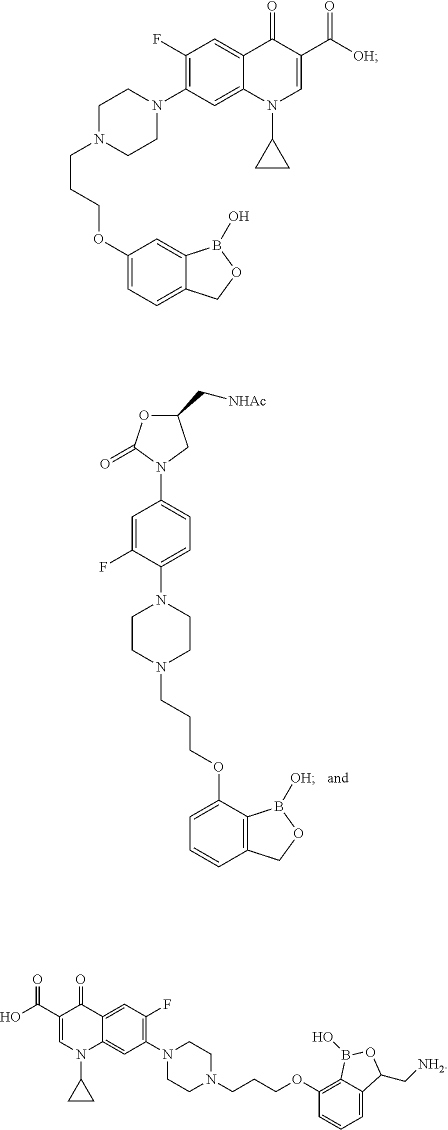

In an exemplary embodiment, according to any of the above paragraphs, the compound has a structure which is

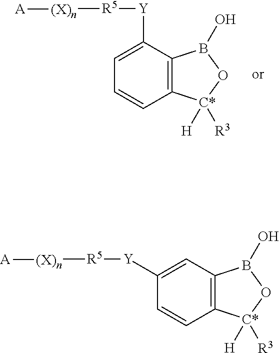

wherein C* is a carbon atom, with the proviso that when R³ is not H, C* is a stereocenter which has a configuration which is (R) or (S).

In an exemplary embodiment, according to any of the above paragraphs, there is the proviso that when R³ is not H, C* is a stereocenter which has a configuration which is (S).

In an exemplary embodiment, the invention provides a composition comprising: a) a first stereoisomer of the compound according to any of the above paragraphs; and b) at least one additional stereoisomer of the first stereoisomer; wherein the first stereoisomer is present in an enantiomeric excess of at least 80% relative to said at least one additional stereoisomer.

In an exemplary embodiment, according to any of the above paragraphs, the enantiomeric excess is at least 92%.

In an exemplary embodiment, according to any of the above paragraphs, the C* stereocenter of the first stereoisomer is in a (S) configuration.

In an exemplary embodiment, according to any of the above paragraphs, R³ is —CH₂NH₂.

In an exemplary embodiment, the invention provides a composition comprising: a compound according to any of the above paragraphs, wherein R³ is not H and the C* stereocenter is in a (S) configuration, and said composition is substantially free of a compound wherein the C* stereocenter is in a (R) configuration.

In an exemplary embodiment, according to any of the above paragraphs, the composition is substantially free of the enantiomer of the compound.

In an exemplary embodiment, the invention provides a combination comprising a compound according to any of the above paragraphs, or a pharmaceutically acceptable salt thereof, together with at least one other therapeutically active agent.

In an exemplary embodiment, the invention provides a pharmaceutical formulation comprising: a) a compound according to any of the above paragraphs, or a pharmaceutically acceptable salt thereof; and b) a pharmaceutically acceptable excipient.

In an exemplary embodiment, according to any of the above paragraphs, the pharmaceutical formulation is a unit dosage form.

In an exemplary embodiment, the invention provides a method of inhibiting an enzyme, comprising: contacting the enzyme with the compound according to any of the above paragraphs, thereby inhibiting the enzyme:

In an exemplary embodiment, according to any of the above paragraphs, the enzyme is a t-RNA synthetase which comprises an editing domain.

In an exemplary embodiment, according to any of the above paragraphs, the enzyme is a leucyl t-RNA synthetase.

In an exemplary embodiment, the invention provides a method of killing and/or preventing the growth of a microorganism, comprising: contacting the microorganism with an effective amount of a compound according to any of the above paragraphs, thereby killing and/or preventing the growth of the microorganism.

In an exemplary embodiment, according to any of the above paragraphs, the microorganism is a bacterium.

In an exemplary embodiment, the invention provides a method of treating and/or preventing a disease in an animal, comprising: administering to the animal a therapeutically effective amount of compound according to any of the above paragraphs, or a pharmaceutically-acceptable salt thereof, thereby treating and/or preventing the disease.

In an exemplary embodiment, according to any of the above paragraphs, the disease is caused by, or is mediated by, or involves a bacteria.

In an exemplary embodiment, according to any of the above paragraphs, the disease is caused by, or is mediated by, or involves a drug resistant bacterium.

In an exemplary embodiment, according to any of the above paragraphs, the disease is pneumonia.

In an exemplary embodiment, according to any of the above paragraphs, the animal is a human.

In an exemplary embodiment, according to any of the above paragraphs, the salt is a pharmaceutically acceptable salt.

The invention is further illustrated by the Examples that follow. The Examples are not intended to define or limit the scope of the invention.

### EXAMPLES

All solvents used were commercially available and were used without further purification. Reactions were typically run using anhydrous solvents under an inert atmosphere of N₂.

¹H, ¹³C, and ¹⁹F NMR spectra were recorded at 400 MHz for proton, 100 MHz for carbon-13, and 376 MHz for fluorine-19 on a Varian 300 MercuryPlus station with an Oxford AS400 Spectrometer equipped with a Varian 400 ATB PFG probe. All deuterated solvents typically contained 0.03% to 0.05% v/v tetramethylsilane, which was used as the reference signal (set at δ 0.00 for both ¹H and ¹³C).

Compounds are named using ChemDraw 7.0 or their catalogue name if commercially available.

Mass spectra were recorded on a Waters MS consisting of an Alliance 2795 (LC) and Waters Micromass ZQ detector at 120° C. The mass spectrometer was equipped with an electrospray ion source (ESI) operated in a positive or negative mode. The mass spectrometer was scanned between m/z=100-1000 with a scan time of 0.3 s.

Elemental Analysis for C, H and N composition was performed using a Costech Instrument Elemental Combustion System ECS4010 with a helium flow of 100 mL/min (14 psi), oxygen 20 mL/min (10 psi), air 25 psi and purge of 50 mL/min. The reported analyses are an average of two runs.

HPLC analyses were performed on a Water 600 Controller system with a Waters 717 Plus Autosampler and a Waters 2996 Photodiode Array Detector. The column used was an ACE C₁₈, 5 μm, 4.6×150 mm. A linear gradient was applied, starting at 95% A (A: 0.1% H₃PO₄ in water) and ending at 90% B (B: MeCN) over 6 min and then maintained at 90% B until the 10 min mark. The column was then re-equilibrated over 3 min to 95:5 with a total run time of 20 min. The column temperature was at rt with the flow rate of 1.0 mL/min. The Diode Array Detector was scanned from 200-400 nm. For high purity samples requiring baseline subtraction, a linear gradient was applied, starting at 99% A (A: 0.1% H₃PO₄ in water) and ending at 90% B (B: MeCN) over 15 min. The column was then re-equilibrated over 3 min to 99% A with a total run time of 23 min. The column temperature was at rt with the flow rate of 1.0 mL/min. The Diode Array Detector was scanned from 200-400 nm. A blank MeOH sample was run immediately prior to the sample of which purity was to be determined: this was then subtracted to obtain the baseline subtracted chromatogram.

Thin layer chromatography (TLC) was performed on Alugram® (Silica gel 60 F₂₅₄) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), vanillin (generated by dissolving about 1 g vanillin in 100 mL 10% H₂SO₄), potassium permanganate (generated by dissolving 1.5 g KMnO₄ and 10 g K₂CO₃ in 1.25 mL NaOH and 200 mL H₂O), ninhydrin (available commercially from Aldrich), or Magic Stain (generated by thoroughly mixing 25 g (NH₄)₆Mo₇O₂₄.4H₂O, 5 g (NH₄)₂Ce(IV)(NO₃)₆ in 450 mL H₂O and 50 mL conc H₂SO₄) to visualize the compound. Flash chromatography was preformed using typically 40-63 μm (230-400 mesh) silica gel from Silicycle following analogous techniques to those disclosed by Still et al. Typical solvents used for flash chromatography or thin layer chromatography (TLC) were mixtures of CHCl₃/MeOH, CH₂Cl₂/MeOH, EtOAc/MeOH and hexane/EtOAc. Reverse phase flash chromatography were performed on a Biotage® using a Biotage C₁₈ cartridges and a H₂O/MeOH gradient (typically eluting from 5% MeOH/H₂O to 90% MeOH/H₂O).

Preparative chromatography was performed on either a Waters Prep LC 4000 System using a Waters 2487 Diode Array or on a Waters LC Module 1 plus. The column used were either a Waters×Terra Prep C₁₈, 5 μm, 30×100 mm, Phenomenex Luna C₁₈, 5 μm, 21.6×250 mm, or a Phenomenex Gemini C₁₈, 5 μm, 100×30 mm. Narrow gradients with MeCN/H₂O (water containing either 0.1% TFA, 0.1% AcOH, 0.1% HCO₂H or 0.1% NH₄OAc) were used to elute the compound at a flow rate of approximately 20 mL/min and a total run time between 20-30 min.

For enantiomeric excess determination, chiral HPLC analysis was performed on a Waters 600 Controller and Multisolvent Delivery System using a Waters 717+ Autosampler and a Waters 996 Photodiode Array Detector with a Crownpak CR(+) column, eluting with 85:15 pH 1 perchloric acid in H₂O/MeOH mobile phase. The pH 1 perchloric acid was generated by adding 16.3 g of 70% perchloric acid to 1 L of distilled H₂O.

Starting materials used were either available from commercial sources or prepared according to literature procedures and had experimental data in accordance with those reported. 6-aminobenzo[c][1,2]oxaborol-1(3H)-ol, for example, can be synthesized according to the methods described in U.S. patent application Ser. No. 12/142,692, as well as U.S. Pat. Pubs. US20060234981 and US20070155699.

### Example 1

Benzyl 1-cyclopropyl-6-fluoro-7-(4-(3-(1-hydroxy-3-(nitromethyl)-1,3-dihydrobenzo[c][1,2]oxaborol-7-yloxy)propyl)piperazin-1-yl)-4-oxo-1,4-dihydroquinoline-3-carboxylate (F1)

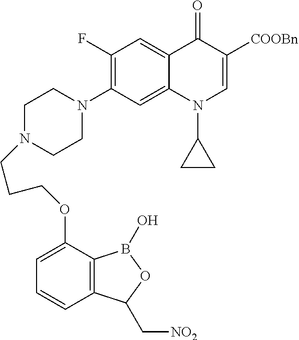

### Benzyl 1-cyclopropyl-6-fluoro-4-oxo-7-(piperazin-1-yl)-1,4-dihydroquinoline-3-carboxylate

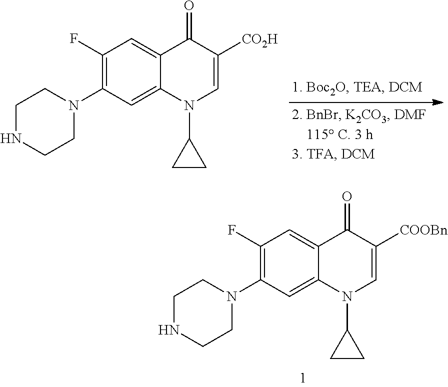

To ciprofloxacin (2.5 g, 6.8 mmol) (purchased from TCI America (Portland, Oreg.)), TEA (2.78 mL, 20.4 mmol) was added Boc₂O (1.78 g, 8.15 mmol). the reaction was stirred at r.t. for 5 h and worked up with EtOAc, H₂O, brine and dried over Na₂SO₄, The crude was added BnBr (0.79 mL, 6.62 mmol), K₂CO₃ (2.6 g, 20 mmol) and DMF (15 mL) and heated to 115° C. for 2 h. After cooled down, the mixture was extracted with EtOAc and H₂O followed by brine, dried over Na₂SO₄. Flash chromatography was applied to purify the intermediate, half of the resulting product was treated with TFA (20 mL, 25% in DCM) to give compound (1). ¹H NMR (400 MHz, DMSO-d₆) δ (ppm): TFA salt: 8.83 (s, 2H), 8.50 (s, 1H), 7.84 (d, J=13 Hz, 1H), 7.49 (m, 3H), 7.39 (m, 2H), 7.34 (m, 1H), 5.27 (s, 2H), 3.68 (m, 1H), 3.44 (m, 4H), 3.33 (m, 4H), 1.26 (m, 2H), 1.11 (m, 2H); MS (ESI) m/z=422.1 (M+H, Positive), HPLC: 2.82 min.

### 2-Bromo-3-(3-bromopropoxy)benzaldehyde

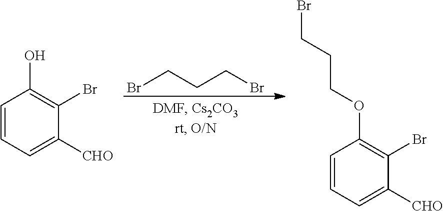

2-Bromo-3-hydroxybenzaldehyde (2 g, 10 mmol), 1,3 dibromopropane (1.2 mL, 10 mmol), Cs₂CO₃ (5 g, 30 mmol), and DMF (10 mL). The reaction was stirred at r.t. overnight, worked up with EtOAc, H₂O, brine, dried over Na₂SO₄, Purification: flash chromatography; column (5%-20% EtOAc/hexane) gave the title compound as white solid (yield 1.7 g 53). ¹H NMR (400 MHz, CDCl₃) δ (ppm): 10.43 (s, 1H), 7.54 (dd, J=1.7, 8.0 Hz, 1H), 7.37 (t, J=8.0 Hz, 1H), 7.15 (dd, J=8.0, 1.7 Hz, 1H), 4.22 (t, J=5.6 Hz, 2H), 3.71 (t, J=6.3 Hz, 2H), 2.41 (m, 2H); HPLC: 3.20 min.

### 3-(3-Bromopropoxy)-2-(4,4,5,5-tetramethyl-1,3,2-dioxaborolan-2-yl)benzaldehyde

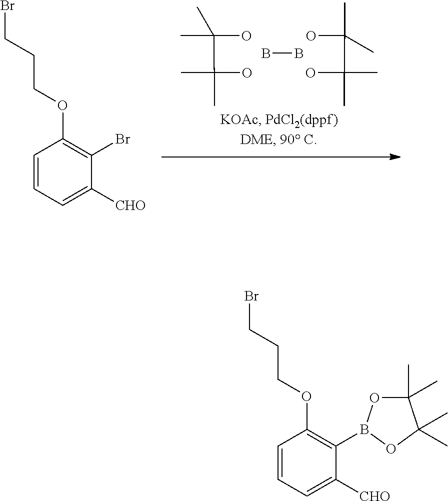

2-Bromo-3-(3-bromopropoxy)benzaldehyde (0.8 g, 2.48 mmol), B₂Pin₂ (0.82 g, 3.22 mmol), KOAc (0.75 g, 7.45 mmol), Pd(dppf)Cl₂ (80 mg, 0.074 mmol) and DME (15 mL). The reaction was heated at 80° C. and refluxed overnight, filtered and washed with EtOAc, Purification: flash chromatography; column (10%-30% EtOAc/hexane) gave the title compound (yield 0.2 g 22%). ¹H NMR (400 MHz, CDCl₃) δ (ppm): 9.43 (s, 1H), 7.48 (t, J=8.0 Hz, 1H), 7.41 (d, J=7.8 Hz, 1H), 7.09 (d, J=8.0 Hz, 1H), 4.15 (t, J=5.7 Hz, 2H), 3.61 (t, J=6.6 Hz, 2H), 2.31 (m, 2H), 1.45 (s, 9H); MS (ESI) m/z=345.1 (Boronic acid+OAc, negative); HPLC: 1.99 min(Boronic acid).

### 7-(3-Bromopropoxy)-3-(nitromethyl)benzo[c][1,2]oxaborol-1(3H)-ol

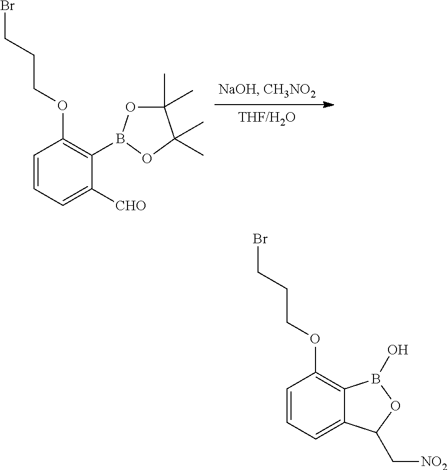

To a mixture of NaOH (1M in H₂O, 1.35 mL) and nitromethane (0.108 mL, 2.03 mmol) in THF/H₂O was added 3-(3-bromopropoxy)-2-(4,4,5,5-tetramethyl-1,3,2-dioxaborolan-2-yl)benzaldehyde (0.25 g, 0.67 mmol), the reaction was stirred at r.t. for 6 h then added 1N HCl (2 mL) and stirred overnight. The reaction was worked up with EtOAc, H₂O, brine and dried over Na₂SO₄, Purification: flash chromatography; column (20%-50% EtOAc/hexane) gave the title compound (yield 0.15 g 67%). ¹H NMR (400 MHz, CDCl₃) δ (ppm): 7.49 (t, J=7.8 Hz, 1H), 6.92 (d, J=7.6 Hz, 1H), 6.87 (d, J=8.0 Hz, 1H), 5.85 (dd, J=9.0, 3.5 Hz, 1H), 5.11 (s, 1H), 4.74 (dd, J=13.3, 3.5 Hz, 1H), 4.45 (dd, J=13.1, 9.0 Hz, 1H), 4.24 (t, J=5.9 Hz, 2H), 3.61 (t, J=6.2 Hz, 2H), 2.38 (m, 2H); HPLC: 3.40 min.

### Benzyl 1-cyclopropyl-6-fluoro-7-(4-(3-(1-hydroxy-3-(nitromethyl)-1,3-dihydrobenzo[c][1,2]oxaborol-7-yloxy)propyl)piperazin-1-yl)-4-oxo-1,4-dihydroquinoline-3-carboxylate

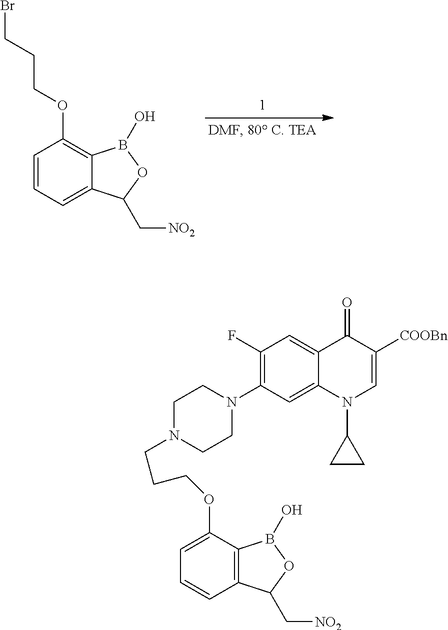

7-(3-Bromopropoxy)-3-(nitromethyl)benzo[c][1,2]oxaborol-1(3H)-ol (0.05 g, 0.15 mmol), benzyl 1-cyclopropyl-6-fluoro-4-oxo-7-(piperazin-1-yl)-1,4-dihydroquinoline-3-carboxylate (0.064 g, 0.15 mmol), TEA (0.062 mL, 0.45 mmol) and DMF (3 mL). The reaction was heated at 80° C. for 3 h, remove most solvent, Purification: preparative HPLC gave the title compound (yield 0.025 g, 25%); MS (ESI) m/z=671.2 (M+1, positive); HPLC: 3.63 min.

### Benzyl 7-(4-(3-(3-(aminomethyl)-1-hydroxy-1,3-dihydrobenzo[c][1,2]oxaborol-7-yloxy)propyl)piperazin-1-yl)-1-cyclopropyl-6-fluoro-4-oxo-1,4-dihydroquinoline-3-carboxylate (F2)

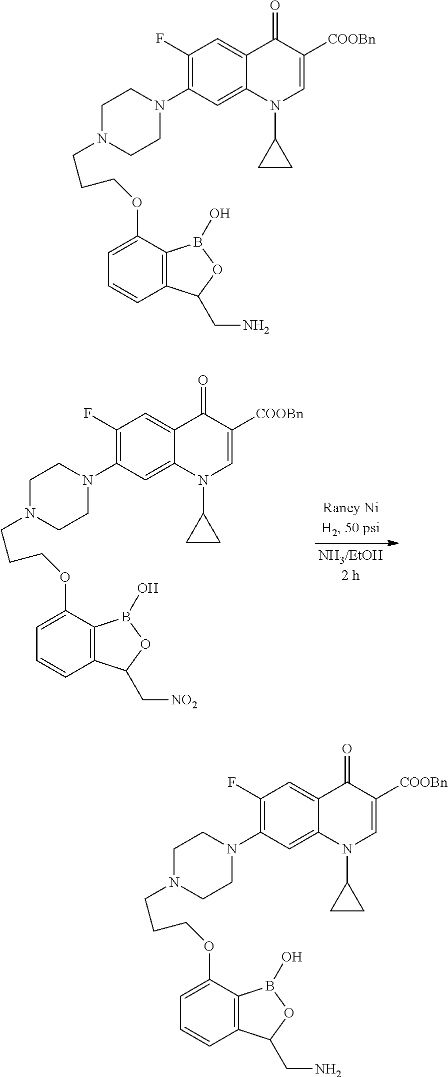

Benzyl 1-cyclopropyl-6-fluoro-7-(4-(3-(1-hydroxy-3-(nitromethyl)-1,3-dihydrobenzo[c][1,2]oxaborol-7-yloxy)propyl)piperazin-1-yl)-4-oxo-1,4-dihydroquinoline-3-carboxylate (25 mg, 0.038 mmol), Raney Ni (30 mg), and 2 M NH₃ in EtOH (3 mL) was hydrogenated at 50 psi at rt for 5 h in Parr Shaker. After filtration, the filtrate was concentrated in vacuo. The residue was purified by preparative HPLC, then lyophilized to give the title compound. (yield: 2.8 mg 11%). ¹H NMR (400 MHz, DMSO-d₆) δ (ppm): TFA salt: 9.73 (bs, 1H), 8.94 (s, 1H), 8.51 (s, 1H), 7.98 (s, 3H), 7.86 (d, J=11 Hz, 1H), 7.49 (m, 4H), 7.40 (t, J=7.6, 2H), 7.33 (t, J=7.2 Hz, 1H), 7.11 (d, J=6.4 Hz, 1H), 6.95 (d, J=8.4 Hz, 1H), 5.27 (m, 3H), 4.16 (m, 2H), 3.84 (m, 1H), 3.70 (m, 3H), 3.40 (m, 4H), 3.33 (m, 4H), 2.82 (m, 1H), 2.54 (m, 1H), 2.32 (m, 2H), 1.26 (m, 2H), 1.11 (m, 2H); MS (ESI) m/z=641.2 (M+1, positive); HPLC purity: 95% (220 nm), 95 (254 nm) 3.18 min.

### 1-Cyclopropyl-6-fluoro-7-(4-(3-(1-hydroxy-3-(nitromethyl)-1,3-dihydrobenzo[c][1,2]oxaborol-7-yloxy)propyl)piperazin-1-yl)-4-oxo-1,4-dihydroquinoline-3-carboxylic acid (F3)

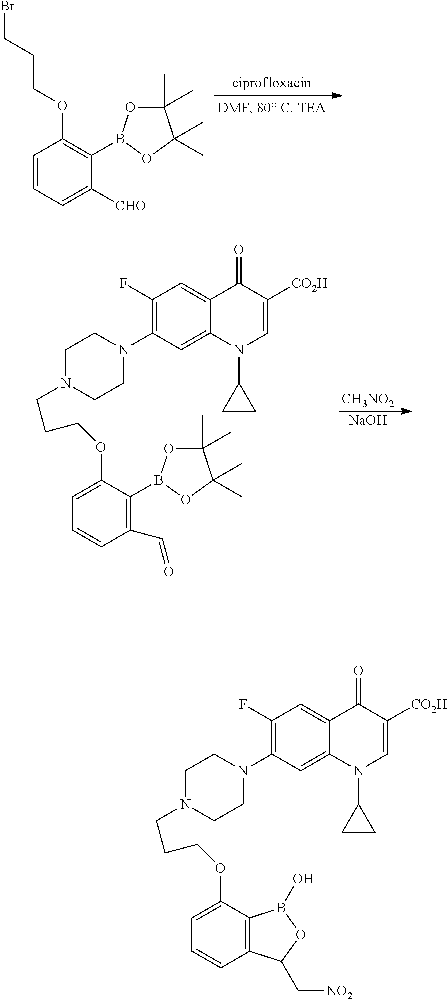

3-(3-bromopropoxy)-2-(4,4,5,5-tetramethyl-1,3,2-dioxaborolan-2-yl)benzaldehyde (0.03 g, 0.08 mmol), ciprofloxacin (0.05 g, 0.15 mmol), TEA (0.5 mL) and DMF (2 mL). The reaction was heated at 80° C. for 6 h, remove most solvent, Purification: preparative HPLC gave 1-cyclopropyl-6-fluoro-7-(4-(3-(3-formyl-2-(4,4,5,5-tetramethyl-1,3,2-dioxaborolan-2-yl)phenoxy)propyl)piperazin-1-yl)-4-oxo-1,4-dihydroquinoline-3-carboxylic acid (yield 0.014 g, 28%); MS (ESI) m/z=538.1 (Boronic acid+H, positive); HPLC: 2.73 min. To a mixture of NaOH (1M in H₂O, 0.5 mL) and Nitromethane (0.028 mL, 0.5 mmol) in THF/H₂O was added 1-cyclopropyl-6-fluoro-7-(4-(3-(3-formyl-2-(4,4,5,5-tetramethyl-1,3,2-dioxaborolan-2-yl)phenoxy)propyl)piperazin-1-yl)-4-oxo-1,4-dihydroquinoline-3-carboxylic acid (0.09 g, 0.17 mmol), the reaction was stirred at r.t. for 6 h then added 1N HCl (2 mL) and stirred overnight. The solvent was removed and the crude mixture was submitted to preparative HPLC for purification and gave the title compound (F3). (yield 0.012 g 14%). ¹H NMR (400 MHz, DMSO-d₆) δ (ppm): TFA salt: 9.63 (bs, 1H), 9.22 (s, 1H), 8.71 (s, 1H), 7.99 (d, J=13 Hz, 1H), 7.62 (d, J=7 Hz, 1H), 7.51 (t, J=7.8, 1H), 7.14 (d, J=7.8 Hz, 1H), 6.95 (d, J=8 Hz, 1H)), 5.74 (dd, J=9.0, 2.7 Hz, 1H), 5.34 (dd, J=13.5, 2.8 Hz, 1H), 4.57 (dd, J=13.3, 9 Hz, 1H), 4.18 (t, J=6 Hz, 2H), 3.90 (m, 2H), 3.86 (m, 2H), 3.73 (m, 2H), 3.33 (m, 4H), 2.22 (m, 2H), 1.32 (m, 2H), 1.22 (m, 2H); MS (ESI) m/z=581.2 (M+H, positive); HPLC purity: 99% (220 nm), 99 (254 nm) 3.41 min.

### 1-Cyclopropyl-6-fluoro-7-{4-[3-(1-hydroxy-1,3-dihydro-benzok[c][1,2]oxaborol-7-yloxy)-propyl]-piperazin-1-yl}-4-oxo-1,4-dihydro-quinoline-3-carboxylic acid; formate salt (F4)

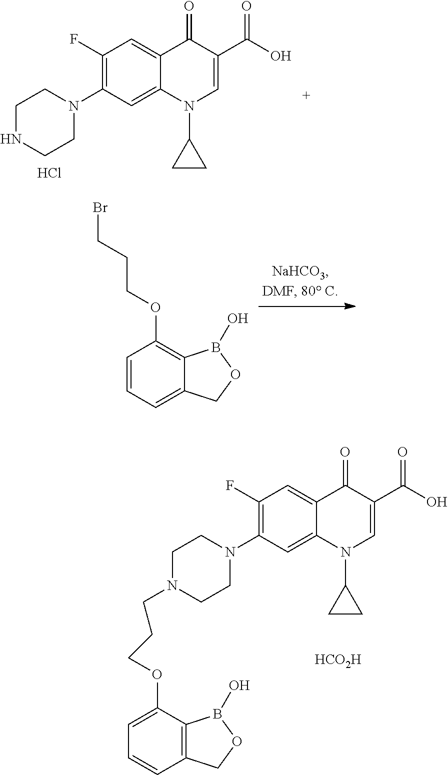

To a stirred solution of 7-(3-bromo-propoxy)-3H-benzo[c][1, 2]oxaborol-1-ol (0.12 g, 0.44 mmol) in anhydrous DMF (4 mL), 1-cyclopropyl-6-fluoro-4-oxo-7-piperazin-1-yl-1,4-dihydro-quinoline-3-carboxylic acid HCl (0.12 g, 0.33 mmol), (purchased from TCI America (Portland, Oreg.)) and powdered NaHCO₃ (0.075 g, 0.89 mmol) were added, and the mixture was stirred at 80° C. overnight. The solids were removed by filtration and the filtrate was concentrated in vacuo providing a light yellow viscous product (0.18 g). Purification by preparative HPLC (acetonitrile/water with 0.1% formic acid) generated 0.013 g (6%) of the title compound (F4) as light yellow solid. ¹H NMR (400 MHz, DMSO-d₆) δ ppm 8.64 (s, 1H), 8.20 (s, 1H, formic acid), 7.89 (d, J=13.2 Hz, 1H), 7.55 (d, J=7.2 Hz, 1H), 7.39 (t, J=8.0 Hz, 1H), 6.92 (d, J=7.6 Hz, 1H), 6.81 (d, J=8.0 Hz, 1H), 4.90 (s, 2H), 4.09 (t, J=6.0 Hz, 2H), 3.80 (br. s., 1H), 3.34 (s, 4H), 2.60 (s, 4H), 2.52 (t, J=7.2 Hz, 2H), 1.96-1.88 (m, 2H), 1.30-1.27 (m, 2H), 1.21-1.13 (m, 2H); MS (ESI): m/z=522 (M+1, positive); HPLC purity: 94.56% (MaxPlot 200-400 nm), 95.72% (220 nm).

### 1-Cyclopropyl-6-fluoro-7-(4-(3-(1-hydroxy-1,3-dihydrobenzo[c][1,2]oxaborol-6-yloxy)propyl)piperazin-1-yl)-4-oxo-1,4-dihydroquinoline-3-carboxylic acid (F5)

### 2-bromo-4-hydroxybenzaldehyde

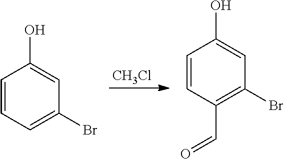

The mixture of aqueous KOH (30%, 160 ml) and 3-bromophenol (10.04 g, 58 mmol) was heated to 70-75° C. and then ethanol (5 ml) was added in one portion followed by dropwise addition of chloroform (28 ml, 0.35 mol) for 1 h at 75° C. The reaction mixture was stirred for additional 3 h at that temperature. After cooling to room temperature and acidification with 10N HCl, most of the by-products were removed by steam distillation. The residue was cooled and the precipitate was separated by filtration. Column chromatography of the crude solid on silica gel with a mixture of hexane/ethyl acetate (2:1, v/v) as eluent afforded 1.75 g (15%) of 2-bromo-4-hydroxybenzaldehyde

### 2-bromo-4-(3-bromopropoxy)benzaldehyde

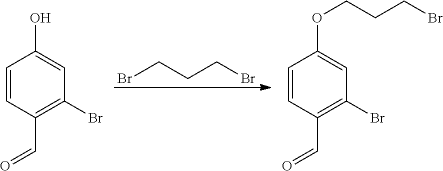

A mixture of 2-bromo-4-hydroxybenzaldehyde (12 g, 60 mmol), Cs₂CO₃ (29.25 g, 90 mmol) and 1,3-dibromopropane (60.6 g, 300 mmol) in MeCN (300 ml) was refluxed for 10 min with stirring. The reaction mixture was filtered and concentrated. The residue was dissolved in ethyl acetate (500 ml), washed with brine, dried over Na₂SO₄, filtered, concentrated and purified by silica gel column chromatography to afford 16.2 g of 2-bromo-4-(3-bromopropoxy)benzaldehyde as a white solid. Yield 84%.

### 4-(3-bromopropoxy)-2-(4,4,5,5-tetramethyl-1,3,2-dioxaborolan-2-yl)benzaldehyde

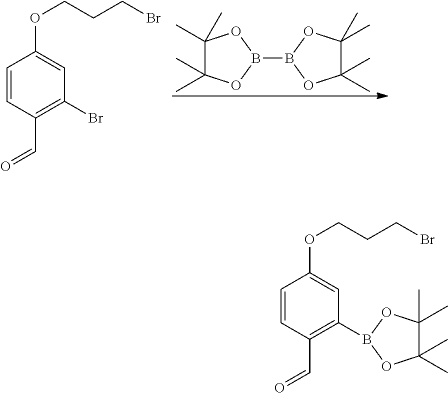

A mixture of 2-bromo-4-(3-bromopropoxy)benzaldehyde (5.0 g, 15.6 mmol), bis(pinacolato)diboron (11.9 g, 46.9 mmol), PdCl₂(dppf)₂ (377 mg, 0.47 mmol) and KOAc (4.6 g, 46.9 mmol) in 1,4-dioxane (100 mL) was stirred at 80° C. overnight under argon. The organic solvent was removed and the residue was purified by column chromatography over silica gel (eluent: petroleum ether/EtOAc 10/1) to afford 4-(3-bromopropoxy)-2-(4,4,5,5-tetramethyl-1,3,2-dioxaborolan-2-yl)benzaldehyde (4.87 g). Yield 84%.

### 6-(3-bromopropoxy)benzo[c][1,2]oxaborol-1(3H)-ol

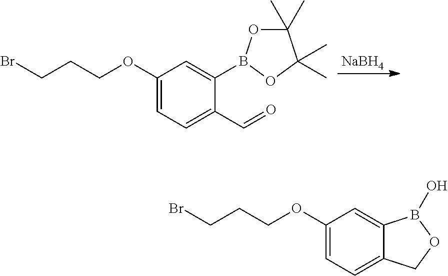

To the solution of 4-(3-bromopropoxy)-2-(4,4,5,5-tetramethyl-1,3,2-dioxaborolan-2-yl)benzaldehyde (4.87 g, 13.2 mmol) in DCM (50 ml) was added NaBH₄ (300 mg, 7.9 mmol) in MeOH (5 ml). The reaction mixture was stirred at ambient temperature for 0.5 h. The solvent was evaporated. The residue was purified by preparation HPLC to afford 6-(3-bromopropoxy)benzo[c][1,2]oxaborol-1(3H)-ol (2.2 g, 62%).

### 1-Cyclopropyl-6-fluoro-7-(4-(3-(1-hydroxy-1,3-dihydrobenzo[c][1,2]oxaborol-6-yloxy)propyl)piperazin-1-yl)-4-oxo-1,4-dihydroquinoline-3-carboxylic acid

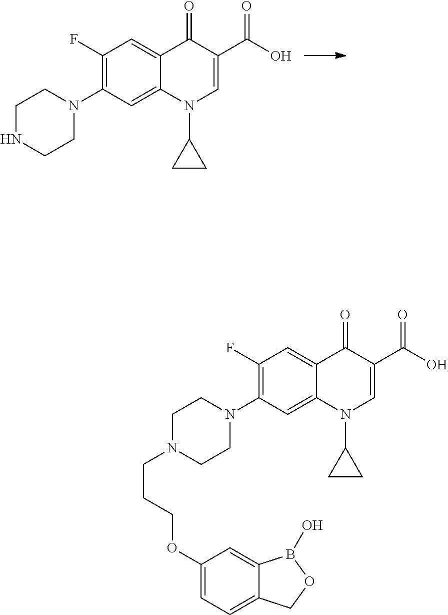

A mixture of ciprofloxacin (943 mg, 2.85 mmol) (purchased from TCI America (Portland, Oreg.), 6-(3-bromopropoxy)benzo[c][1,2]oxaborol-1(3H)-ol (1.0 g, 3.7 mmol), and triethylamine (2.4 ml, 17 mmol) in 50 ml ethanol was stirred at 95° C. overnight. The solvent was removed and the residue was purified by preparative HPLC to afford the title compound (115 mg, yield 6%) as TFA salt. HPLC purity: 98.6% at 214 nm and 100% at 254 nm; MS: m/z=522 (M+1, ESI+); ¹H NMR (DMSO-d₆, 500 MHz): δ 15.13 (s, 1H), 9.99 (s, 1H), 9.18 (s, 1H), 8.70 (s, 1H), 7.99 (d, 1H, J=13 Hz), 7.63 (d, 1H, J=7.5 Hz), 7.34 (d, 1H, J=9.0 Hz), 7.27 (d, 1H, J=2.5 Hz), 7.08 (dd, 1H, J=2.5, 8.5 Hz), 4.93 (s, 2H), 4.11 (t, 2H, J=5.5, 6.0 Hz), 3.92 (m, 2H), 3.86 (m, 1H), 3.73 (m, 2H), 3.41 (m, 2H), 3.30 (m, 4H), 2.21 (m, 2H), 1.33 (m, 2H) and 1.21 (m, 2H) ppm.

### (S)—N-((3-(3-fluoro-4-(4-(3-(1-hydroxy-1,3-dihydrobenzo[c][1,2]oxaborol-7-yloxy)propyl)piperazin-1-yl)phenyl)-2-oxooxazolidin-5-yl)methyl)acetamide hydrochloride (F6)

### 4-(2-Fluoro-4-nitro-phenyl)-piperazine-1-carboxylic acid tert-butyl ester

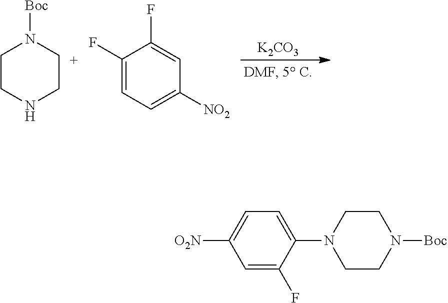

To an ice cold (5° C.) solution of piperazine-1-carboxylic acid tert-butyl ester (17.56 g, 94.28 mmol) in dry DMF (50 mL) was added K₂CO₃ (12.8 g, 94.28 mmol) and the slurry stirred for 15 minutes. 1,2-Difluoro-4-nitro-benzene (15.0 g, 94.28 mmol) was added to the slurry and the reaction mixture stirred vigorously for 2 h. The mixture was filtered and the insoluble material washed with DMF (25 mL). The combined filtrate was evaporated to dryness under reduced pressure, and the yellow solid that formed was re-crystallized from DCM to affording 30.1 g (91%) of the title compound. ¹H NMR (400 MHz, DMSO-d₆) δ (ppm): 8.04-7.99 (m, 2H), 7.17 (t, J=9.0 Hz, 1H), 3.48 (t, J=4.8 Hz, 4H), 3.25 (t, J=5.2 Hz, 4H), 1.42 (s, 9H); ¹⁹F NMR (376 MHz, DMSO-d₆) δ (ppm): −119.79 (dd, J=13.8, 9.0 Hz).

### 3-Fluoro-4 piperazin-1-yl phenylamine; hydrochloride

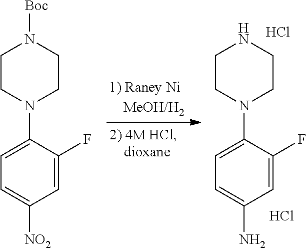

To a solution of 4-(2-fluoro-4-nitro-phenyl)-piperazine-1-carboxylic acid tert-butyl ester (29.1 g, 89.4 mmol) in MeOH (150 mL) and DCM (10 mL) mixture was added Raney Ni (5.5 g) and the Parr shaker was charged with hydrogen to 50 psi for 1 h. The initial yellow solution became clear and the content was filtered, washed with MeOH and the combined filtrate evaporated to dryness. The residue was dissolved in dry dioxane (20 mL) and a 4M HCl solution in dioxane (30 mL) was added. The solution was warmed to 45° C. in a water bath and stirred vigorously overnight producing a white precipitate. After filtering and washing with cold dioxane and diethyl ether and drying in vacuo, 23.1 g (96%) of the title compound was isolated as a white solid. ¹H NMR (400 MHz, DMSO-d₆) δ (ppm): 10.5 (br. s., 1H), 9.63 (br. s., 2H), 8.0 (br. s., 2H), 7.29 (d, J=12.6 Hz, 1H), 7.20-7.19 (m, 2H), 3.24 (d, J=4.8 Hz, 4H), 3.19 (br. s., 4H). ¹⁹F NMR (376 MHz, DMSO-d₆) δ (ppm): −120.36 (dd, J=13.6, 7.2 Hz, 1F).

### 4-(4-Benzyloxycarbonylamino-2-fluoro-phenyl)-piperazine-1-carboxylic acid benzyl ester

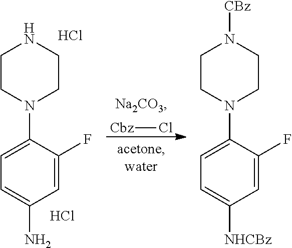

To a cold (5° C.) solution of 3-fluoro-4-piperazin-1-yl-phenylamine; dihydrochloride salt (28.0 g, 104 mmol) in acetone/water (1:1 v/v, 200 mL) was added a 10% Na₂CO₃ aqueous solution (250 mL). After 15 min, benzyl chloroformate (29.4 mL, 208 mmol) was added drop-wise to the solution and the resulting mixture stirred vigorously overnight affording a thick white precipitate. After filtration and washing with 25% acetone/water and drying, 40.0 g (85%) of the title compound was isolated as a white powder. ¹H NMR (400 MHz, CDCl₃) δ (ppm): 7.40-7.28 (m, 11H), 6.96 (d, J=7.2 Hz, 1H), 6.84 (t, J=9.2 Hz, 1H), 6.61 (br. s., 1H), 5.18 (s, 2H), 5.15 (s, 2H), 3.66 (t, J=5.0 Hz, 4H), 2.98 (br. s., 4H); ¹⁹F NMR (376 MHz, CDCl₃) δ (ppm): −122.22 (dd, J=15.4, 10.6 Hz); MS (ESI) m/z=464 (M+1, positive).

### (R)-benzyl 4-(2-fluoro-4-(5-(hydroxymethyl)-2-oxooxazolidin-3-yl)phenyl)piperazine-1-carboxylate

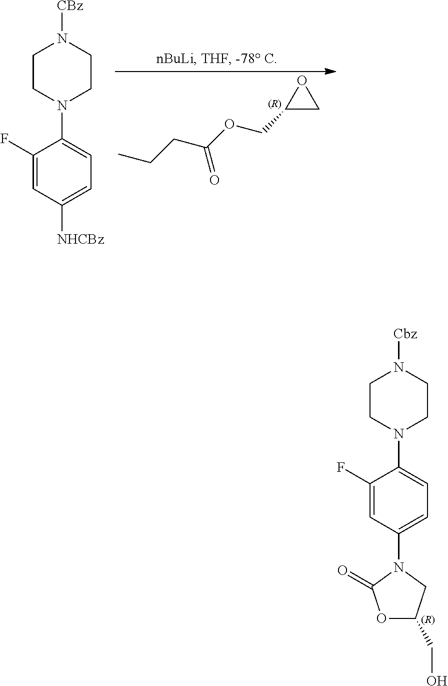

To a solution of 4-(4-benzyloxycarbonylamino-2-fluoro-phenyl)-piperazine-1-carboxylic acid benzyl ester (5.0 g, 10.78 mmol) in dry THF (50 mL) at −78° C. was added a 2.5 M solution of n-butyllithium in hexane (4.5 mL, 10.78 mmol) and the resulting solution stirred for 1 h at −78° C. (R)-Glycidyl butyrate (1.55 g, 10.78 mmol) was added to the solution at −60° C. and the solution warmed to room temperature overnight. To the reaction was added a saturated NH₄Cl solution (5 mL) and the aqueous layer extracted with EtOAc. The combined organic layer was dried over Na₂SO₄, filtered and concentrated in vacuo. The residue was triturated with 30% EtOAc/hexanes at 40° C., sonicated for 15 minutes and cooled to 5° C. The powder generated was isolated by filtration, washed with cold EtOAc/hexane (1:1, v/v) and dried to afford 4.0 g (87%) of the title compound. ¹H NMR (400 MHz, CDCl₃) δ (ppm): 7.46 (dd, J=14.0, 2.4 Hz, 1H), 7.38-7.31 (m, 5H), 7.12 (dd, J=9.0, 1.8 Hz, 1H), 6.91 (t, J=9.2 Hz, 1H), 5.16 (s, 2H), 4.77-4.71 (m, 1H), 4.02-3.92 (m, 3H), 3.77-3.69 (br. s., 1H), 3.68-3.66 (m, 4H), 3.00 (br. s., 4H), 2.25 (brs, 1H). ¹⁹F NMR (376 MHz, CDCl₃) δ (ppm): −120.66 (dd, J=14.6, 9.8 Hz, 1F); MS (ESI) m/z=430 (M+1, positive).

### (R)-benzyl 4-(2-fluoro-4-(2-oxo-5-(tosyloxymethyl)oxazolidin-3-yl)phenyl)piperazine-1-carboxylate

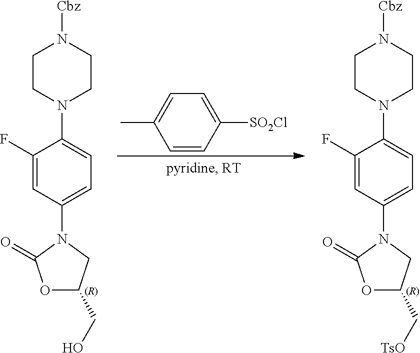

4-[2-Fluoro-4-(5-hydroxymethyl-2-oxo-oxazolidin-3-yl)-phenyl]-piperazine-1-carboxylic acid benzyl ester (2.7 g, 6.28 mmol) was dissolved in dry pyridine (50 mL) and the mixture was stirred for 15 minutes before cooling to 5° C. p-Toluenesulfonyl chloride (1.19 g, 6.28 mmol) was added and the resulting solution warmed to room temperature overnight. Pyridine was evaporated partially in vacuo and water (150 mL) was added. After sonicating for 2 h, the resulting solid was filtered, washed with cold EtOAc and dried in vacuo, affording 3.0 g (81%) of the title compound as a white solid. ¹H NMR (400 MHz, CDCl₃) δ (ppm): 7.78 (d, J=8.0 Hz, 2H), 7.40-7.33 (m, 8H), 7.06 (d, J=8.8 Hz, 1H), 6.91 (t, J=9.2 Hz, 1H), 5.16 (s, 2H), 4.83-4.79 (m, 1H), 4.25-4.20 (m, 2H), 4.06 (t, J=9.2 Hz, 1H), 3.86 (dd, J=8.8, 6.0 Hz, 1H), 3.69-3.67 (m, 4H), 3.01 (br. s., 4H), 2.46 (s, 3H); ¹⁹F NMR (376 MHz, CDCl₃) δ (ppm): −120.40 (dd, J=14.8, 9.6 Hz, 1F); MS (ESI) m/z=583 (M+1, positive).

### (R)-benzyl 4-(4-(5-(azidomethyl)-2-oxooxazolidin-3-yl)-2-fluorophenyl)piperazine-1-carboxylate

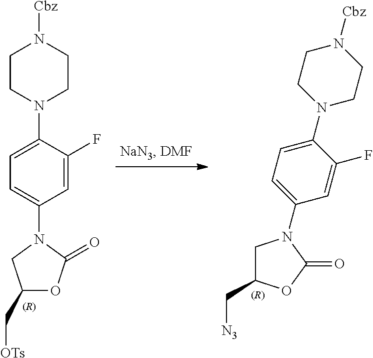

To a solution of 4-{2-fluoro-4-[2-oxo-5-(toluene-4-sulfonyloxymethyl)-oxazolidin-3-yl]-phenyl}-piperazine-1-carboxylic acid benzyl ester (3.2 g, 5.48 mmol) in dry DMF (50 mL) was added NaN₃ (2.14 g, 32.89 mmol) and the mixture was heated to 65° C. for 24 h. The solvent was evaporated partially under vacuum to afford a semi solid, which was dissolved in EtOAc and washed with water twice. The organic layer was dried over Na₂SO₄, filtered and evaporated under reduced pressure to provide 2.2 g (88%) of the title compound as a white semi-solid. ¹H NMR (400 MHz, CDCl₃) δ (ppm): 7.44 (dd, J=14.4, 2.4 Hz, 1H), 7.37-7.31 (m, 5H), 7.10 (dd, J=8.4, 1.2 Hz, 1H), 6.91 (t, J=9.0 Hz, 1H), 5.15 (s, 2H), 4.78-4.74 (m, 1H), 4.03 (t, J=9.0 Hz, 1H), 3.80 (dd, J=9.0, 6.2 Hz, 1H), 3.71-3.65 (m, 5H), 3.56 (dd, J=13.2, 4.4 Hz, 1H), 3.00 (br. s., 4H); ¹⁹F NMR (376 MHz, CDCl₃) δ (ppm): −120.51 (dd, J=15.2, 10.4 Hz, 1F); MS (ESI) m/z=454 (M+1, positive).

### (S)-benzyl 4-(4-(5-(aminomethyl)-2-oxooxazolidin-3-yl)-2-fluorophenyl)piperazine-1-carboxylate

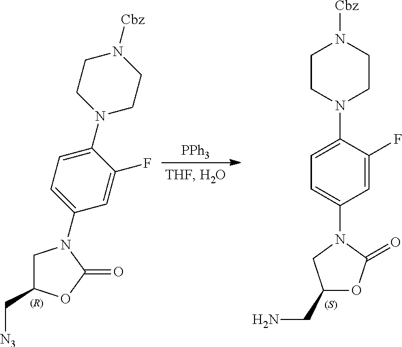

To a solution of 4-[4-(5-azidomethyl-2-oxo-oxazolidin-3-yl)-2-fluoro-phenyl]-piperazine-1-carboxylic acid benzyl ester (2.6 g, 5.72 mmol) in dry THF (50 mL) was added PPh₃ (3.0 g, 11.44 mmol). After 4 h, an additional PPh₃ (1.0 g) was added and the mixture stirred for 3 h. Water (1.5 mL) was added and the reaction mixture heated at reflux for 12 h. After cooling to r.t., the solvent was removed in vacuo and the residue (2.7 g) was directly taken to next step without further purification.

### (S)-benzyl 4-(4-(5-(acetamidomethyl)-2-oxooxazolidin-3-yl)-2-fluorophenyl)piperazine-1-carboxylate

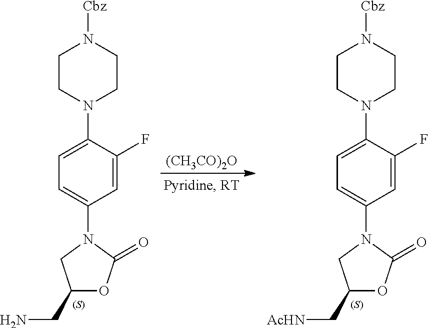

To a solution of 4-[4-(5-aminomethyl-2-oxo-oxazolidin-3-yl)-2-fluoro-phenyl]-piperazine-1-carboxylic acid benzyl ester (5.5 g, 12.83 mmol) in dry DCM (50 mL) was added pyridine (2.0 mL, 25.66 mmol) and the solution stirred for 15 minutes before cooling to 5° C. Acetic anhydride (3.0 mL, 27.2 mmol) was added drop-wise to the solution and the reaction stirred at room temperature overnight. The reaction was diluted with DCM (100 mL) and the organic layer washed successively with 1N HCl, dilute NaHCO₃ solution and brine. The organic layer was dried over Na₂SO₄, filtered and the solvent evaporated to dryness under reduced pressure. The solid obtained was treated with EtOAc (20 ml) and sonicated at 50° C. for 30 minutes. The resulting cloudy solution was cooled to 5° C. to afford a white powdery material, which was collected by filtration and dried in vacuo generating 4.53 g (75%) of the title compound. ¹H NMR (400 MHz, CDCl₃) δ (ppm): 7.43 (dd, J=14.2, 2.6 Hz, 1H), 7.37-7.29 (m, 5H), 7.06 (dd, J=9.0, 1.8 Hz, 1H), 6.90 (t, J=9.2 Hz, 1H), 6.20 (t, J=6.0 Hz, 1H), 5.16 (s, 2H), 4.77-4.73 (m, 1H), 4.00 (t, J=9.0 Hz, 1H), 3.74 (dd, J=9.0, 6.6 Hz, 2H), 3.68-3.66 (m, 4H), 3.62 (t, J=6.0 Hz, 1H), 3.00 (br. s., 4H), 2.01 (s, 3H). ¹⁹F NMR (376 MHz, CDCl₃) δ (ppm): −120.43 (dd, J=15.4, 10.2 Hz, 1F); MS (ESI) m/z=471 (M+1, positive).

### (S)—N-((3-(3-fluoro-4-(piperazin-1-yl)phenyl)-2-oxooxazolidin-5-yl)methyl)acetamide

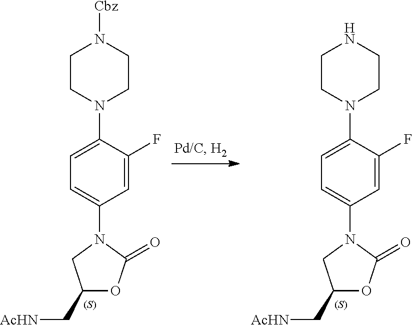

To a solution of 4-{4-[5-(acetylamino-methyl)-2-oxo-oxazolidin-3-yl]-2-fluoro-phenyl}-piperazine-1-carboxylic acid benzyl ester (3.2 g, 6.80 mmol) in MeOH (20 mL) and DCM (5 mL) was added 10% Pd/C (0.70 g) and hydrogen gas was bubbled through the solution for 30 minutes and left under a balloon of hydrogen for 12 h. The reaction was filtered, washed with MeOH, DCM and EtOAc successively and the combined filtrate evaporated to dryness under reduced pressure affording a foamy solid. This solid was treated with 20 mL of 10% MeOH/EtOAc and sonicated at 50° C. for 30 minutes. The resulting cloudy solution was cooled to 5° C. and the white precipitate formed was filtered and washed with cold EtOAc affording 1.3 g (57%) of the title compound as a white solid. ¹H NMR (400 MHz, DMSO-d₆) δ (ppm): 8.61 (br. s., 1H), 8.26 (t, J=6.0 Hz, 1H), 7.50 (dd, J=14.8, 2.8 Hz, 1H), 7.19 (dd, J=9.0, 2.2 Hz, 1H), 7.11 (t, J=9.2 Hz, 1H), 4.72-4.67 (m, 1H), 4.08 (t, J=8.8 Hz, 1H), 3.71 (dd, J=9.2, 6.4 Hz, 1H), 3.39 (t, J=5.6 Hz, 2H), 3.14 (br. s., 8H), 1.82 (s, 3H); ¹⁹F NMR (376 MHz, DMSO-d₆) δ (ppm): −121.57 (dd, J=15.8, 11.0 Hz, 1F); MS (ESI) m/z=337 (M+1, positive).

### (S)—N-((3-(3-fluoro-4-(4-(3-(1-hydroxy-1,3-dihydrobenzo[c][1,2]oxaborol-7-yloxy)propyl)piperazin-1-yl)phenyl)-2-oxooxazolidin-5-yl)methyl)acetamide hydrochloride

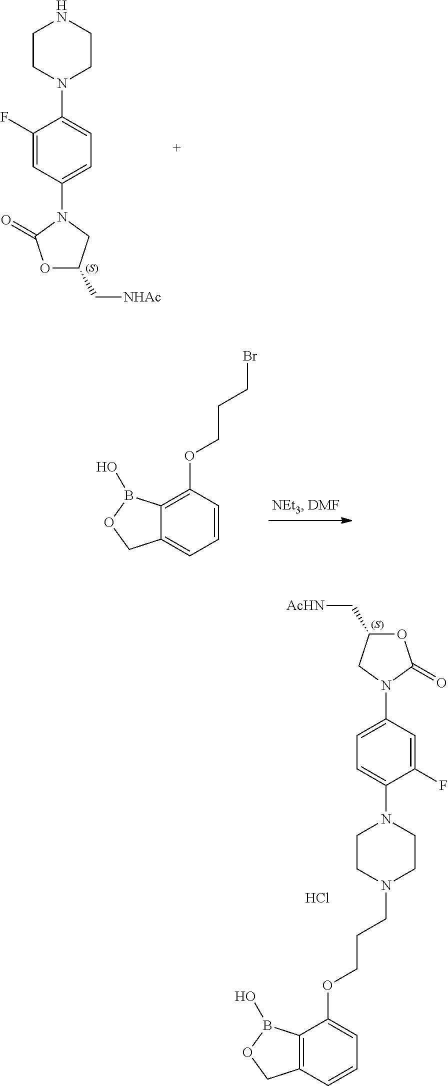

To a solution of 7-(3-bromo-propoxy)-3H-benzo[c][1,2]oxaborol-1-ol (0.33 g, 1.21 mmol) in dry DMF (8 mL) was added a solution of N-[3-(3-fluoro-4-piperazin-1-yl-phenyl)-2-oxo-oxazolidin-5-ylmethyl]-acetamide (0.41 g, 1.21 mmol) and NEt₃ (0.34 mL, 2.42 mmol) in DMF (4 mL). The resulting solution heated at 40° C. for 1 h and stirred at room temperature for 2 h. The volatiles were removed in vacuo and purification was accomplished by preparative HPLC using water (0.1% acetic acid) and MeOH gradient. The lyophilized material obtained was dissolved in a 2N HCl solution (20 mL) and re-lyophilized generating a white powder which was again treated with H₂O (5 mL), sonicated for 15 minutes and filtered producing 0.097 g (15%) of the title compound as a white powder. ¹H NMR (400 MHz, DMSO-d₆) δ (ppm): 10.54 (s, 1H), 8.78 (s, 1H), 8.27 (t, J=5.8 Hz, 1H), 7.52 (dd, J=14.6, 2.6 Hz, 1H), 7.44 (t, J=7.8 Hz, 1H), 7.22 (dd, J=8.8, 2.4 Hz, 1H), 7.15 (t, J=9.2 Hz, 1H), 6.98 (d, J=7.6 Hz, 1H), 6.86 (d, J=8.0 Hz, 1H), 4.94 (s, 2H), 4.74-4.68 (m, 1H), 4.16 (t, J=5.8 Hz, 2H), 4.09 (t, J=9.0 Hz, 1H), 3.71 (dd, J=9.2, 6.4 Hz, 1H), 3.65-3.62 (br. s., 2H), 3.46-3.35 (m, 5H), 3.27-3.13 (m, 4H), 2.18-2.30 (m, 2H), 1.83 (s, 3H); ¹⁹F NMR (376 MHz, DMSO-d₆) δ (ppm): −121.50 (dd, J=15.2, 10.4 Hz, 1F); MS (ESI) m/z=527 (M+1, positive); HPLC purity: 98.54% (MaxPlot 200-400 nm), 98.51% (220 nm).

### 7-(4-(3-(3-(Aminomethyl)-1-hydroxy-1,3-dihydrobenzo[c][1,2]oxaborol-7-yloxy)propyl)piperazin-1-yl)-1-cyclopropyl-6-fluoro-4-oxo-1,4-dihydroquinoline-3-carboxylic acid (F7)

### Step 1: 2-Bromo-3-(3-bromopropoxy)benzaldehyde

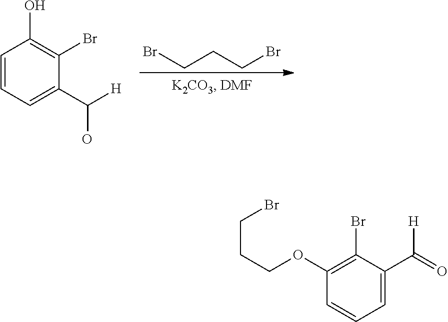

A mixture of 2-bromo-3-hydroxybenzaldehyde (2 g, 10 mmol), 1,3-dibromopropane (1.2 mL, 10 mmol), Cs₂CO₃ (5 g, 30 mmol), and DMF (10 mL) was stirred at r.t. overnight. The mixture was diluted with EtOAc, and washed with H₂O, brine, and dried over Na₂SO₄. The crude product was purified with flash chromatography (5%-20% EtOAc/hexane) to give the title compound as white solid (1.7 g, 53%). ¹H NMR (400 MHz, CDCl₃) δ 10.43 (s, 1H), 7.54 (dd, J=1.7, 8.0 Hz, 1H), 7.37 (t, J=8.0 Hz, 1H), 7.15 (dd, J=8.0, 1.7 Hz, 1H), 4.22 (t, J=5.6 Hz, 2H), 3.71 (t, J=6.3 Hz, 2H), 2.41 (m, 2H).

### Step 2: 3-(3-Bromopropoxy)-2-(4,4,5,5-tetramethyl-1,3,2-dioxaborolan-2-yl)benzaldehyde

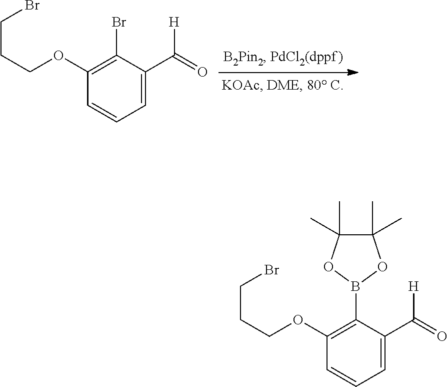

A mixture of 2-bromo-3-(3-bromopropoxy)benzaldehyde (0.8 g, 2.48 mmol), B₂Pin₂ (0.82 g, 3.22 mmol), KOAc (0.75 g, 7.45 mmol), Pd(dppf)Cl₂ (80 mg, 0.074 mmol) in DME (15 mL) under nitrogen was refluxed overnight, filtered and washed with EtOAc. After concentration, the reaction was purification by flash chromatography (10%-30% EtOAc/hexane) to give the title compound (0.2 g 22%). MS (ESI) m/z=345.1 (Boronic acid+OAc, negative). ¹H NMR (400 MHz, CDCl₃) δ 9.43 (s, 1H), 7.48 (t, J=8.0 Hz, 1H), 7.41 (d, J=7.8 Hz, 1H), 7.09 (d, J=8.0 Hz, 1H), 4.15 (t, J=5.7 Hz, 2H), 3.61 (t, J=6.6 Hz, 2H), 2.31 (m, 2H), 1.45 (s, 9H).

### Step 3: 7-(3-Bromopropoxy)-3-(nitromethyl)benzo[c][1,2]oxaborol-1(3H)-ol

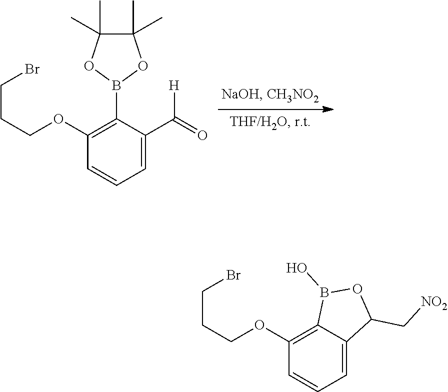

To a mixture of NaOH (30.8 mL, 1M in water) and nitromethane (2.6 mL, 46.3 mmol) in THF (48 mL) and water (16 mL) was added 3-(3-bromopropoxy)-2-(4,4,5,5-tetramethyl-1,3,2-dioxaborolan-2-yl)benzaldehyde (5.7 g, 15.5 mmol), the reaction was stirred at r.t. for 6 h then added 1N HCl (46 mL) and stirred overnight. The reaction was worked up with EtOAc, H₂O, brine and dried over Na₂SO₄. Flash column chromatography (20%-50% EtOAc/hexane) gave the title compound (4.0 g, 78%) as yellow solid. ¹H NMR (400 MHz, CDCl₃) δ 7.49 (t, J=7.8 Hz, 1H), 6.92 (d, J=7.6 Hz, 1H), 6.87 (d, J=8.0 Hz, 1H), 5.85 (dd, J=9.0, 3.5 Hz, 1H), 5.11 (s, 1H), 4.74 (dd, J=13.3, 3.5 Hz, 1H), 4.45 (dd, J=13.1, 9.0 Hz, 1H), 4.24 (t, J=5.9 Hz, 2H), 3.61 (t, J=6.2 Hz, 2H), 2.38 (m, 2H).

### Step 4: [7-(3-Bromo-propoxy)-1-hydroxy-1,3-dihydro-benzok[c][1,2]oxaborol-3-ylmethyl]-carbamic acid tert-butyl ester

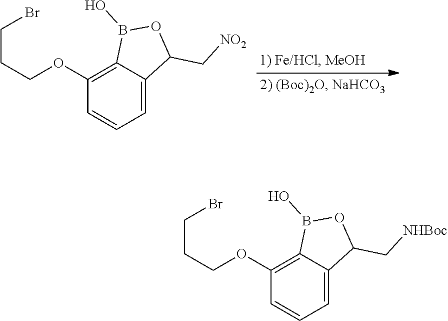

To a suspension of 7-(3-bromopropoxy)-3-(nitromethyl)benzo[c][1,2]oxaborol-1(3H)-ol (500 mg, 1.5 mmol) and iron powder (678 mg, 12.1 mmol) in MeOH (20 mL) was added HCl (20 mL, ˜1.5 M in MeOH), and the resulting mixture was stirred at r.t. overnight. The reaction mixture was filtered and washed with MeOH. The filtrate was concentrated and dissolved in EtOAc. The resulting solution was washed with sat. NaHCO₃, brine and dried over Na₂SO₄. After concentration, the residue was dissolved in THF (10 mL) and water (5 mL). (Boc)₂O (362 mg, 1.66 mmol) and NaHCO₃ (380 mg, 4.53 mmol) were added. The resulting mixture was stirred at r.t. for 5 hours, and then extracted with dichloromethane (3×30 mL). The organic solution was concentrated and purified by flash column chromatography to give the title compound (400 mg) as yellow solid. LC-MS (ESI) m/z=341.1, 343.1 (M-Boc+1+MeCN, positive).

### Step 5: 7-(4-(3-(3-((tert-butoxycarbonylamino)methyl)-1-hydroxy-1,3-dihydrobenzo[c][1,2]oxaborol-7-yloxy)propyl)piperazin-1-yl)-1-cyclopropyl-6-fluoro-4-oxo-1,4-dihydroquinoline-3-carboxylic acid

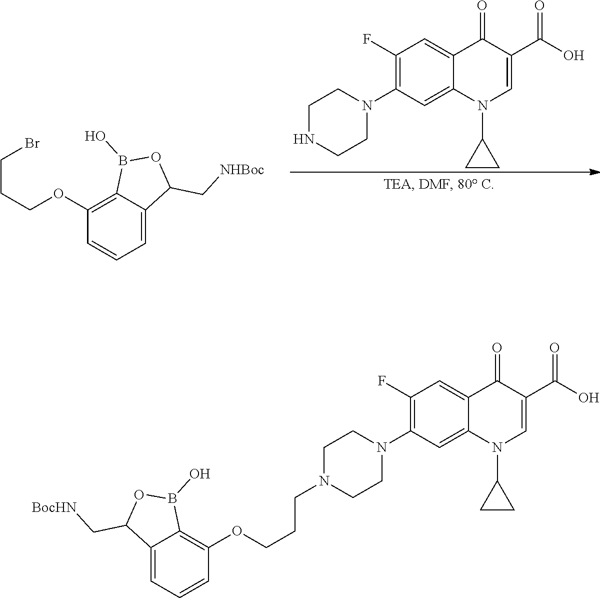

A mixture of [7-(3-bromo-propoxy)-1-hydroxy-1,3-dihydro-benzo[c][1,2]oxaborol-3-ylmethyl]-carbamic acid tert-butyl ester (360 mg, 0.9 mmol), ciprofloxacin hydrochloride (366 mg, 0.99 mmol), and Et₃N (0.474 mL, 3.4 mmol) in DMF (10 mL) was heated at 80° C. for 3 hours. After the reaction mixture was cooled down, it was partitioned in EtOAc/H₂O. The organic layer was washed with water, brine and then dried over Na₂SO₄. The resulting solution was concentrated and purified by flash column chromatography (DCM:MeOH, 10:1) to give the title compound (320 mg) as yellow solid. LC-MS (ESI) m/z=651.6 (M+1, positive).

### Step 6: 7-(4-(3-(3-(aminomethyl)-1-hydroxy-1,3-dihydrobenzo[c][1,2]oxaborol-7-yloxy)propyl)piperazin-1-yl)-1-cyclopropyl-6-fluoro-4-oxo-1,4-dihydroquinoline-3-carboxylic acid

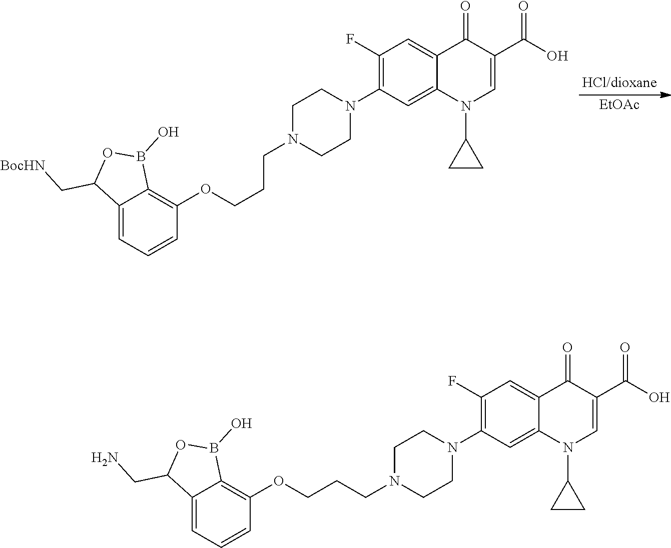

To a mixture of 7-(4-(3-(3-((tert-butoxycarbonylamino)methyl)-1-hydroxy-1,3-dihydrobenzo[c][1,2]oxaborol-7-yloxy)propyl)piperazin-1-yl)-1-cyclopropyl-6-fluoro-4-oxo-1,4-dihydroquinoline-3-carboxylic acid (56 mg, 0.086 mmol) in ethyl acetate (5 mL) was added HCl (4N in dioxane, 1 mL). The mixture was stirred at room temperature for 5 h and concentrated. Purification of the residue with prep-HPLC gave the title compound (14 mg). LC-MS (ESI) m/z 551.3 (M+1, positive). ¹H NMR (300 MHz, DMSO-d₆) δ 8.64 (s, 1H), 8.25 (s, 2H), 7.87 (d, J=13.2 Hz, 1H), 7.53 (d, J=7.8 Hz, 1H), 7.43 (t, J=8.1 Hz, 1H), 6.99 (d, J=7.5 Hz, 1H), 6.88 (d, J=8.4 Hz, 1H), 5.19 (br d, 1H), 4.10-3.95 (m, 3H), 3.77 (br s, 2H), 3.32 (br s, 4H), 3.19 (br s, 1H), 2.60 (br s, 4H), 1.93 (br t, 2H), 1.29 (br d, 2H), 1.13 (br s, 2H).

### Example 2

### LeuRS IC50 Testing

Experiments were performed in 96-well microtiter plates, using 80 μL reaction mixtures containing 50 mM HEPES-KOH (pH 8.0), 30 mM MgCl₂ and 30 mM KCl, 13 μM [¹⁴C]leucine (306 mCi/mmol, Perkin-Elmer), 15 uM total E. coli tRNA (Roche, Switzerland), 0.02% (w/v) BSA, 1 mM DTT, 0.2 pM LeuRs and 4 mM ATP at 30° C. Reactions were started by the addition of 4 mM ATP. After 7 minutes, reactions were quenched and tRNA was precipitated by the addition of 50 μL of 10% (w/v) TCA and transferred to 96-well nitrocellulose membrane filter plates (Millipore Multiscreen HTS, MSHAN4B50). Each well was then washed three times with 100 μL of 5% TCA. Filter plates were then dried under a heat lamp and the precipitated [¹⁴C]leucine tRNA^{Leu} were quantified by liquid scintillation counting using a Wallac MicroBeta Trilux model 1450 liquid scintillation counter (PerkinElmer, Waltham Mass.).

To determine the inhibitor concentration which reduces enzyme activity by 50% (IC₅₀), increasing concentrations of inhibitor were incubated with LeuRS enzyme, tRNA and leucine for 20 minutes. Reactions were initiated by the addition of 4 mM ATP and stopped after 7 minutes then precipitated and counted to quantify radioactivity.

### Example 3

### Antibacterial MIC Testing

All MIC testing of bacteria followed the Clinical and Laboratory Standards Institute (CLSI) guidelines for antimicrobial testing of aerobic bacteria (Methods for Dilution Antimicrobial Susceptibility Tests for Bacteria That Grow Aerobically; Approved Standard—Seventh Edition) (M07-A7) and anaerobic bacteria (Methods for Antimicrobial Susceptibility Testing of Anaerobic Bacteria; Approved Standard—Seventh Edition) (M11-A7).

### Example 4

### Assay for Determining that a Compound Inhibits the Editing Domain of tRNA Synthetase in a Bacteria

This example sets forth a representative assay for determining whether a particular compound inhibits the editing domain of an ARS in a bacterium.

The [³H]-isoleucine mischarged tRNAleu was synthesized by incubating 1 μM of Saccharomyces cerevisiae editing defective Cdc60p (C326F) in 500 μl of 50 mM Tris-HCl (pH 8.0), 60 mM MgCl₂, 4 mM ATP, 1 mM DTT, 0.02% (w/v) BSA, 4 mg/mL crude E. coli tRNA tRNA (Roche), 0.1 mM isoleucine and 5 mCi L-[4,5-3H]isoleucine (100 Ci/mmole, GE Healthcare) and 20% (v/v) DMSO for 1 hour at 30° C. The reaction was stopped by adding 10 μL of 10% (v/v) acetic acid followed by two acidic phenol (Sigma) extractions. The mischarged tRNA in the top aqueous phase was removed and precipitated by adding two volumes of 96% (v/v) ethanol and incubating at −20° C. for 30 minutes. The precipitate was pelleted by centrifugation at 13,200×g for 30 minutes and the mischarged tRNA pellet was washed twice with 70% (v/v) ethanol and then resuspended in 50 mM potassium phosphate buffer pH 5.2.

The reaction was terminated after 2 hours incubation at 30° C. by the addition of acetic acid to 0.17% (v/v). The isoleucylated crude tRNA^{Leu} was purified by extracting twice with acidic phenol-chloroform extractions (pH 4.3), followed by ethanol precipitation. The tRNA pellet was washed twice with 70% ethanol, dried and then resuspended in 50 mM potassium phosphate (pH 5.0) and stored at −20° C. An aliquot was precipitated with 10% (w/v) TCA to quantify ile-tRNA^{Leu}.

Post-transfer editing hydrolysis assays were carried out at 30° C. in 50 mM Hepes (pH 8), 10 mM MgCl₂, 30 mM KCl, with ³H-isoleucine-tRNA crude (˜0.3 μCi/mL). Each reaction was initiated by addition of the 150 nM enzyme. At each time point three 20 μL aliquots of the reaction mixture was added to 200 μL of 10% (w/v) TCA in a Millipore filter plate and precipitated for 20 minutes at 4° C. The precipitate was filtered and washed three times with 200 μL of 5% (w/v) TCA, then dried and 20 μL Supermix scintillation cocktail was added. The Millipore filter plates were counted in the MicroBeta Trilux. The IC₅₀ was determined by the amount of inhibitor that inhibited 50% activity, 100% post-transfer editing was calculated by taking the activity of the no enzyme control from the wild-type enzyme activity.

It is to be understood that the invention covers all combinations of aspects with all other suitable aspects and/or exemplary embodiments described herein. It is to be understood that the invention also covers all combinations of exemplary embodiments with all other suitable aspects and/or exemplary embodiments described herein.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having a structure which is: 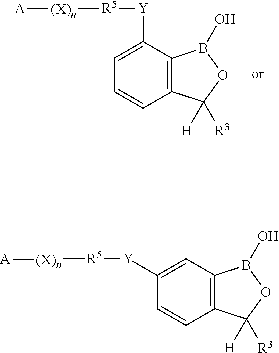
wherein
Y is O or S;
R⁵ is selected from the group consisting of substituted or unsubstituted alkylene and substituted or unsubstituted heteroalkylene;
R³ is selected from the group consisting of H, cyano, substituted or unsubstituted nitroalkyl and substituted or unsubstituted aminoalkyl;
A is an antibiotic or its pharmacophore;
n is 0 or 1 or 2 or 3 or 4 or 5;
each X are each the same or different and each are selected from the group consisting of: —(C₁ or C₂ or C₃ or C₄ or C₅ or C₆)alkylene, —(C₁ or C₂ or C₃ or C₄ or C₅ or C₆)alkenylene, —(C₁ or C₂ or C₃ or C₄ or C₅ or C₆)alkynylene, —(C₃ or C₄ or C₅ or C₆ or C₇ or C₈)cycloalkylene, —O—, —C(H)═—, —C(═O)—, —C(═N—O—R¹³)—, wherein R¹³ represents hydrogen, (C₁ or C₂ or C₃ or C₄ or C₅ or C₆)alkyl, substituted (C₁ or C₂ or C₃ or C₄ or C₅ or C₆)alkyl, —S(O)ₘ—, wherein m is 0, 1, or 2, —N(R¹⁴)—, wherein R¹⁴ represents hydrogen, (C₁ or C₂ or C₃ or C₄ or C₅ or C₆)alkyl, substituted (C₁ or C₂ or C₃ or C₄ or C₅ or C₆)alkyl; arylene, heteroarylene, and bivalent heterocyclic structure containing 1 or 2 or 3 heteroatoms, wherein, the carbon or nitrogen atoms of X can be optionally substituted by 1 or 2 or 3 substituents, each of which is selected from the group consisting of (C₁ or C₂ or C₃ or C₄ or C₅ or C₆)alkyl, substituted (C₁ or C₂ or C₃ or C₄ or C₅ or C₆)alkyl, heterocycloalkyl, amino, (C₁ or C₂ or C₃ or C₄ or C₅ or C₆)alkylamino, di(C₁ or C₂ or C₃ or C₄ or C₅ or C₆)alkylamino, hydroxyl, and (C₁ or C₂ or C₃ or C₄ or C₅ or C₆)alkoxy;
or a salt thereof.

2. The compound of claim 1, wherein A comprises a moiety which is a quinolone or an oxazolidinone.

3. The compound of claim 2, wherein A comprises an oxazolidinone, and the oxazolidinone is selected from the group consisting of linezolid, torezolid, posizolid, ranbezolid, radezolid and eperezolid.

4. The compound of claim 1, wherein said compound has a structure which is: 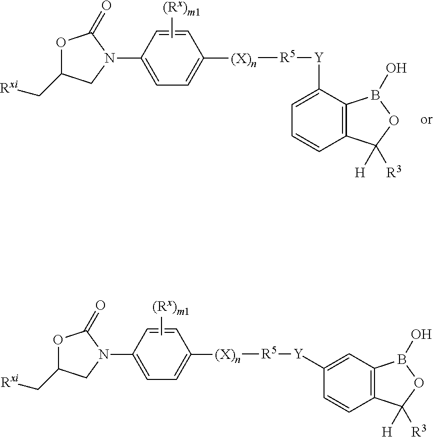
wherein
R^{x} is halogen;
R^{xi} is selected from the group consisting of CH₃CONH—, OH and substituted or unsubstituted triazole
m1 is 0 or 1 or 2.

5. The compound of claim 4, wherein said compound has a structure which is:

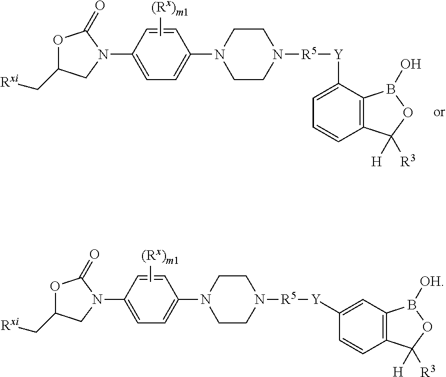

6. The compound of claim 2, wherein A is a quinolone, and the quinolone is fluoroquinolone or non-fluoroquinolone.

7. The compound of claim 2, wherein A is a quinolone, which is selected from the group consisting of nalidixic acid, oxolinic acid, piromidic acid, pipemidic acid, cinoxacin and flumequine.

8. The compound of claim 2, wherein A is a quinolone, which is selected from the group consisting of ciprofloxacin, enoxacin, fleroxacin, lomefloxacin, nadifloxacin, norfloxacin, ofloxacin, pefloxacin and rufloxacin.

9. The compound of claim 2, wherein A is a quinolone, which is selected from the group consisting of balofloxacin, gatifloxacin, grepafloxacin, levofloxacin, moxifloxacin, pazufloxacin, sparfloxacin, temafloxacin and tosufloxacin.

10. The compound of claim 2, wherein A is a quinolone, which is selected from the group consisting of clinafloxacin, besifloxacin, gemifloxacin, sitafloxacin, alatrofloxacin, trovafloxacin and prulifloxacin.

11. The compound of claim 2, wherein A is a quinolone, which is garenoxacin or delafloxacin.

12. The compound of claim 2, wherein A is a quinolone, which is selected from the group consisting of danofloxacin, difloxacin, enrofloxacin, ibafloxacin, marbofloxacin, rosoxacin, orbifloxacin and sarafloxacin.

13. The compound of claim 1, wherein said compound has a structure which is selected from the group consisting of: 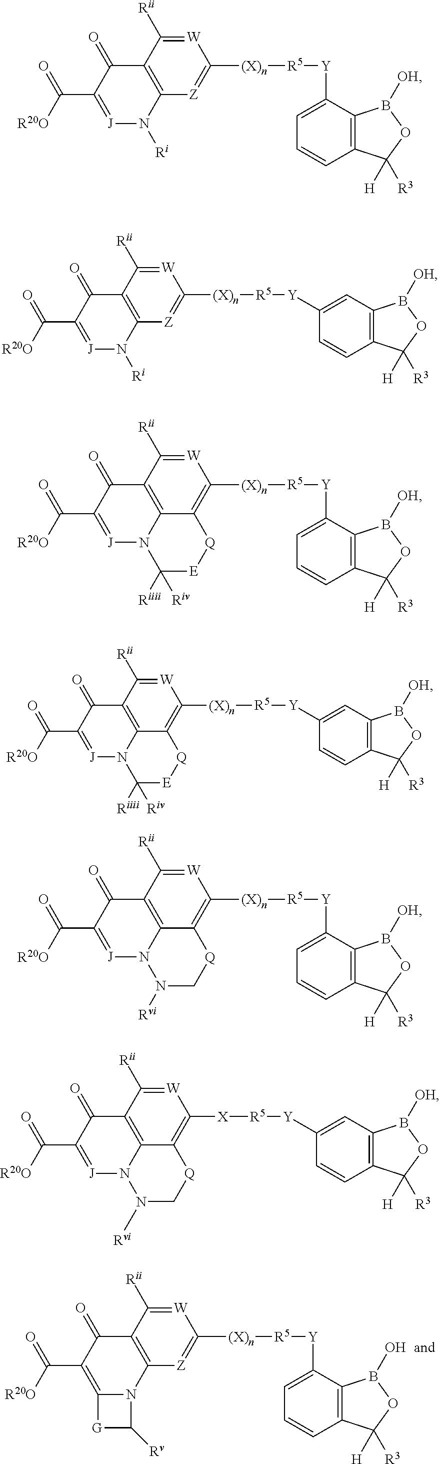 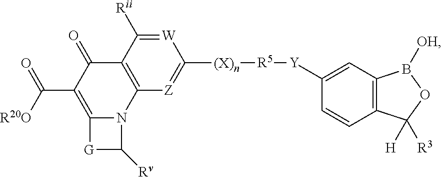
wherein
R²⁰ is selected from the group consisting of H, unsubstituted alkyl, benzyl or a negative charge; Rⁱ is (C₁ or C₂ or C₃ or C₄ or C₅ or C₆)alkyl, halosubstituted (C₁ or C₂ or C₃ or C₄ or C₅ or C₆)alkyl, (C₃ or C₄ or C₅ or C₆)cycloalkyl, halosubstituted (C₃ or C₄ or C₅ or C₆)cycloalkyl, aryl, substituted aryl, heteroaryl, and halo-substituted heteroaryl;
Rⁱⁱ is selected from the group consisting of hydrogen, halogen, amino and unsubstituted alkyl;
Rⁱⁱⁱ and R^{iv} are each independently hydrogen or (C₁ or C₂ or C₃ or C₄ or C₅ or C₆)alkyl;
W is selected from the group consisting of CH, CF, and N;
Z is selected from the group consisting of N, CN, CH, C—F, C—Cl, and CR^{vii}, wherein
R^{vii} is selected from the group consisting of unsubstituted alkyl, halosubstituted alkyl, unsubstituted alkoxy and halosubstituted alkoxy;
Q is selected from the group consisting of CH₂, O and S;
E is selected from the group consisting of CH₂, O and S;
G is O or S;
R^{v} is unsubstituted alkyl;
R^{vi} is unsubstituted alkyl; and
J is —CH or NH.

14. The compound of claim 1, wherein R³ is selected from the group consisting of H, —CH₂NH₂ and —CH₂NO₂.

15. The compound of claim 14, wherein R³ is —CH₂NH₂.

16. The compound of claim 1, having a structure which is selected from the group consisting of:

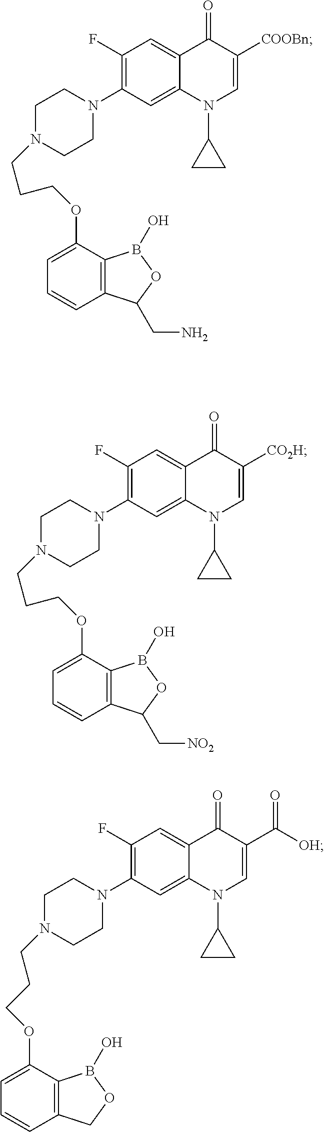

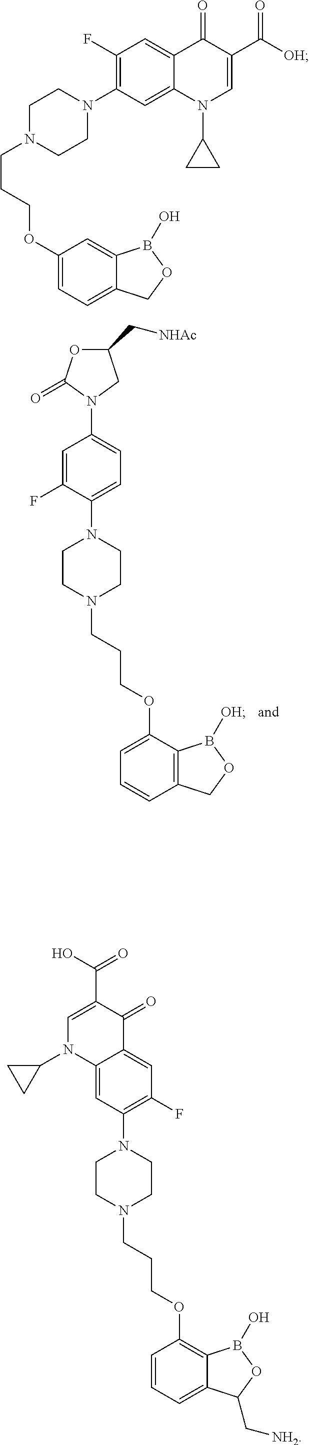

17. The compound of claim 1, having a structure which is 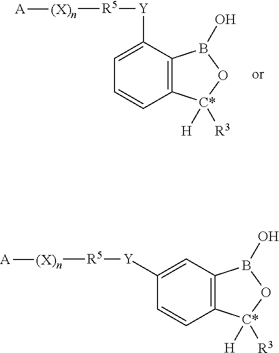
wherein C* is a carbon atom
with the proviso that when R³ is not H, C* is a stereocenter which has a configuration which is (R) or (S).

18. The compound of claim 17, with the proviso that when R³ is not H, C* is a stereocenter which has a configuration which is (S).

19. A composition comprising:
a) a first stereoisomer of the compound of claim 17;
b) at least one additional stereoisomer of the first stereoisomer;
wherein the first stereoisomer is present in an enantiomeric excess of at least 80% relative to said at least one additional stereoisomer.

20. The composition of claim 19, wherein said enantiomeric excess is at least 92%.

21. The composition of claim 19, wherein the C* stereocenter of the first stereoisomer is in a (S) configuration.

22. The composition of claim 19, wherein R³ is —CH₂NH₂.

23. A composition comprising the compound of claim 17, wherein R³ is not H and the C* stereocenter is in a (S) configuration, and said composition is substantially free of a compound wherein the C* stereocenter is in a (R) configuration.

24. A composition comprising the compound of claim 17, wherein the composition is substantially free of the enantiomer of the compound.

25. A combination comprising the compound of claim 1, or a pharmaceutically acceptable salt thereof, together with at least one other therapeutically active agent.

26. A pharmaceutical formulation comprising:
a) the compound of claim 1, or a pharmaceutically acceptable salt thereof; and
b) a pharmaceutically acceptable excipient.

27. The pharmaceutical formulation of claim 26 wherein the pharmaceutical formulation is a unit dosage form.

28. A method of inhibiting an enzyme, comprising: contacting the enzyme with the compound of claim 1, thereby inhibiting the enzyme.

29. The method of claim 28, wherein the enzyme is a t-RNA synthetase which comprises an editing domain.

30. The method of claim 28, wherein the enzyme is a leucyl t-RNA synthetase.

31. A method of killing and/or preventing the growth of a microorganism, comprising: contacting the microorganism with an effective amount of the compound of claim 1, thereby killing and/or preventing the growth of the microorganism.

32. The method of claim 31, wherein the microorganism is a bacterium.

33. A method of treating and/or preventing a disease in an animal, comprising: administering to the animal a therapeutically effective amount of the compound of claim 1, or a pharmaceutically-acceptable salt thereof, thereby treating and/or preventing the disease.

34. The method of claim 33, wherein the disease is caused by, or is mediated by, or involves a bacteria.

35. The method of claim 33, wherein the disease is caused by, or is mediated by, or involves a drug resistant bacterium.

36. The method of claim 33, wherein the disease is pneumonia.

37. The method of claim 33, wherein the animal is a human.

38. The compound of claim 1, wherein the salt is a pharmaceutically acceptable salt.
